SEQUENCE LISTING (427 sequences)

SEQ ID NO: 1 - protein, 596 aa, Arabidopsis thaliana
MFALSKVLRRTQRLRLGACSAVFSKDIQLGGERSFDSNSIASTKREAVPRFYEISSLSNRALSSSAGTKSDQEEDDLEDGFSELEGSKSGQGSTSSDEDEGKLSADEEEEEELDLIETDVSRKTVEKKQSELFKTIVSAPGLSIGSALDKWVEEGNEITRVEIAKAMLQLRRRRMYGRALQMSEWLEANKKIEMTERDYASRLDLTVKIRGLEKGEACMQKIPKSFKGEVLYRTLLANCVAAGNVKKSELVFNKMKDLGFPLSGFTCDQMLLLHKRIDRKKIADVLLLMEKENIKPSLLTYKILIDVKGATNDISGMEQILETMKDEGVELDFQTQALTARHYSGAGLKDKAEKVLKEMEGESLEANRRAFKDLLSIYASLGREDEVKRIWKICESKPYFEESLAAIQAFGKLNKVQEAEAIFEKIVKMDRRASSSTYSVLLRVYVDHKMLSKGKDLVKRMAESGCRIEATTWDALIKLYVEAGEVEKADSLLDKASKQSHTKLMMNSFMYIMDEYSKRGDVHNTEKIFLKMREAGYTSRLRQFQALMQAYINAKSPAYGMRDRLKADNIFPNKSMAAQLAQGDPFKKTAISDILD

SEQ ID NO: 2 - protein, 918 aa, Arabidopsis thaliana
MASTPPHWVTTTNNHRPWLPQRPRPGRSVTSAPPSSSASVSSAHLSQTTPNFSPLQTPKSDFSGRQSTRFVSPATNNHRQTRQNPNYNHRPYGASSSPRGSAPPPSSVATVAPAQLSQPPNFSPLQTPKSDLSSDFSGRRSTRFVSKMHFGRQKTTMATRHSSAAEDALQNAIDFSGDDEMFHSLMLSFESKLCGSDDCTYIIRELGNRNECDKAVGFYEFAVKRERRKNEQGKLASAMISTLGRYGKVTIAKRIFETAFAGGYGNTVYAFSALISAYGRSGLHEEAISVFNSMKEYGLRPNLVTYNAVIDACGKGGMEFKQVAKFFDEMQRNGVQPDRITFNSLLAVCSRGGLWEAARNLFDEMTNRRIEQDVFSYNTLLDAICKGGQMDLAFEILAQMPVKRIMPNVVSYSTVIDGFAKAGRFDEALNLFGEMRYLGIALDRVSYNTLLSIYTKVGRSEEALDILREMASVGIKKDVVTYNALLGGYGKQGKYDEVKKVFTEMKREHVLPNLLTYSTLIDGYSKGGLYKEAMEIFREFKSAGLRADVVLYSALIDALCKNGLVGSAVSLIDEMTKEGISPNVVTYNSIIDAFGRSATMDRSADYSNGGSLPFSSSALSALTETEGNRVIQLFGQLTTESNNRTTKDCEEGMQELSCILEVFRKMHQLEIKPNVVTFSAILNACSRCNSFEDASMLLEELRLFDNKVYGVVHGLLMGQRENVWLQAQSLFDKVNEMDGSTASAFYNALTDMLWHFGQKRGAELVALEGRSRQVWENVWSDSCLDLHLMSSGAARAMVHAWLLNIRSIVYEGHELPKVLSILTGWGKHSKVVGDGALRRAVEVLLRGMDAPFHLSKCNMGRFTSSGSVVATWLRESATLKLLILHDHITTATATTTTMKSTDQQQRKQTSFALQPLLL

SEQ ID NO: 3 - protein, 862 aa, Arabidopsis thaliana
MNLAIPNPNSHHLSFLIQNSSFIGNRRFADGNRLRFLSGGNRKPCSFSGKIKAKTKDLVLGNPSVSVEKGKYSYDVESLINKLSSLPPRGSIARCLDIFKNKLSLNDFALVFKEFAGRGDWQRSLRLFKYMQRQIWCKPNEHIYTIMISLLGREGLLDKCLEVFDEMPSQGVSRSVFSYTALINAYGRNGRYETSLELLDRMKNEKISPSILTYNTVINACARGGLDWEGLLGLFAEMRHEGIQPDIVTYNTLLSACAIRGLGDEAEMVFRTMNDGGIVPDLTTYSHLVETFGKLRRLEKVCDLLGEMASGGSLPDITSYNVLLEAYAKSGSIKEAMGVFHQMQAAGCTPNANTYSVLLNLFGQSGRYDDVRQLFLEMKSSNTDPDAATYNILIEVFGEGGYFKEVVTLFHDMVEENIEPDMETYEGIIFACGKGGLHEDARKILQYMTANDIVPSSKAYTGVIEAFGQAALYEEALVAFNTMHEVGSNPSIETFHSLLYSFARGGLVKESEAILSRLVDSGIPRNRDTFNAQIEAYKQGGKFEEAVKTYVDMEKSRCDPDERTLEAVLSVYSFARLVDECREQFEEMKASDILPSIMCYCMMLAVYGKTERWDDVNELLEEMLSNRVSNIHQVIGQMIKGDYDDDSNWQIVEYVLDKLNSEGCGLGIRFYNALLDALWWLGQKERAARVLNEATKRGLFPELFRKNKLVWSVDVHRMSEGGMYTALSVWLNDINDMLLKGDLPQLAVVVSVRGQLEKSSAARESPIAKAAFSFLQDHVSSSFSFTGWNGGRIMCQRSQLKQLLSTKEPTSEESENKNLVALANSPIFAAGTRASTSSDTNHSGNPTQRRTRTKKELAGSTA

SEQ ID NO: 4 - protein, 798 aa, Arabidopsis thaliana
MDASVVRFSQSPARVPPEFEPDMEKIKRRLLKYGVDPTPKILNNLRKKEIQKHNRRTKRETESEAEVYTEAQKQSMEEEARFQTLRREYKQFTRSISGKRGGDVGLMVGNPWEGIERVKLKELVSGVRREEVSAGELKKENLKELKKILEKDLRWVLDDDVDVEEFDLDKEFDPAKRWRNEGEAVRVLVDRLSGREINEKHWKFVRMMNQSGLQFTEDQMLKIVDRLGRKQSWKQASAVVHWVYSDKKRKHLRSRFVYTKLLSVLGFARRPQEALQIFNQMLGDRQLYPDMAAYHCIAVTLGQAGLLKELLKVIERMRQKPTKLTKNLRQKNWDPVLEPDLVVYNAILNACVPTLQWKAVSWVFVELRKNGLRPNGATYGLAMEVMLESGKFDRVHDFFRKMKSSGEAPKAITYKVLVRALWREGKIEEAVEAVRDMEQKGVIGTGSVYYELACCLCNNGRWCDAMLEVGRMKRLENCRPLEITFTGLIAASLNGGHVDDCMAIFQYMKDKCDPNIGTANMMLKVYGRNDMFSEAKELFEEIVSRKETHLVPNEYTYSFMLEASARSLQWEYFEHVYQTMVLSGYQMDQTKHASMLIEASRAGKWSLLEHAFDAVLEDGEIPHPLFFTELLCHATAKGDFQRAITLINTVALASFQISEEEWTDLFEEHQDWLTQDNLHKLSDHLIECDYVSEPTVSNLSKSLKSRCGSSSSSAQPLLAVDVTTQSQGEKPEEDLLLQDTTMEDDNSANGEAWEFTETELETLGLEELEIDDDEESSDSDSLSVYDILKEWEESSKKE

SEQ ID NO: 5 - protein, 913 aa, Arabidopsis thaliana
MSLSHLLRRLCTTTTTTRSPLSISFLHQRIHNISLSPANEDPETTTGNNQDSEKYPNLNPIPNDPSQFQIPQNHTPPIPYPPIPHRTMAFSSAEEAAAERRRRKRRLRIEPPLHALRRDPSAPPPKRDPNAPRLPDSTSALVGQRLNLHNRVQSLIRASDLDAASKLARQSVFSNTRPTVFTCNAIIAAMYRAKRYSESISLFQYFFKQSNIVPNVVSYNQIINAHCDEGNVDEALEVYRHILANAPFAPSSVTYRHLTKGLVQAGRIGDAASLLREMLSKGQAADSTVYNNLIRGYLDLGDFDKAVEFFDELKSKCTVYDGIVNATFMEYWFEKGNDKEAMESYRSLLDKKFRMHPPTGNVLLEVFLKFGKKDEAWALFNEMLDNHAPPNILSVNSDTVGIMVNECFKMGEFSEAINTFKKVGSKVTSKPFVMDYLGYCNIVTRFCEQGMLTEAERFFAEGVSRSLPADAPSHRAMIDAYLKAERIDDAVKMLDRMVDVNLRVVADFGARVFGELIKNGKLTESAEVLTKMGEREPKPDPSIYDVVVRGLCDGDALDQAKDIVGEMIRHNVGVTTVLREFIIEVFEKAGRREEIEKILNSVARPVRNAGQSGNTPPRVPAVFGTTPAAPQQPRDRAPWTSQGVVHSNSGWANGTAGQTAGGAYKANNGQNPSWSNTSDNQQQQSWSNQTAGQQPPSWSRQAPGYQQQQSWSQQSGWSSPSGHQQSWTNQTAGQQQPWANQTPGQQQQWANQTPGQQQQLANQTPGQQQQWANQTPGQQQQWANQNNGHQQPWANQNTGHQQSWANQTPSQQQPWANQTTGQQQGWGNQTTGQQQQWANQTAGQQSGWTAQQQWSNQTASHQQSQWLNPVPGEVANQTPWSNSVDSHLPQQQEPGPSHECQETQEKKVVELRN

SEQ ID NO: 6 - protein, 196 aa, Flabovacterium okeianocoites
ALVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 7 - protein, 35 aa, Arabidopsis thaliana
NVYICNSILSCLVKNGKLDSCIKLFDQMKRDGLKP

SEQ ID NO: 8 - protein, 36 aa, Arabidopsis thaliana
DVVTYNTLLAGCIKVKNGYPKAIELIGELPHNGIQM

SEQ ID NO: 9 - protein, 35 aa, Arabidopsis thaliana
DSVMYGTVLAICASNGRSEEAENFIQQMKVEGHSP

SEQ ID NO: 10 - protein, 35 aa, Arabidopsis thaliana
NIYHYSSLLNSYSWKGDYKKADELMTEMKSIGLVP

SEQ ID NO: 11 - protein, 35 aa, Arabidopsis thaliana
NKVMMTTLLKVYIKGGLFDRSRELLSELESAGYAE

SEQ ID NO: 12 - protein, 35 aa, Arabidopsis thaliana
NEMPYCMLMDGLSKAGKLEEARSIFDDMKGKGVRS

SEQ ID NO: 13 - protein, 31 aa, Arabidopsis thaliana
DGYANSIMISALCRSKRFKEAKELSRDSETT

SEQ ID NO: 14 - protein, 35 aa, Arabidopsis thaliana
DLVMLNTMLCAYCRAGEMESVMRMMKKMDEQAVSP

SEQ ID NO: 15 - protein, 35 aa, Arabidopsis thaliana
DYNTFHILIKYFIKEKLHLLAYQTTLDMHSKGHRL

SEQ ID NO: 16 - protein, 36 aa, Arabidopsis thaliana
DVNLYNHYLRANLMMGASAGDMLDLVAPMEEFSVEP

SEQ ID NO: 17 - protein, 35 aa, Arabidopsis thaliana
NTASYNLVLKAMYQARETEAAMKLLERMLLLGKDS

SEQ ID NO: 18 - protein, 35 aa, Arabidopsis thaliana
DDESYDLVIGMHFGVGKNDEAMKVMDTALKSGYML

SEQ ID NO: 19 - protein, 36 aa, Arabidopsis thaliana
SVAALNCIILGCANTWDLDRAYQTFEAISASFGLTP

SEQ ID NO: 20 - protein, 35 aa, Arabidopsis thaliana
NIDSYNALLYAFGKVKKTFEATNVFEHLVSIGVKP

SEQ ID NO: 21 - protein, 35 aa, Arabidopsis thaliana
DSRTYSLLVDAHLINRDPKSALTVVDDMIKAGFEP

SEQ ID NO: 22 - protein, 35 aa, Arabidopsis thaliana
GEVVYRTLLANCVLKHHVNKAEDIFNKMKELKFPT

SEQ ID NO: 23 - protein, 34 aa, Arabidopsis thaliana
SVFACNQLLLLYSMHDRKKISDVLLLMERENIKP

SEQ ID NO: 24 - protein, 35 aa, Arabidopsis thaliana
SRATYHFLINSKGLAGDITGMEKIVETIKEEGIEL

SEQ ID NO: 25 - protein, 35 aa, Arabidopsis thaliana
DPELQSILAKYYIRAGLKERAQDLMKEIEGKGLQQ

SEQ ID NO: 26 - protein, 31 aa, Arabidopsis thaliana
TPWVCRSLLPLYADIGDSDNVRRLSRFVDQN

SEQ ID NO: 27 - protein, 31 aa, Arabidopsis thaliana
RYDNCISAIKAWGKLKEVEEAEAVFERLVEK

SEQ ID NO: 28 - protein, 35 aa, Arabidopsis thaliana
PMMPYFALMEIYTENKMLAKGRDLVKRMGNAGIAI

SEQ ID NO: 29 - protein, 36 aa, Arabidopsis thaliana
GPSTWHALVKLYIKAGEVGKAELILNRATKDNKMRP

SEQ ID NO: 30 - protein, 35 aa, Arabidopsis thaliana
MFTTYMAILEEYAKRGDVHNTEKVFMKMKRASYAA

SEQ ID NO: 31 - protein, 35 aa, Arabidopsis thaliana
SEIDFLMLITAYGKLGNFNGAERVLSVLSKMGSTP

SEQ ID NO: 32 - protein, 35 aa, Arabidopsis thaliana
NVISYTALMESYGRGGKCNNAEAIFRRMQSSGPEP

SEQ ID NO: 33 - protein, 35 aa, Arabidopsis thaliana
SAITYQIILKTFVEGDKFKEAEEVFETLLDEKKSP

SEQ ID NO: 34 - protein, 35 aa, Arabidopsis thaliana
DQKMYHMMIYMYKKAGNYEKARKVFSSMVGKGVPQ

SEQ ID NO: 35 - protein, 32 aa, Arabidopsis thaliana
STVTYNSLMSFETSYKEVSKIYDQMQRSDIQP

SEQ ID NO: 36 - protein, 35 aa, Arabidopsis thaliana
DVVSYALLIKAYGRARREEEALSVFEEMLDAGVRP

SEQ ID NO: 37 - protein, 35 aa, Arabidopsis thaliana
THKAYNILLDAFAISGMVEQAKTVFKSMRRDRIFP

SEQ ID NO: 38 - protein, 35 aa, Arabidopsis thaliana
DLWSYTTMLSAYVNASDMEGAEKFFKRIKVDGFEP

SEQ ID NO: 39 - protein, 35 aa, Arabidopsis thaliana
NIVTYGTLIKGYAKANDVEKMMEVYEKMRLSGIKA

SEQ ID NO: 40 - protein, 35 aa, Arabidopsis thaliana
NQTILTTIMDASGRCKNFGSALGWYKEMESCGVPP

SEQ ID NO: 41 - protein, 35 aa, Arabidopsis thaliana
NTIVMNSVLEACVHCGNIDLALRMFHEMAEPGGIG

SEQ ID NO: 42 - protein, 31 aa, Arabidopsis thaliana
DSISYATILKGLGKARRIDEAFQMLETIEYG

SEQ ID NO: 43 - protein, 31 aa, Arabidopsis thaliana
SSSLIYGLLDALINAGDLRRANGLLARYDIL

SEQ ID NO: 44 - protein, 35 aa, Arabidopsis thaliana
SVLIYNLLMKGYVNSESPQAAINLLDEMLRLRLEP

SEQ ID NO: 45 - protein, 31 aa, Arabidopsis thaliana
DRLTYNTLIHACIKCGDLDAAMKFFNDMKEK

SEQ ID NO: 46 - protein, 31 aa, Arabidopsis thaliana
DVVTYTTLVKGFGDATDLLSLQEIFLEMKLC

SEQ ID NO: 47 - protein, 36 aa, Arabidopsis thaliana
DRTAFTAVVDAMLKCGSTSGALCVFGEILKRSGANE

SEQ ID NO: 48 - protein, 35 aa, Arabidopsis thaliana
KPHLYLSMMRAFAVQGDYGMVRNLYLRLWPDSSGS

SEQ ID NO: 49 - protein, 36 aa, Arabidopsis thaliana
QQEADNLLMEAALNDGQLDEALGILLSIVRRWKTIP

SEQ ID NO: 50 - protein, 35 aa, Arabidopsis thaliana
CLSIHSSIMRDLCLQGKLDAALWLRKKMIYSGVIP

SEQ ID NO: 51 - protein, 35 aa, Arabidopsis thaliana
GLITHNHLLNGLCKAGYIEKADGLVREMREMGPSP

SEQ ID NO: 52 - protein, 35 aa, Arabidopsis thaliana
NCVSYNTLIKGLCSVNNVDKALYLFNTMNKYGIRP

SEQ ID NO: 53 - protein, 32 aa, Arabidopsis thaliana
NRVTCNIIVHALCQKGVIGNNNKKLLEEILDS

SEQ ID NO: 54 - protein, 35 aa, Arabidopsis thaliana
DIVICTILMDSCFKNGNVVQALEVWKEMSQKNVPA

SEQ ID NO: 55 - protein, 35 aa, Arabidopsis thaliana
DSVVYNVIIRGLCSSGNMVAAYGFMCDMVKRGVNP

SEQ ID NO: 56 - protein, 35 aa, Arabidopsis thaliana
DVFTYNTLISALCKEGKFDEACDLHGTMQNGGVAP

SEQ ID NO: 57 - protein, 35 aa, Arabidopsis thaliana
DQISYKVIIQGLCIHGDVNRANEFLLSMLKSSLLP

SEQ ID NO: 58 - protein, 35 aa, Arabidopsis thaliana
EVLLWNVVIDGYGRYGDTSSALSVLNLMLSYGVKP

SEQ ID NO: 59 - protein, 35 aa, Arabidopsis thaliana
NVYTNNALIHGYVKGGRLIDAWWVKNEMRSTKIHP

SEQ ID NO: 60 - protein, 35 aa, Arabidopsis thaliana
DTTTYNLLLGAACTLGHLRLAFQLYDEMLRRGCQP

SEQ ID NO: 61 - protein, 35 aa, Arabidopsis thaliana
DIITYTELVRGLCWKGRLKKAESLLSRIQATGITI

SEQ ID NO: 62 - protein, 31 aa, Arabidopsis thaliana
SRFVYTKLLSVLGFARRPQEALQIFNQMLGD

SEQ ID NO: 63 - protein, 35 aa, Arabidopsis thaliana
DMAAYHCIAVTLGQAGLLKELLKVIERMRQKPTKL

SEQ ID NO: 64 - protein, 35 aa, Arabidopsis thaliana
DLVVYNAILNACVPTLQWKAVSWVFVELRKNGLRP

SEQ ID NO: 65 - protein, 35 aa, Arabidopsis thaliana
NGATYGLAMEVMLESGKFDRVHDFFRKMKSSGEAP

SEQ ID NO: 66 - protein, 35 aa, Arabidopsis thaliana
KAITYKVLVRALWREGKIEEAVEAVRDMEQKGVIG

SEQ ID NO: 67 - protein, 36 aa, Arabidopsis thaliana
TGSVYYELACCLCNNGRWCDAMLEVGRMKRLENCRP

SEQ ID NO: 68 - protein, 31 aa, Arabidopsis thaliana
LEITFTGLIAASLNGGHVDDCMAIFQYMKDK

SEQ ID NO: 69 - protein, 31 aa, Arabidopsis thaliana
NIGTANMMLKVYGRNDMFSEAKELFEEIVSR

SEQ ID NO: 70 - protein, 35 aa, Arabidopsis thaliana
NEYTYSFMLEASARSLQWEYFEHVYQTMVLSGYQM

SEQ ID NO: 71 - protein, 35 aa, Arabidopsis thaliana
DQTKHASMLIEASRAGKWSLLEHAFDAVLEDGEIP

SEQ ID NO: 72 - protein, 35 aa, Arabidopsis thaliana
QIVDYAPLVQTLSQRRLPDVAHEIFLQTKSVNLLP

SEQ ID NO: 73 - protein, 35 aa, Arabidopsis thaliana
NYRTLCALMLCFAENGFVLRARTIWDEIINSCFVP

SEQ ID NO: 74 - protein, 35 aa, Arabidopsis thaliana
DVFVVSKLISAYEQFGCFDEVAKITKDVAARHSKL

SEQ ID NO: 75 - protein, 35 aa, Arabidopsis thaliana
LPVVSSLAISCFGKNGQLELMEGVIEEMDSKGVLL

SEQ ID NO: 76 - protein, 35 aa, Arabidopsis thaliana
EAETANVIVRYYSFFGSLDKMEKAYGRVKKFGIVI

SEQ ID NO: 77 - protein, 35 aa, Arabidopsis thaliana
EEEEIRAVVLAYLKQRKFYRLREFLSDVGLGRRNL

SEQ ID NO: 78 - protein, 35 aa, Arabidopsis thaliana
GNMLWNSVLLSYAADFKMKSLQREFIGMLDAGFSP

SEQ ID NO: 79 - protein, 35 aa, Arabidopsis thaliana
DLTTFNIRALAFSRMALFWDLHLTLEHMRRLNIVP

SEQ ID NO: 80 - protein, 35 aa, Arabidopsis thaliana
DLVTFGCVVDAYMDKRLARNLEFVYNRMNLDDSPL

SEQ ID NO: 81 - protein, 35 aa, Arabidopsis thaliana
TPLTYNALIGACARNNDIEKALNLIAKMRQDGYQS

SEQ ID NO: 82 - protein, 37 aa, Arabidopsis thaliana
DFVNYSLVIQSLTRSNKIDSVMLLRLYKEIERDKLEL

SEQ ID NO: 83 - protein, 35 aa, Arabidopsis thaliana
DVQLVNDIIMGFAKSGDPSKALQLLGMAQATGLSA

SEQ ID NO: 84 - protein, 35 aa, Arabidopsis thaliana
KTATLVSIISALADSGRTLEAEALFEELRQSGIKP

SEQ ID NO: 85 - protein, 35 aa, Arabidopsis thaliana
RTRAYNALLKGYVKTGPLKDAESMVSEMEKRGVSP

SEQ ID NO: 86 - protein, 35 aa, Arabidopsis thaliana
DEHTYSLLIDAYVNAGRWESARIVLKEMEAGDVQP

SEQ ID NO: 87 - protein, 35 aa, Arabidopsis thaliana
NSFVFSRLLAGFRDRGEWQKTFQVLKEMKSIGVKP

SEQ ID NO: 88 - protein, 35 aa, Arabidopsis thaliana
DRQFYNVVIDTFGKFNCLDHAMTTFDRMLSEGIEP

SEQ ID NO: 89 - protein, 35 aa, Arabidopsis thaliana
DRVTWNTLIDCHCKHGRHIVAEEMFEAMERRGCLP

SEQ ID NO: 90 - protein, 35 aa, Arabidopsis thaliana
CATTYNIMINSYGDQERWDDMKRLLGKMKSQGILP

SEQ ID NO: 91 - protein, 35 aa, Arabidopsis thaliana
NVVTHTTLVDVYGKSGRFNDAIECLEEMKSVGLKP

SEQ ID NO: 92 - protein, 35 aa, Arabidopsis thaliana
SSTMYNALINAYAQRGLSEQAVNAFRVMTSDGLKP

SEQ ID NO: 93 - protein, 35 aa, Arabidopsis thaliana
SLLALNSLINAFGEDRRDAEAFAVLQYMKENGVKP

SEQ ID NO: 94 - protein, 35 aa, Arabidopsis thaliana
DVVTYTTLMKALIRVDKFQKVPVVYEEMIMSGCKP

SEQ ID NO: 95 - protein, 35 aa, Arabidopsis thaliana
SLVDFSRFFSAIARTKQFNLVLDFCKQLELNGIAH

SEQ ID NO: 96 - protein, 35 aa, Arabidopsis thaliana
NIYTLNIMINCFCRCCKTCFAYSVLGKVMKLGYEP

SEQ ID NO: 97 - protein, 35 aa, Arabidopsis thaliana
DTTTFNTLIKGLFLEGKVSEAVVLVDRMVENGCQP

SEQ ID NO: 98 - protein, 35 aa, Arabidopsis thaliana
DVVTYNSIVNGICRSGDTSLALDLLRKMEERNVKA

SEQ ID NO: 99 - protein, 35 aa, Arabidopsis thaliana
DVFTYSTIIDSLCRDGCIDAAISLFKEMETKGIKS

SEQ ID NO: 100 - protein, 35 aa, Arabidopsis thaliana
SVVTYNSLVRGLCKAGKWNDGALLLKDMVSREIVP

SEQ ID NO: 101 - protein, 35 aa, Arabidopsis thaliana
NVITFNVLLDVFVKEGKLQEANELYKEMITRGISP

SEQ ID NO: 102 - protein, 35 aa, Arabidopsis thaliana
NIITYNTLMDGYCMQNRLSEANNMLDLMVRNKCSP

SEQ ID NO: 103 - protein, 35 aa, Arabidopsis thaliana
DIVTFTSLIKGYCMVKRVDDGMKVFRNISKRGLVA

SEQ ID NO: 104 - protein, 35 aa, Arabidopsis thaliana
NAVTYSILVQGFCQSGKIKLAEELFQEMVSHGVLP

SEQ ID NO: 105 - protein, 35 aa, Arabidopsis thaliana
DVMTYGILLDGLCDNGKLEKALEIFEDLQKSKMDL

SEQ ID NO: 106 - protein, 35 aa, Arabidopsis thaliana
GIVMYTTIIEGMCKGGKVEDAWNLFCSLPCKGVKP

SEQ ID NO: 107 - protein, 35 aa, Arabidopsis thaliana
NVMTYTVMISGLCKKGSLSEANILLRKMEEDGNAP

SEQ ID NO: 108 - protein, 35 aa, Arabidopsis thaliana
NDCTYNTLIRAHLRDGDLTASAKLIEEMKSCGFSA

SEQ ID NO: 109 - protein, 35 aa, Arabidopsis thaliana
TDYTVMRLIHFLGKLGNWRRVLQVIEWLQRQDRYK

SEQ ID NO: 110 - protein, 31 aa, Arabidopsis thaliana
IRIIYTTALNVLGKSRRPVEALNVFHAMLLQ

SEQ ID NO: 111 - protein, 31 aa, Arabidopsis thaliana
DMVAYRSIAVTLGQAGHIKELFYVIDTMRSP

SEQ ID NO: 112 - protein, 35 aa, Arabidopsis thaliana
DVVVYNAVLNACVQRKQWEGAFWVLQQLKQRGQKP

SEQ ID NO: 113 - protein, 31 aa, Arabidopsis thaliana
SPVTYGLIMEVMLACEKYNLVHEFFRKMQKS

SEQ ID NO: 114 - protein, 35 aa, Arabidopsis thaliana
NALAYRVLVNTLWKEGKSDEAVHTVEDMESRGIVG

SEQ ID NO: 115 - protein, 31 aa, Arabidopsis thaliana
LVVTYTGLIQACVDSGNIKNAAYIFDQMKKV

SEQ ID NO: 116 - protein, 35 aa, Arabidopsis thaliana
NLVTCNIMLKAYLQGGLFEEARELFQKMSEDGNHI

SEQ ID NO: 117 - protein, 35 aa, Arabidopsis thaliana
DTYTFNTMLDTCAEQEKWDDFGYAYREMLRHGYHF

SEQ ID NO: 118 - protein, 35 aa, Arabidopsis thaliana
NAKRHLRMVLEASRAGKEEVMEATWEHMRRSNRIP

SEQ ID NO: 119 - protein, 35 aa, Arabidopsis thaliana
DVICFNLLIDAYGQKFQYKEAESLYVQLLESRYVP

SEQ ID NO: 120 - protein, 35 aa, Arabidopsis thaliana
TEDTYALLIKAYCMAGLIERAEVVLVEMQNHHVSP

SEQ ID NO: 121 - protein, 36 aa, Arabidopsis thaliana
GVTVYNAYIEGLMKRKGNTEEAIDVFQRMKRDRCKP

SEQ ID NO: 122 - protein, 35 aa, Arabidopsis thaliana
TTETYNLMINLYGKASKSYMSWKLYCEMRSHQCKP

SEQ ID NO: 123 - protein, 35 aa, Arabidopsis thaliana
NICTYTALVNAFAREGLCEKAEEIFEQLQEDGLEP

SEQ ID NO: 124 - protein, 35 aa, Arabidopsis thaliana
DVYVYNALMESYSRAGYPYGAAEIFSLMQHMGCEP

SEQ ID NO: 125 - protein, 35 aa, Arabidopsis thaliana
DRASYNIMVDAYGRAGLHSDAEAVFEEMKRLGIAP

SEQ ID NO: 126 - protein, 35 aa, Arabidopsis thaliana
TMKSHMLLLSAYSKARDVTKCEAIVKEMSENGVEP

SEQ ID NO: 127 - protein, 35 aa, Arabidopsis thaliana
DTFVLNSMLNLYGRLGQFTKMEKILAEMENGPCTA

SEQ ID NO: 128 - protein, 35 aa, Arabidopsis thaliana
DISTYNILINIYGKAGFLERIEELFVELKEKNFRP

SEQ ID NO: 129 - protein, 35 aa, Arabidopsis thaliana
DVVTWTSRIGAYSRKKLYVKCLEVFEEMIDSGCAP

SEQ ID NO: 130 - protein, 31 aa, Arabidopsis thaliana
DGGTAKVLLSACSSEEQVEQVTSVLRTMHKG

SEQ ID NO: 131 - protein, 31 aa, Arabidopsis thaliana
ARKNFPVLIRELSRRGCIELCVNVFKWMKIQ

SEQ ID NO: 132 - protein, 35 aa, Arabidopsis thaliana
RNDIYNMMIRLHARHNWVDQARGLFFEMQKWSCKP

SEQ ID NO: 133 - protein, 35 aa, Arabidopsis thaliana
DAETYDALINAHGRAGQWRWAMNLMDDMLRAAIAP

SEQ ID NO: 134 - protein, 35 aa, Arabidopsis thaliana
SRSTYNNLINACGSSGNWREALEVCKKMTDNGVGP

SEQ ID NO: 135 - protein, 35 aa, Arabidopsis thaliana
DLVTHNIVLSAYKSGRQYSKALSYFELMKGAKVRP

SEQ ID NO: 136 - protein, 35 aa, Arabidopsis thaliana
DTTTFNIIIYCLSKLGQSSQALDLFNSMREKRAEC

SEQ ID NO: 137 - protein, 35 aa, Arabidopsis thaliana
DVVTFTSIMHLYSVKGEIENCRAVFEAMVAEGLKP

SEQ ID NO: 138 - protein, 35 aa, Arabidopsis thaliana
NIVSYNALMGAYAVHGMSGTALSVLGDIKQNGIIP

SEQ ID NO: 139 - protein, 35 aa, Arabidopsis thaliana
DVVSYTCLLNSYGRSRQPGKAKEVFLMMRKERRKP

SEQ ID NO: 140 - protein, 35 aa, Arabidopsis thaliana
NVVTYNALIDAYGSNGFLAEAVEIFRQMEQDGIKP

SEQ ID NO: 141 - protein, 35 aa, Arabidopsis thaliana
NVVSVCTLLAACSRSKKKVNVDTVLSAAQSRGINL

SEQ ID NO: 142 - protein, 35 aa, Arabidopsis thaliana
NTAAYNSAIGSYINAAELEKAIALYQSMRKKKVKA

SEQ ID NO: 143 - protein, 35 aa, Arabidopsis thaliana
DSVTFTILISGSCRMSKYPEAISYLKEMEDLSIPL

SEQ ID NO: 144 - protein, 35 aa, Arabidopsis thaliana
TKEVYSSVLCAYSKQGQVTEAESIFNQMKMAGCEP

SEQ ID NO: 145 - protein, 35 aa, Arabidopsis thaliana
DVIAYTSMLHAYNASEKWGKACELFLEMEANGIEP

SEQ ID NO: 146 - protein, 35 aa, Arabidopsis thaliana
DSIACSALMRAFNKGGQPSNVFVLMDLMREKEIPF

SEQ ID NO: 147 - protein, 31 aa, Arabidopsis thaliana
TGAVFFEIFSACNTLQEWKRAIDLIQMMDPY

SEQ ID NO: 148 - protein, 35 aa, Arabidopsis thaliana
SIGLTNQMLHLFGKSGKVEAMMKLFYKIIASGVGI

SEQ ID NO: 149 - protein, 35 aa, Arabidopsis thaliana
NLKTYAILLEHLLAVGNWRKYIEVLEWMSGAGIQP

SEQ ID NO: 150 - protein, 35 aa, Arabidopsis thaliana
DRSFYHTMMKISRDSGSDSKAEKLLQMMKNAGIEP

SEQ ID NO: 151 - protein, 35 aa, Arabidopsis thaliana
TLATMHLLMVSYSSSGNPQEAEKVLSNLKDTEVEL

SEQ ID NO: 152 - protein, 35 aa, Arabidopsis thaliana
TTLPYSSVIDAYLRSKDYNSGIERLLEMKKEGLEP

SEQ ID NO: 153 - protein, 35 aa, Arabidopsis thaliana
RVQVYNAMMGVYSRSGKFSKAQELVDAMRQRGCVP

SEQ ID NO: 154 - protein, 37 aa, Arabidopsis thaliana
DLISFNTLINARLKSGGLTPNLAVELLDMVRNSGLRP

SEQ ID NO: 155 - protein, 35 aa, Arabidopsis thaliana
DAITYNTLLSACSRDSNLDGAVKVFEDMEAHRCQP

SEQ ID NO: 156 - protein, 35 aa, Arabidopsis thaliana
DLWTYNAMISVYGRCGLAAEAERLFMELELKGFFP

SEQ ID NO: 157 - protein, 35 aa, Arabidopsis thaliana
DAVTYNSLLYAFARERNTEKVKEVYQQMQKMGFGK

SEQ ID NO: 158 - protein, 31 aa, Arabidopsis thaliana
DEMTYNTIIHMYGKQGQLDLALQLYKDMKGL

SEQ ID NO: 159 - protein, 35 aa, Arabidopsis thaliana
DAITYTVLIDSLGKANRTVEAAALMSEMLDVGIKP

SEQ ID NO: 160 - protein, 35 aa, Arabidopsis thaliana
TLQTYSALICGYAKAGKREEAEDTFSCMLRSGTKP

SEQ ID NO: 161 - protein, 35 aa, Arabidopsis thaliana
DNLAYSVMLDVLLRGNETRKAWGLYRDMISDGHTP

SEQ ID NO: 162 - protein, 31 aa, Arabidopsis thaliana
SYTLYELMILGLMKENRSDDIQKTIRDMEEL

SEQ ID NO: 163 - protein, 31 aa, Arabidopsis thaliana
ENDTLLSILGSYSSSGRHSEAFELLEFLKEH

SEQ ID NO: 164 - protein, 36 aa, Arabidopsis thaliana
KRLITEALIVLHCKVNNLSAALDEYFADPCVHGWCF

SEQ ID NO: 165 - protein, 35 aa, Arabidopsis thaliana
SSTMYETLLHCCVANEHYAEASQVFSDLRLSGCEA

SEQ ID NO: 166 - protein, 35 aa, Arabidopsis thaliana
SESVCKSMVVVYCKLGFPETAHQVVNQAETKGFHF

SEQ ID NO: 167 - protein, 35 aa, Arabidopsis thaliana
CSPMYTDIIEAYGKQKLWQKAESVVGNLRQSGRTP

SEQ ID NO: 168 - protein, 35 aa, Arabidopsis thaliana
DLKTWNSLMSAYAQCGCYERARAIFNTMMRDGPSP

SEQ ID NO: 169 - protein, 35 aa, Arabidopsis thaliana
TVESINILLHALCVDGRLEELYVVVEELQDMGFKI

SEQ ID NO: 170 - protein, 35 aa, Arabidopsis thaliana
SKSSILLMLDAFARAGNIFEVKKIYSSMKAAGYLP

SEQ ID NO: 171 - protein, 35 aa, Arabidopsis thaliana
TIRLYRMMIELLCKGKRVRDAEIMVSEMEEANFKV

SEQ ID NO: 172 - protein, 35 aa, Arabidopsis thaliana
ELAIWNSMLKMYTAIEDYKKTVQVYQRIKETGLEP

SEQ ID NO: 173 - protein, 35 aa, Arabidopsis thaliana
DETTYNTLIIMYCRDRRPEEGYLLMQQMRNLGLDP

SEQ ID NO: 174 - protein, 35 aa, Arabidopsis thaliana
KLDTYKSLISAFGKQKCLEQAEQLFEELLSKGLKL

SEQ ID NO: 175 - protein, 35 aa, Arabidopsis thaliana
RCKTYTKLFKVLGNCKQPDQASLLFEVMLSEGLKP

SEQ ID NO: 176 - protein, 31 aa, Arabidopsis thaliana
TIDVYTSLISVYGKSELLDKAFSTLEYMKSV

SEQ ID NO: 177 - protein, 35 aa, Arabidopsis thaliana
DVFTFTVLISCCCKLGRFDLVKSIVLEMSYLGVGC

SEQ ID NO: 178 - protein, 35 aa, Arabidopsis thaliana
STVTYNTIIDGYGKAGMFEEMESVLADMIEDGDSL

SEQ ID NO: 179 - protein, 35 aa, Arabidopsis thaliana
DVCTLNSIIGSYGNGRNMRKMESWYSRFQLMGVQP

SEQ ID NO: 180 - protein, 35 aa, Arabidopsis thaliana
DITTFNILILSFGKAGMYKKMCSVMDFMEKRFFSL

SEQ ID NO: 181 - protein, 35 aa, Arabidopsis thaliana
TTVTYNIVIETFGKAGRIEKMDDVFRKMKYQGVKP

SEQ ID NO: 182 - protein, 35 aa, Arabidopsis thaliana
NSITYCSLVNAYSKAGLVVKIDSVLRQIVNSDVVL

SEQ ID NO: 183 - protein, 35 aa, Arabidopsis thaliana
DTPFFNCIINAYGQAGDLATMKELYIQMEERKCKP

SEQ ID NO: 184 - protein, 35 aa, Arabidopsis thaliana
DKITFATMIKTYTAHGIFDAVQELEKQMISSGENL

SEQ ID NO: 185 - protein, 35 aa, Arabidopsis thaliana
LSVSLSLVLEYYALKGSHHNGLEVFGFMRRLRLSP

SEQ ID NO: 186 - protein, 35 aa, Arabidopsis thaliana
SQSAYNSLLGSLVKENQFRVALCLYSAMVRNGIVS

SEQ ID NO: 187 - protein, 26 aa, Arabidopsis thaliana
DELTWDLIAQILCEQGRSKSVFKLME

SEQ ID NO: 188 - protein, 35 aa, Arabidopsis thaliana
SCKIYTNLVECYSRNGEFDAVFSLIHEMDDKKLEL

SEQ ID NO: 189 - protein, 35 aa, Arabidopsis thaliana
SFCSYGCVLDDACRLGDAEFIDKVLCLMVEKKFVT

SEQ ID NO: 190 - protein, 35 aa, Arabidopsis thaliana
DSAVNDKIIERLCDMGKTFASEMLFRKACNGETVR

SEQ ID NO: 191 - protein, 35 aa, Arabidopsis thaliana
WDSTYGCMLKALSRKKRTKEAVDVYRMICRKGITV

SEQ ID NO: 192 - protein, 36 aa, Arabidopsis thaliana
DESCYIEFANALCRDDNSSEEEEELLVDVIKRGFVP

SEQ ID NO: 193 - protein, 35 aa, Arabidopsis thaliana
CTHKLSEVLASMCRKRRWKSAEKLLDSVMEMEVYF

SEQ ID NO: 194 - protein, 35 aa, Arabidopsis thaliana
NVGIYVKLIVMLGKCKQPEKAHELFQEMINEGCVV

SEQ ID NO: 195 - protein, 31 aa, Arabidopsis thaliana
NHEVYTALVSAYSRSGRFDAAFTLLERMKSS

SEQ ID NO: 196 - protein, 35 aa, Arabidopsis thaliana
DVHTYSILIKSFLQVFAFDKVQDLLSDMRRQGIRP

SEQ ID NO: 197 - protein, 36 aa, Arabidopsis thaliana
NTITYNTLIDAYGKAKMFVEMESTLIQMLGEDDCKP

SEQ ID NO: 198 - protein, 35 aa, Arabidopsis thaliana
DSWTMNSTLRAFGGNGQIEMMENCYEKFQSSGIEP

SEQ ID NO: 199 - protein, 35 aa, Arabidopsis thaliana
NIRTFNILLDSYGKSGNYKKMSAVMEYMQKYHYSW

SEQ ID NO: 200 - protein, 35 aa, Arabidopsis thaliana
TIVTYNVVIDAFGRAGDLKQMEYLFRLMQSERIFP

SEQ ID NO: 201 - protein, 35 aa, Arabidopsis thaliana
SCVTLCSLVRAYGRASKADKIGGVLRFIENSDIRL

SEQ ID NO: 202 - protein, 35 aa, Arabidopsis thaliana
DLVFFNCLVDAYGRMEKFAEMKGVLELMEKKGFKP

SEQ ID NO: 203 - protein, 35 aa, Arabidopsis thaliana
DKITYRTMVKAYRISGMTTHVKELHGVVESVGEAQ

SEQ ID NO: 204 - protein, 35 aa, Arabidopsis thaliana
DVRLYNAAISGLSASQRYDDAWEVYEAMDKINVYP

SEQ ID NO: 205 - protein, 36 aa, Arabidopsis thaliana
DNVTCAILITTLRKAGRSAKEVWEIFEKMSEKGVKW

SEQ ID NO: 206 - protein, 35 aa, Arabidopsis thaliana
SQDVFGGLVKSFCDEGLKEEALVIQTEMEKKGIRS

SEQ ID NO: 207 - protein, 35 aa, Arabidopsis thaliana
NTIVYNTLMDAYNKSNHIEEVEGLFTEMRDKGLKP

SEQ ID NO: 208 - protein, 35 aa, Arabidopsis thaliana
SAATYNILMDAYARRMQPDIVETLLREMEDLGLEP

SEQ ID NO: 209 - protein, 36 aa, Arabidopsis thaliana
NVKSYTCLISAYGRTKKMSDMAADAFLRMKKVGLKP

SEQ ID NO: 210 - protein, 35 aa, Arabidopsis thaliana
SSHSYTALIHAYSVSGWHEKAYASFEEMCKEGIKP

SEQ ID NO: 211 - protein, 35 aa, Arabidopsis thaliana
SVETYTSVLDAFRRSGDTGKLMEIWKLMLREKIKG

SEQ ID NO: 212 - protein, 35 aa, Arabidopsis thaliana
TRITYNTLLDGFAKQGLYIEARDVVSEFSKMGLQP

SEQ ID NO: 213 - protein, 35 aa, Arabidopsis thaliana
SVMTYNMLMNAYARGGQDAKLPQLLKEMAALNLKP

SEQ ID NO: 214 - protein, 35 aa, Arabidopsis thaliana
DSITYSTMIYAFVRVRDFKRAFFYHKMMVKSGQVP

SEQ ID NO: 215 - protein, 31 aa, Arabidopsis thaliana
TSDSFEKTLHILARMRYFDQAWALMAEVRKD

SEQ ID NO: 216 - protein, 35 aa, Arabidopsis thaliana
SFKSMSILLCKIAKFGSYEETLEAFVKMEKEIFRK

SEQ ID NO: 217 - protein, 31 aa, Arabidopsis thaliana
GVDEFNILLRAFCTEREMKEARSIFEKLHSR

SEQ ID NO: 218 - protein, 35 aa, Arabidopsis thaliana
DVKTMNILLLGFKEAGDVTATELFYHEMVKRGFKP

SEQ ID NO: 219 - protein, 35 aa, Arabidopsis thaliana
NSVTYGIRIDGFCKKRNFGEALRLFEDMDRLDFDI

SEQ ID NO: 220 - protein, 35 aa, Arabidopsis thaliana
TVQILTTLIHGSGVARNKIKARQLFDEISKRGLTP

SEQ ID NO: 221 - protein, 35 aa, Arabidopsis thaliana
DCGAYNALMSSLMKCGDVSGAIKVMKEMEEKGIEP

SEQ ID NO: 222 - protein, 37 aa, Arabidopsis thaliana
DSVTFHSMFIGMMKSKEFGFNGVCEYYQKMKERSLVP

SEQ ID NO: 223 - protein, 35 aa, Arabidopsis thaliana
KTPTIVMLMKLFCHNGEVNLGLDLWKYMLEKGYCP

SEQ ID NO: 224 - protein, 32 aa, Arabidopsis thaliana
SPSLFDSVVNSLCKAREFEIAWSLVFDRVRSD

SEQ ID NO: 225 - protein, 31 aa, Arabidopsis thaliana
SADTFIVLIRRYARAGMVQQAIRAFEFARSY

SEQ ID NO: 226 - protein, 31 aa, Arabidopsis thaliana
ELRLLEVLLDALCKEGHVREASMYLERIGGT

SEQ ID NO: 227 - protein, 35 aa, Arabidopsis thaliana
SVRIFNILLNGWFRSRKLKQAEKLWEEMKAMNVKP

SEQ ID NO: 228 - protein, 35 aa, Arabidopsis thaliana
TVVTYGTLIEGYCRMRRVQIAMEVLEEMKMAEMEI

SEQ ID NO: 229 - protein, 35 aa, Arabidopsis thaliana
NFMVFNPIIDGLGEAGRLSEALGMMERFFVCESGP

SEQ ID NO: 230 - protein, 35 aa, Arabidopsis thaliana
TIVTYNSLVKNFCKAGDLPGASKILKMMMTRGVDP

SEQ ID NO: 231 - protein, 35 aa, Arabidopsis thaliana
TTTTYNHFFKYFSKHNKTEEGMNLYFKLIEAGHSP

SEQ ID NO: 232 - protein, 35 aa, Arabidopsis thaliana
DRLTYHLILKMLCEDGKLSLAMQVNKEMKNRGIDP

SEQ ID NO: 233 - protein, 35 aa, Arabidopsis thaliana
DLLTTTMLIHLLCRLEMLEEAFEEFDNAVRRGIIP

SEQ ID NO: 234 - protein, 35 aa, Arabidopsis thaliana
QYITFKMIDNGLRSKGMSDMAKRLSSLMSSLPHSK

SEQ ID NO: 235 - protein, 35 aa, Arabidopsis thaliana
TAPVYNALVDLIVRDDDEKVPEEFLQQIRDDDKEV

SEQ ID NO: 236 - protein, 35 aa, Arabidopsis thaliana
FGEFLNVLVRKHCRNGSFSIALEELGRLKDFRFRP

SEQ ID NO: 237 - protein, 35 aa, Arabidopsis thaliana
SRSTYNCLIQAFLKADRLDSASLIHREMSLANLRM

SEQ ID NO: 238 - protein, 31 aa, Arabidopsis thaliana
DGFTLRCFAYSLCKVGKWREALTLVETENFV

SEQ ID NO: 239 - protein, 35 aa, Arabidopsis thaliana
DTVFYTKLISGLCEASLFEEAMDFLNRMRATSCLP

SEQ ID NO: 240 - protein, 35 aa, Arabidopsis thaliana
NVVTYSTLLCGCLNKKQLGRCKRVLNMMMMEGCYP

SEQ ID NO: 241 - protein, 35 aa, Arabidopsis thaliana
SPKIFNSLVHAYCTSGDHSYAYKLLKKMVKCGHMP

SEQ ID NO: 242 - protein, 41 aa, Arabidopsis thaliana
GYVVYNILIGSICGDKDSLNCDLLDLAEKAYSEMLAAGVVL

SEQ ID NO: 243 - protein, 35 aa, Arabidopsis thaliana
NKINVSSFTRCLCSAGKYEKAFSVIREMIGQGFIP

SEQ ID NO: 244 - protein, 35 aa, Arabidopsis thaliana
DTSTYSKVLNYLCNASKMELAFLLFEEMKRGGLVA

SEQ ID NO: 245 - protein, 35 aa, Arabidopsis thaliana
DVYTYTIMVDSFCKAGLIEQARKWFNEMREVGCTP

SEQ ID NO: 246 - protein, 35 aa, Arabidopsis thaliana
NVVTYTALIHAYLKAKKVSYANELFETMLSEGCLP

SEQ ID NO: 247 - protein, 35 aa, Arabidopsis thaliana
NIVTYSALIDGHCKAGQVEKACQIFERMCGSKDVP

SEQ ID NO: 248 - protein, 35 aa, Arabidopsis thaliana
NVVTYGALLDGFCKSHRVEEARKLLDAMSMEGCEP

SEQ ID NO: 249 - protein, 35 aa, Arabidopsis thaliana
NQIVYDALIDGLCKVGKLDEAQEVKTEMSEHGFPA

SEQ ID NO: 250 - protein, 35 aa, Arabidopsis thaliana
TLYTYSSLIDRYFKVKRQDLASKVLSKMLENSCAP

SEQ ID NO: 251 - protein, 35 aa, Arabidopsis thaliana
NVVIYTEMIDGLCKVGKTDEAYKLMQMMEEKGCQP

SEQ ID NO: 252 - protein, 35 aa, Arabidopsis thaliana
NVVTYTAMIDGFGMIGKIETCLELLERMGSKGVAP

SEQ ID NO: 253 - protein, 35 aa, Arabidopsis thaliana
NYVTYRVLIDHCCKNGALDVAHNLLEEMKQTHWPT

SEQ ID NO: 254 - protein, 35 aa, Arabidopsis thaliana
FLSVYRLLIDNLIKAQRLEMALRLLEEVATFSATL

SEQ ID NO: 255 - protein, 35 aa, Arabidopsis thaliana
YSSTYNSLIESLCLANKVETAFQLFSEMTKKGVIP

SEQ ID NO: 256 - protein, 35 aa, Arabidopsis thaliana
EMQSFCSLIKGLFRNSKISEALLLLDFISHMVCPL

SEQ ID NO: 257 - protein, 35 aa, Arabidopsis thaliana
DVRAYTTILHAYSRTGKYEKAIDLFERMKEMGPSP

SEQ ID NO: 258 - protein, 36 aa, Arabidopsis thaliana
TLVTYNVILDVFGKMGRSWRKILGVLDEMRSKGLKF

SEQ ID NO: 259 - protein, 35 aa, Arabidopsis thaliana
DEFTCSTVLSACAREGLLREAKEFFAELKSCGYEP

SEQ ID NO: 260 - protein, 35 aa, Arabidopsis thaliana
GTVTYNALLQVFGKAGVYTEALSVLKEMEENSCPA

SEQ ID NO: 261 - protein, 35 aa, Arabidopsis thaliana
DSVTYNELVAAYVRAGFSKEAAGVIEMMTKKGVMP

SEQ ID NO: 262 - protein, 35 aa, Arabidopsis thaliana
NAITYTTVIDAYGKAGKEDEALKLFYSMKEAGCVP

SEQ ID NO: 263 - protein, 35 aa, Arabidopsis thaliana
NTCTYNAVLSLLGKKSRSNEMIKMLCDMKSNGCSP

SEQ ID NO: 264 - protein, 35 aa, Arabidopsis thaliana
NRATWNTMLALCGNKGMDKFVNRVFREMKSCGFEP

SEQ ID NO: 265 - protein, 35 aa, Arabidopsis thaliana
DRDTFNTLISAYGRCGSEVDASKMYGEMTRAGFNA

SEQ ID NO: 266 - protein, 35 aa, Arabidopsis thaliana
CVTTYNALLNALARKGDWRSGENVISDMKSKGFKP

SEQ ID NO: 267 - protein, 35 aa, Arabidopsis thaliana
TETSYSLMLQCYAKGGNYLGIERIENRIKEGQIFP

SEQ ID NO: 268 - protein, 35 aa, Arabidopsis thaliana
SWMLLRTLLLANFKCRALAGSERAFTLFKKHGYKP

SEQ ID NO: 269 - protein, 35 aa, Arabidopsis thaliana
DMVIFNSMLSIFTRNNMYDQAEGILESIREDGLSP

SEQ ID NO: 270 - protein, 35 aa, Arabidopsis thaliana
DLVTYNSLMDMYVRRGECWKAEEILKTLEKSQLKP

SEQ ID NO: 271 - protein, 35 aa, Arabidopsis thaliana
DLVSYNTVIKGFCRRGLMQEAVRMLSEMTERGIRP

SEQ ID NO: 272 - protein, 35 aa, Arabidopsis thaliana
CIFTYNTFVSGYTAMGMFAEIEDVIECMAKNDCRP

SEQ ID NO: 273 - protein, 31 aa, Arabidopsis thaliana
NELTFKMVVDGYCRAGKYSEAMDFVSKIKTF

SEQ ID NO: 274 - protein, 35 aa, Arabidopsis thaliana
DTAAFNAVLNACANLGDTDKYWKLFEEMSEWDCEP

SEQ ID NO: 275 - protein, 35 aa, Arabidopsis thaliana
DVLTYNVMIKLCARVGRKELIVFVLERIIDKGIKV

SEQ ID NO: 276 - protein, 35 aa, Arabidopsis thaliana
CMTTMHSLVAAYVGFGDLRTAERIVQAMREKRRDL

SEQ ID NO: 277 - protein, 31 aa, Arabidopsis thaliana
DSRIYTTLMKGYMKNGRVADTARMLEAMRRQ

SEQ ID NO: 278 - protein, 35 aa, Arabidopsis thaliana
DEVTYTTVVSAFVNAGLMDRARQVLAEMARMGVPA

SEQ ID NO: 279 - protein, 36 aa, Arabidopsis thaliana
NRITYNVLLKGYCKQLQIDRAEDLLREMTEDAGIEP

SEQ ID NO: 280 - protein, 35 aa, Arabidopsis thaliana
DVVSYNIIIDGCILIDDSAGALAFFNEMRTRGIAP

SEQ ID NO: 281 - protein, 31 aa, Arabidopsis thaliana
TKISYTTLMKAFAMSGQPKLANRVFDEMMND

SEQ ID NO: 282 - protein, 35 aa, Arabidopsis thaliana
DLIAWNMLVEGYCRLGLIEDAQRVVSRMKENGFYP

SEQ ID NO: 283 - protein, 31 aa, Arabidopsis thaliana
NVATYGSLANGVSQARKPGDALLLWKEIKER

SEQ ID NO: 284 - protein, 35 aa, Artificial Sequence
  crPPR
VTYTTLISGLGKAGRLEEALELFEEMKEKGIVPNV

SEQ ID NO: 285 - protein, 35 aa, Artificial Sequence
  MODIFIED TYPE crPPR-1
VTYTTLISAYGKAGRLEEALELFEEMKEKGIVPNV

SEQ ID NO: 286 - protein, 35 aa, Artificial Sequence
  MODIFIED TYPE crPPR-2
VTYTTLISGLGKAGRLEKAEELFEEMKEKGIVPNV

SEQ ID NO: 287 - protein, 35 aa, Artificial Sequence
  MODIFIED TYPE crPPR-3
VTYTTLISGLGKAGRLEEALELFEEMKEKGIKPNV

SEQ ID NO: 288 - protein, 35 aa, Artificial Sequence
  MODIFIED TYPE crPPR-4
VTYTTLISAYGKAGRLEKAEELFEEMKEKGIVPNV

SEQ ID NO: 289 - protein, 35 aa, Artificial Sequence
  MODIFIED TYPE crPPR-5
VTYTTLISAYGKAGRLEEALELFEEMKEKGIKPNV

SEQ ID NO: 290 - protein, 35 aa, Artificial Sequence
  MODIFIED TYPE crPPR-6
VTYTTLISAYGKAGRLEKAEELFEEMKEKGIKPNV

SEQ ID NO: 291 - protein, 664 aa, Arabidopsis thaliana
METPLLVGLELRCPPHLFNTHSRPSSSLSIPALSLRILTPTAATTSSAVIELPANVAEAPRSKRHSNSYLARKSAISEVQRSSDFLSSLQRLATVLKVQDLNVILRDFGISGRWQDLIQLFEWMQQHGKISVSTYSSCIKFVGAKNVSKALEIYQSIPDESTKINVYICNSILSCLVKNGKLDSCIKLFDQMKRDGLKPDVVTYNTLLAGCIKVKNGYPKAIELIGELPHNGIQMDSVMYGTVLAICASNGRSEEAENFIQQMKVEGHSPNIYHYSSLLNSYSWKGDYKKADELMTEMKSIGLVPNKVMMTTLLKVYIKGGLFDRSRELLSELESAGYAENEMPYCMLMDGLSKAGKLEEARSIFDDMKGKGVRSDGYANSIMISALCRSKRFKEAKELSRDSETTYEKCDLVMLNTMLCAYCRAGEMESVMRMMKKMDEQAVSPDYNTFHILIKYFIKEKLHLLAYQTTLDMHSKGHRLEEELCSSLIYHLGKIRAQAEAFSVYNMLRYSKRTICKELHEKILHILIQGNLLKDAYIVVKDNAKMISQPTLKKFGRAFMISGNINLVNDVLKVLHGSGHKIDQVQFEIAISRYISQPDKKELLLQLLQWMPGQGYVVDSSTRNLILKNSHMFGRLLIAEILSKHHVASRPMIKSRPEQKFRCK

SEQ ID NO: 292 - protein, 630 aa, Arabidopsis thaliana
MASHLFTRSRISLLKTLKPNPFTSASPIRAISGTPFLSQDPLLATESTDHDPSNHQSTSTPLPPNPATGSPLYQENWRSPIPNTPSFNQSLVPLGFLNQAPAPRIRALSETLDMNSLLNMFADWTASQRWSDMKQLFEVWVRSLDKNGKPNKPDVNLYNHYLRANLMMGASAGDMLDLVAPMEEFSVEPNTASYNLVLKAMYQARETEAAMKLLERMLLLGKDSLPDDESYDLVIGMHFGVGKNDEAMKVMDTALKSGYMLSTSVFTECVRSCVAKGRTDTLVSIIERCKAVDRNKSLCPSWILCNYIAEVAIQEDNSKLAFYAFEFMFKWITRGEMARPSVIFSVDEGLVVAGLASAARTCSSSLVEGSWTILKQSLRGRKAANPASYIAKINAYASLGNLQKAFTSLHELESAYADSEKEVVEEMLSPFTSLYPLVVACSKKGFETLDEVYFQLESLSQGDTPYKSVAALNCIILGCANTWDLDRAYQTFEAISASFGLTPNIDSYNALLYAFGKVKKTFEATNVFEHLVSIGVKPDSRTYSLLVDAHLINRDPKSALTVVDDMIKAGFEPSRETLKKLRRRCVREMDDENDDQVEALAKKFQIRMGSENRRNMLFNIDYSRGRALNN

SEQ ID NO: 293 - protein, 610 aa, Arabidopsis thaliana
MYSLSRILQRSQRYNFAPSSFGAVSKLEVSSGGDKERVFKSFGLIYSKPQGLVRLYSARDVFSRFFGIHKLSSIADAKDKGDEVVREEELSESEEAVPVSGDVPEGVVDDDSLFEPELGSDNDDLEIEEKHSKDGGKPTKKRGQSELYESIVAYKSVKHVLEKWVKEGKDLSQAEVTLAIHNLRKRKSYAMCLQLWEWLGANTQFEFTEANYASQLDLVAKVHSLQKAEIFLKDIPESSRGEVVYRTLLANCVLKHHVNKAEDIFNKMKELKFPTSVFACNQLLLLYSMHDRKKISDVLLLMERENIKPSRATYHFLINSKGLAGDITGMEKIVETIKEEGIELDPELQSILAKYYIRAGLKERAQDLMKEIEGKGLQQTPWVCRSLLPLYADIGDSDNVRRLSRFVDQNPRYDNCISAIKAWGKLKEVEEAEAVFERLVEKYKIFPMMPYFALMEIYTENKMLAKGRDLVKRMGNAGIAIGPSTWHALVKLYIKAGEVGKAELILNRATKDNKMRPMFTTYMAILEEYAKRGDVHNTEKVFMKMKRASYAAQLMQYETVLLAYINAKTPAYGMIERMKADNVFPNKSLAAKLAQVNPFKKCPVSVLLDI

SEQ ID NO: 294 - protein, 590 aa, Arabidopsis thaliana
MFFSFRLLKVLVFHLQIRPCVLLCVSQRKLQNNIINVGVKIQNRFRVVCMGMLAPRKFLQKRRKMEVFKDAADETDQKRWRGLMLEIESTGSAVPVLRQYKTDGDQGLPRDLVLGTLVRFKQLKKWNLVSEILEWLRYQNWWNFSEIDFLMLITAYGKLGNFNGAERVLSVLSKMGSTPNVISYTALMESYGRGGKCNNAEAIFRRMQSSGPEPSAITYQIILKTFVEGDKFKEAEEVFETLLDEKKSPLKPDQKMYHMMIYMYKKAGNYEKARKVFSSMVGKGVPQSTVTYNSLMSFETSYKEVSKIYDQMQRSDIQPDVVSYALLIKAYGRARREEEALSVFEEMLDAGVRPTHKAYNILLDAFAISGMVEQAKTVFKSMRRDRIFPDLWSYTTMLSAYVNASDMEGAEKFFKRIKVDGFEPNIVTYGTLIKGYAKANDVEKMMEVYEKMRLSGIKANQTILTTIMDASGRCKNFGSALGWYKEMESCGVPPDQKAKNVLLSLASTQDELEEAKELTGIRNETATIIARVYGSDDDEEGVEDISSESSDDEDEGDDDDDDARETVLYDKPQEGSLGYGSLQTEELVGL

SEQ ID NO: 295 - protein, 580 aa, Arabidopsis thaliana
MNRISAISTLVTPLPLLPSCSFVPTRRCYPRRATPYSRRINLKPLTSRIVLLTRRRQLGQIVEEVEAAKKRYGRLNTIVMNSVLEACVHCGNIDLALRMFHEMAEPGGIGVDSISYATILKGLGKARRIDEAFQMLETIEYGTAAGTPKLSSSLIYGLLDALINAGDLRRANGLLARYDILLLDHGTPSVLIYNLLMKGYVNSESPQAAINLLDEMLRLRLEPDRLTYNTLIHACIKCGDLDAAMKFFNDMKEKAEEYYDDFLQPDVVTYTTLVKGFGDATDLLSLQEIFLEMKLCENVFIDRTAFTAVVDAMLKCGSTSGALCVFGEILKRSGANEVLRPKPHLYLSMMRAFAVQGDYGMVRNLYLRLWPDSSGSISKAVQQEADNLLMEAALNDGQLDEALGILLSIVRRWKTIPWTTSGGMAAVRLETLLGFSKSILRPHLLSKVIPSEPIESIMIRFEATRPLLGTLQLKNVAMRFFKEQVVPIVDDRGSCIGLLHREDCNNLDAPLVSMMRSPPTCVSTTTSIGRVVDLVLEKKLKMVIVVHCGNFSGSGYSSKAVGAFTRAQLYRLFESEQKLL

SEQ ID NO: 296 - protein, 593 aa, Arabidopsis thaliana
MALLISCGEVTSSQFTVFRLLNQSLDFVSDNVSRLLAPIFTNLRDFEMRLSCIERPPSISGNHSHLCTEKWFSDQKDYDQKEDPEAIFNVLDYILKSSLDRLASLRESVCQTKSFDYDDCLSIHSSIMRDLCLQGKLDAALWLRKKMIYSGVIPGLITHNHLLNGLCKAGYIEKADGLVREMREMGPSPNCVSYNTLIKGLCSVNNVDKALYLFNTMNKYGIRPNRVTCNIIVHALCQKGVIGNNNKKLLEEILDSSQANAPLDIVICTILMDSCFKNGNVVQALEVWKEMSQKNVPADSVVYNVIIRGLCSSGNMVAAYGFMCDMVKRGVNPDVFTYNTLISALCKEGKFDEACDLHGTMQNGGVAPDQISYKVIIQGLCIHGDVNRANEFLLSMLKSSLLPEVLLWNVVIDGYGRYGDTSSALSVLNLMLSYGVKPNVYTNNALIHGYVKGGRLIDAWWVKNEMRSTKIHPDTTTYNLLLGAACTLGHLRLAFQLYDEMLRRGCQPDIITYTELVRGLCWKGRLKKAESLLSRIQATGITIDHVPFLILAKKYTRLQRPGEAYLVYKKWLATRNRGVSCPSILNHMHTEEQ

SEQ ID NO: 297 - protein, 798 aa, Arabidopsis thaliana; identical to SEQ ID NO: 4

SEQ ID NO: 298 - protein, 415 aa, Arabidopsis thaliana
MLSLNLSLKPQHLKLLSCYTDSSAPSIAKKLIKESKLSRDFSQKIQIVDYAPLVQTLSQRRLPDVAHEIFLQTKSVNLLPNYRTLCALMLCFAENGFVLRARTIWDEIINSCFVPDVFVVSKLISAYEQFGCFDEVAKITKDVAARHSKLLPVVSSLAISCFGKNGQLELMEGVIEEMDSKGVLLEAETANVIVRYYSFFGSLDKMEKAYGRVKKFGIVIEEEEIRAVVLAYLKQRKFYRLREFLSDVGLGRRNLGNMLWNSVLLSYAADFKMKSLQREFIGMLDAGFSPDLTTFNIRALAFSRMALFWDLHLTLEHMRRLNIVPDLVTFGCVVDAYMDKRLARNLEFVYNRMNLDDSPLVLTDPLAFEVLGKGDFHLSSEAVLEFSPRKNWTYRKLIGVYLKKKLRRDQIFWNY

SEQ ID NO: 299 - protein, 709 aa, Arabidopsis thaliana
MLLLQQPPLVSTRFHSLYFLTHHHHHHHRFFQPPISAFSATTSASLPSPSPSSSSSYFSSWNGLDTNEEEDNEFSSEVHRRYDFSPLLKFLSRFGPVELALDSESESEASPESLNPVEFDLVESYRAVPAPYWHSLIKSLTSSTSSLGLAYAVVSWLQKHNLCFSYELLYSILIHALGRSEKLYEAFLLSQKQTLTPLTYNALIGACARNNDIEKALNLIAKMRQDGYQSDFVNYSLVIQSLTRSNKIDSVMLLRLYKEIERDKLELDVQLVNDIIMGFAKSGDPSKALQLLGMAQATGLSAKTATLVSIISALADSGRTLEAEALFEELRQSGIKPRTRAYNALLKGYVKTGPLKDAESMVSEMEKRGVSPDEHTYSLLIDAYVNAGRWESARIVLKEMEAGDVQPNSFVFSRLLAGFRDRGEWQKTFQVLKEMKSIGVKPDRQFYNVVIDTFGKFNCLDHAMTTFDRMLSEGIEPDRVTWNTLIDCHCKHGRHIVAEEMFEAMERRGCLPCATTYNIMINSYGDQERWDDMKRLLGKMKSQGILPNVVTHTTLVDVYGKSGRFNDAIECLEEMKSVGLKPSSTMYNALINAYAQRGLSEQAVNAFRVMTSDGLKPSLLALNSLINAFGEDRRDAEAFAVLQYMKENGVKPDVVTYTTLMKALIRVDKFQKVPVVYEEMIMSGCKPDRKARSMLRSALRYMKQTLRAS

SEQ ID NO: 300 - protein, 735 aa, Arabidopsis thaliana
MMIKRSITTNMKALRLIQPHLLKTGSLRTDLLCTISSFFSSCERDFSSISNGNVCFRERLRSGIVDIKKDDAIALFQEMIRSRPLPSLVDFSRFFSAIARTKQFNLVLDFCKQLELNGIAHNIYTLNIMINCFCRCCKTCFAYSVLGKVMKLGYEPDTTTFNTLIKGLFLEGKVSEAVVLVDRMVENGCQPDVVTYNSIVNGICRSGDTSLALDLLRKMEERNVKADVFTYSTIIDSLCRDGCIDAAISLFKEMETKGIKSSVVTYNSLVRGLCKAGKWNDGALLLKDMVSREIVPNVITFNVLLDVFVKEGKLQEANELYKEMITRGISPNIITYNTLMDGYCMQNRLSEANNMLDLMVRNKCSPDIVTFTSLIKGYCMVKRVDDGMKVFRNISKRGLVANAVTYSILVQGFCQSGKIKLAEELFQEMVSHGVLPDVMTYGILLDGLCDNGKLEKALEIFEDLQKSKMDLGIVMYTTIIEGMCKGGKVEDAWNLFCSLPCKGVKPNVMTYTVMISGLCKKGSLSEANILLRKMEEDGNAPNDCTYNTLIRAHLRDGDLTASAKLIEEMKSCGFSADASSIKMVIDMLLSAMKRLTLRYCLSKGSKSRQDLLELSGSEKIRLSSLTFVKMFPCNTITTSLNVNTIEARGMNSAELNRDLRKLRRSSVLKKFKNRDVRVLVTNELLTWGLEDAECDLMVDLELPTDAVHYAHRAGRMRRPGRKMTVVTVCEESQVL

SEQ ID NO: 301 - protein, 1006 aa, Arabidopsis thaliana
MAVTISTNAFVNASLLDESRNSFWRPLFHQPYYNCRRVVRLNSRKLNSKVMFCLNLNTKEVGLQKPGDKGFEFKPSFDQYLQIMESVKTARKKKKFDRLKVEEDDGGGGNGDSVYEVKDMKIKSGELKDETFRKRYSRQEIVSDKRNERVFKRNGEIENHRVATDLKWSKSGESSVALKLSKSGESSVTVPEDESFRKRYSKQEYHRSSDTSRGIERGSRGDELDLVVEERRVQRIAKDARWSKSRESSVAVKWSNSGESSVTMPKDESFRRRYSKQEHHRSSDTSRGIARGSKGDELELVVEERRVQRIAKDVRWSKSDESLVPVSEDESFRRGNPKQEMVRYQRVSDTSRGIERGSKGDGLDLLAEERRIERLANERHEIRSSKLSGTRRIGAKRNDDDDDSLFAMETPAFRFSDESSDIVDKPATSRVEMEDRIEKLAKVLNGADINMPEWQFSKAIRSAKIRYTDYTVMRLIHFLGKLGNWRRVLQVIEWLQRQDRYKSNKIRIIYTTALNVLGKSRRPVEALNVFHAMLLQISSYPDMVAYRSIAVTLGQAGHIKELFYVIDTMRSPPKKKFKPTTLEKWDPRLEPDVVVYNAVLNACVQRKQWEGAFWVLQQLKQRGQKPSPVTYGLIMEVMLACEKYNLVHEFFRKMQKSSIPNALAYRVLVNTLWKEGKSDEAVHTVEDMESRGIVGSAALYYDLARCLCSAGRCNEGLNMVNFVNPVVLKLIENLIYKADLVHTIQFQLKKICRVANKPLVVTYTGLIQACVDSGNIKNAAYIFDQMKKVCSPNLVTCNIMLKAYLQGGLFEEARELFQKMSEDGNHIKNSSDFESRVLPDTYTFNTMLDTCAEQEKWDDFGYAYREMLRHGYHFNAKRHLRMVLEASRAGKEEVMEATWEHMRRSNRIPPSPLIKERFFRKLEKGDHISAISSLADLNGKIEETELRAFSTSAWSRVLSRFEQDSVLRLMDDVNRRLGSRSESSDSVLGNLLSSCKDYLKTRTHNL

SEQ ID NO: 302 - protein, 613 aa, Arabidopsis thaliana
MFVHKLRCYYGFLLKHFENCLLWLVAGNALNCLFIDSSGFQRYLGFGVTNLNGATVKSYKQEGFVIDERGKLKRFNRKKLSRKRCGSLRGRGWKYGSGFVDGIFPVLSPIAQKILSFIQKETDPDKVADVLGALPSTHASWDDLINVSVQLRLNKKWDSIILVCEWILRKSSFQPDVICFNLLIDAYGQKFQYKEAESLYVQLLESRYVPTEDTYALLIKAYCMAGLIERAEVVLVEMQNHHVSPKTIGVTVYNAYIEGLMKRKGNTEEAIDVFQRMKRDRCKPTTETYNLMINLYGKASKSYMSWKLYCEMRSHQCKPNICTYTALVNAFAREGLCEKAEEIFEQLQEDGLEPDVYVYNALMESYSRAGYPYGAAEIFSLMQHMGCEPDRASYNIMVDAYGRAGLHSDAEAVFEEMKRLGIAPTMKSHMLLLSAYSKARDVTKCEAIVKEMSENGVEPDTFVLNSMLNLYGRLGQFTKMEKILAEMENGPCTADISTYNILINIYGKAGFLERIEELFVELKEKNFRPDVVTWTSRIGAYSRKKLYVKCLEVFEEMIDSGCAPDGGTAKVLLSACSSEEQVEQVTSVLRTMHKGVTVSSLVPKLMAKSLTVN

SEQ ID NO: 303 - protein, 822 aa, Arabidopsis thaliana
MATVTNFKLVTPPESSRADKPGATKASDAFQEKKSVSVNYDRGEHEVSVNIGGLRKADIPRRYRIRVENDRFQKDWSVSEVVDRLMALNRWEEVDGVLNSWVGRFARKNFPVLIRELSRRGCIELCVNVFKWMKIQKNYCARNDIYNMMIRLHARHNWVDQARGLFFEMQKWSCKPDAETYDALINAHGRAGQWRWAMNLMDDMLRAAIAPSRSTYNNLINACGSSGNWREALEVCKKMTDNGVGPDLVTHNIVLSAYKSGRQYSKALSYFELMKGAKVRPDTTTFNIIIYCLSKLGQSSQALDLFNSMREKRAECRPDVVTFTSIMHLYSVKGEIENCRAVFEAMVAEGLKPNIVSYNALMGAYAVHGMSGTALSVLGDIKQNGIIPDVVSYTCLLNSYGRSRQPGKAKEVFLMMRKERRKPNVVTYNALIDAYGSNGFLAEAVEIFRQMEQDGIKPNVVSVCTLLAACSRSKKKVNVDTVLSAAQSRGINLNTAAYNSAIGSYINAAELEKAIALYQSMRKKKVKADSVTFTILISGSCRMSKYPEAISYLKEMEDLSIPLTKEVYSSVLCAYSKQGQVTEAESIFNQMKMAGCEPDVIAYTSMLHAYNASEKWGKACELFLEMEANGIEPDSIACSALMRAFNKGGQPSNVFVLMDLMREKEIPFTGAVFFEIFSACNTLQEWKRAIDLIQMMDPYLPSLSIGLTNQMLHLFGKSGKVEAMMKLFYKIIASGVGINLKTYAILLEHLLAVGNWRKYIEVLEWMSGAGIQPSNQMYRDIISFGERSAGIEFEPLIRQKLESLRNKGEGLIPTFRHEGTLL

SEQ ID NO: 304 - protein, 1440 aa, Arabidopsis thaliana
MAVSAGALAFPALSVRATLNPEIKDEQANISSTTSSSQKFTYSRASPAVRWPHLNLREIYDSTPSQTLSSPVSPIAGTPDSGDVVDSIASREEQKTKDETAVATRRRRVKKMNKVALIKAKDWRERVKFLTDKILSLKSNQFVADILDARLVQMTPTDYCFVVKSVGQESWQRALEVFEWLNLRHWHSPNARMVAAILGVLGRWNQESLAVEIFTRAEPTVGDRVQVYNAMMGVYSRSGKFSKAQELVDAMRQRGCVPDLISFNTLINARLKSGGLTPNLAVELLDMVRNSGLRPDAITYNTLLSACSRDSNLDGAVKVFEDMEAHRCQPDLWTYNAMISVYGRCGLAAEAERLFMELELKGFFPDAVTYNSLLYAFARERNTEKVKEVYQQMQKMGFGKDEMTYNTIIHMYGKQGQLDLALQLYKDMKGLSGRNPDAITYTVLIDSLGKANRTVEAAALMSEMLDVGIKPTLQTYSALICGYAKAGKREEAEDTFSCMLRSGTKPDNLAYSVMLDVLLRGNETRKAWGLYRDMISDGHTPSYTLYELMILGLMKENRSDDIQKTIRDMEELCGMNPLEISSVLVKGECFDLAARQLKVAITNGYELENDTLLSILGSYSSSGRHSEAFELLEFLKEHASGSKRLITEALIVLHCKVNNLSAALDEYFADPCVHGWCFGSSTMYETLLHCCVANEHYAEASQVFSDLRLSGCEASESVCKSMVVVYCKLGFPETAHQVVNQAETKGFHFACSPMYTDIIEAYGKQKLWQKAESVVGNLRQSGRTPDLKTWNSLMSAYAQCGCYERARAIFNTMMRDGPSPTVESINILLHALCVDGRLEELYVVVEELQDMGFKISKSSILLMLDAFARAGNIFEVKKIYSSMKAAGYLPTIRLYRMMIELLCKGKRVRDAEIMVSEMEEANFKVELAIWNSMLKMYTAIEDYKKTVQVYQRIKETGLEPDETTYNTLIIMYCRDRRPEEGYLLMQQMRNLGLDPKLDTYKSLISAFGKQKCLEQAEQLFEELLSKGLKLDRSFYHTMMKISRDSGSDSKAEKLLQMMKNAGIEPTLATMHLLMVSYSSSGNPQEAEKVLSNLKDTEVELTTLPYSSVIDAYLRSKDYNSGIERLLEMKKEGLEPDHRIWTCFVRAASFSKEKIEVMLLLKALEDIGFDLPIRLLAGRPELLVSEVDGWFEKLKSIEDNAALNFVNALLNLLWAFELRATASWVFQLGIKRGIFSLDVFRVADKDWGADFRRLSGGAALVALTLWLDHMQDASLEGYPESPKSVVLITGTAEYNGISLDKTLKACLWEMGSPFLPCKTRTGLLVAKAHSLRMWLKDSPFCFDLELKDSVSLPESNSMDLIDGCFIRRGLVPAFNHIKERLGGFVSPKKFSRLALLPDEMRERVIKTDIEGHRQKLEKMKKKKMGNETNGINTRRKFVRSK

SEQ ID NO: 305 - protein, 499 aa, Arabidopsis thaliana
MALIQNPVQGTTSAYANEIAQLGFSRSVVQQHISSPVYFRCIPTISITPTMCSTKVPNERTEKMNSGLISTRHQVDPKKELSRILRTDAAVKGIERKANSEKYLTLWPKAVLEALDEAIKENRWQSALKIFNLLRKQHWYEPRCKTYTKLFKVLGNCKQPDQASLLFEVMLSEGLKPTIDVYTSLISVYGKSELLDKAFSTLEYMKSVSDCKPDVFTFTVLISCCCKLGRFDLVKSIVLEMSYLGVGCSTVTYNTIIDGYGKAGMFEEMESVLADMIEDGDSLPDVCTLNSIIGSYGNGRNMRKMESWYSRFQLMGVQPDITTFNILILSFGKAGMYKKMCSVMDFMEKRFFSLTTVTYNIVIETFGKAGRIEKMDDVFRKMKYQGVKPNSITYCSLVNAYSKAGLVVKIDSVLRQIVNSDVVLDTPFFNCIINAYGQAGDLATMKELYIQMEERKCKPDKITFATMIKTYTAHGIFDAVQELEKQMISSDIGKKRLTE

SEQ ID NO: 306 - protein, 551 aa, Arabidopsis thaliana
MVLIHTSVGFFKRFSTSATPSTSSASDWKTQQTLFRVATEISSILLQRRNWITHLQYVKSKLPRSTLTSPVFLQILRETRKCPKTTLDFFDFAKTHLRFEPDLKSHCRVIEVAAESGLLERAEMLLRPLVETNSVSLVVGEMHRWFEGEVSLSVSLSLVLEYYALKGSHHNGLEVFGFMRRLRLSPSQSAYNSLLGSLVKENQFRVALCLYSAMVRNGIVSDELTWDLIAQILCEQGRSKSVFKLMETGVESCKIYTNLVECYSRNGEFDAVFSLIHEMDDKKLELSFCSYGCVLDDACRLGDAEFIDKVLCLMVEKKFVTLGDSAVNDKIIERLCDMGKTFASEMLFRKACNGETVRLWDSTYGCMLKALSRKKRTKEAVDVYRMICRKGITVLDESCYIEFANALCRDDNSSEEEEELLVDVIKRGKEDGNPQRSFLIRLWKWRSGKLEKALVLHEKIKKMKGSLDVNAYNAVLDRLMMRQKEMVEEAVVVFEYMKEINSVNSKSFTIMIQGLCRVKEMKKAMRSHDEMLRLGLKPDLVTYKRLILGFK

SEQ ID NO: 307 - protein, 508 aa, Arabidopsis thaliana
MVSLSTSTSHAPPLPTNRRTAERTFTVRCISISPREPNYAITSDKSNNTSLSLRETRQSKWLINAEDVNERDSKEIKEDKNTKIASRKAISIILRREATKSIIEKKKGSKKLLPRTVLESLHERITALRWESAIQVFELLREQLWYKPNVGIYVKLIVMLGKCKQPEKAHELFQEMINEGCVVNHEVYTALVSAYSRSGRFDAAFTLLERMKSSHNCQPDVHTYSILIKSFLQVFAFDKVQDLLSDMRRQGIRPNTITYNTLIDAYGKAKMFVEMESTLIQMLGEDDCKPDSWTMNSTLRAFGGNGQIEMMENCYEKFQSSGIEPNIRTFNILLDSYGKSGNYKKMSAVMEYMQKYHYSWTIVTYNVVIDAFGRAGDLKQMEYLFRLMQSERIFPSCVTLCSLVRAYGRASKADKIGGVLRFIENSDIRLDLVFFNCLVDAYGRMEKFAEMKGVLELMEKKGFKPDKITYRTMVKAYRISGMTTHVKELHGVVESVGEAQVVVKKPDF

SEQ ID NO: 308 - protein, 723 aa, Arabidopsis thaliana
MSMASSSLATQSFFSSFPLSHRLHFPVPYLLLRSSFFRKPLSLSATSPSSSSSSPSIFLSCFDDALPDKIQQPENSTINSEESECEEEDDEEGDDFTDPILKFFKSRTLTSESTADPARESKFSLQKNRRTSWHLAPDFADPETEIESKPEESVFVTNQQTLGVHIPFESGVAREILELAKNLKENQTLGEMLSGFERRVSDTECVEALVMMGESGFVKSCLYFYEWMSLQEPSLASPRACSVLFTLLGRERMADYILLLLSNLPDKEEFRDVRLYNAAISGLSASQRYDDAWEVYEAMDKINVYPDNVTCAILITTLRKAGRSAKEVWEIFEKMSEKGVKWSQDVFGGLVKSFCDEGLKEEALVIQTEMEKKGIRSNTIVYNTLMDAYNKSNHIEEVEGLFTEMRDKGLKPSAATYNILMDAYARRMQPDIVETLLREMEDLGLEPNVKSYTCLISAYGRTKKMSDMAADAFLRMKKVGLKPSSHSYTALIHAYSVSGWHEKAYASFEEMCKEGIKPSVETYTSVLDAFRRSGDTGKLMEIWKLMLREKIKGTRITYNTLLDGFAKQGLYIEARDVVSEFSKMGLQPSVMTYNMLMNAYARGGQDAKLPQLLKEMAALNLKPDSITYSTMIYAFVRVRDFKRAFFYHKMMVKSGQVPDPRSYEKLRAILEDKAKTKNRKDKTAILGIINSKFGRVKAKTKGKKDEFWKYKTNRTTSPGRHRS

SEQ ID NO: 309 - protein, 4 aa, Artificial Sequence
  crPPR N terminal side
MGNS

SEQ ID NO: 310 - protein, 14 aa, Artificial Sequence
  crPPR C terminal side
VTYTTLISGLGKAG

SEQ ID NO: 311 - protein, 35 aa, Artificial Sequence
  crPPR(7L/31F)
VTYTTLLSGLGKAGRLEEALELFEEMKEKGFVPNV

SEQ ID NO: 312 - protein, 14 aa, Artificial Sequence
  crPPR(7L/31F)
VTYTTLLSGLGKAG

SEQ ID NO: 313 - protein, 35 aa, Artificial Sequence
  MODIFIED TYPE crPPR(7L/31F)
VTYTTLISGLGKAGRLEEALELFEEMKEKGFVPNV

SEQ ID NO: 314 - protein, 35 aa, Artificial Sequence
  MODIFIED TYPE crPPR(7L/31F)
VTYTTLLSALGKAGRLEEALELFEEMKEKGFVPNV

SEQ ID NO: 315 - protein, 14 aa, Artificial Sequence
  MODIFIED TYPE crPPR(7L/31F)
VTYTTLLSALGKAG

SEQ ID NO: 316 - protein, 35 aa, Artificial Sequence
  MODIFIED TYPE crPPR(7L/31F)
VTYTTLLSGYGKAGRLEEALELFEEMKEKGFVPNV

SEQ ID NO: 317 - protein, 14 aa, Artificial Sequence
  MODIFIED TYPE crPPR(7L/31F)
VTYTTLLSGYGKAG

SEQ ID NO: 318 - protein, 35 aa, Artificial Sequence
  MODIFIED TYPE crPPR(7L/31F)
VTYTTLLSGLGKAGRLEKALELFEEMKEKGFVPNV

SEQ ID NO: 319 - protein, 35 aa, Artificial Sequence
  MODIFIED TYPE crPPR(7L/31F)
VTYTTLLSGLGKAGRLEEAEELFEEMKEKGFVPNV

SEQ ID NO: 320 - protein, 35 aa, Artificial Sequence
  MODIFIED TYPE crPPR(7L/31F)
VTYTTLLSGLGKAGRLEEALELFEEMKEEGFVPNV

SEQ ID NO: 321 - protein, 35 aa, Artificial Sequence
  MODIFIED TYPE crPPR(7L/31F)
VTYTTLLSGLGKAGRLEEALELFEEMKEKGIVPNV

SEQ ID NO: 322 - protein, 35 aa, Artificial Sequence
  MODIFIED TYPE crPPR(7L/31F)
VTYTTLLSGLGKAGRLEEALELFEEMKEKGFKPNV

SEQ ID NO: 323 - protein, 35 aa, Artificial Sequence
  MODIFIED TYPE crPPR(7L/31F)
VTYTTLLSAYGKAGRLEEALELFEEMKEKGFVPNV

SEQ ID NO: 324 - protein, 14 aa, Artificial Sequence
  MODIFIED TYPE crPPR(7L/31F)
VTYTTLLSAYGKAG

SEQ ID NO: 325 - protein, 35 aa, Artificial Sequence
  MODIFIED TYPE crPPR(7L/31F)
VTYTTLLSGLGKAGRLEHALELFEEMKEKGFVPNV

SEQ ID NO: 326 - protein, 35 aa, Artificial Sequence
  MODIFIED TYPE crPPR(7L/31F)
VTYTTLLSGLGKAGRLERALELFEEMKEKGFVPNV

SEQ ID NO: 327 - protein, 35 aa, Artificial Sequence
  MODIFIED TYPE crPPR(7L/31F)
VTYTTLLSGLGKAGRLEKALELFEEMKEKGVVPNV

SEQ ID NO: 328 - protein, 35 aa, Artificial Sequence
  MODIFIED TYPE crPPR(7L/31F)
VTYTTLLSGLGKAGRLEKALELFEEMKEKGLVPNV

SEQ ID NO: 329 - protein, 35 aa, Artificial Sequence
  MODIFIED TYPE crPPR(7L/31F)
VTYTTLLSGLGKAGRLEEALELFEEMKEKGFHPNV

SEQ ID NO: 330 - protein, 35 aa, Artificial Sequence
  MODIFIED TYPE crPPR(7L/31F)
VTYTTLLSGLGKAGRLEEALELFEEMKEKGFRPNV

SEQ ID NO: 331 - protein, 35 aa, Artificial Sequence
  MODIFIED TYPE crPPR(7L/31F)
VTYTTLLSAFGKAGRLEEALELFEEMKEKGFVPNV

SEQ ID NO: 332 - protein, 14 aa, Artificial Sequence
  MODIFIED TYPE crPPR(7L/31F)
VTYTTLLSAFGKAG

SEQ ID NO: 333 - protein, 35 aa, Artificial Sequence
  MODIFIED TYPE crPPR(7L/31F)
VTYTTLLSAWGKAGRLEEALELFEEMKEKGFVPNV

SEQ ID NO: 334 - protein, 14 aa, Artificial Sequence
  MODIFIED TYPE crPPR(7L/31F)
VTYTTLLSAWGKAG

SEQ ID NO: 335 - protein, 298 aa, Artificial Sequence
  crPPR
MGNSVTYTTLISGLGKAGRLEEALELFEEMKEKGIVPNVVTYTTLISGLGKAGRLEEALELFEEMKEKGIVPNVVTYTTLISGLGKAGRLEEALELFEEMKEKGIVPNVVTYTTLISGLGKAGRLEEALELFEEMKEKGIVPNVVTYTTLISGLGKAGRLEEALELFEEMKEKGIVPNVVTYTTLISGLGKAGRLEEALELFEEMKEKGIVPNVVTYTTLISGLGKAGRLEEALELFEEMKEKGIVPNVVTYTTLISGLGKAGRLEEALELFEEMKEKGIVPNVVTYTTLISGLGKAG

SEQ ID NO: 336 - protein, 298 aa, Artificial Sequence
  MODIFIED TYPE crPPR(7L/31F)
MGNSVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAG

SEQ ID NO: 337 - protein, 298 aa, Artificial Sequence
  MODIFIED TYPE crPPR(7L/31F)
MGNSVTYTTLISGLGKAGRLEEALELFEEMKEKGFVPNVVTYTTLISGLGKAGRLEEALELFEEMKEKGFVPNVVTYTTLISGLGKAGRLEEALELFEEMKEKGFVPNVVTYTTLISGLGKAGRLEEALELFEEMKEKGFVPNVVTYTTLISGLGKAGRLEEALELFEEMKEKGFVPNVVTYTTLISGLGKAGRLEEALELFEEMKEKGFVPNVVTYTTLISGLGKAGRLEEALELFEEMKEKGFVPNVVTYTTLISGLGKAGRLEEALELFEEMKEKGFVPNVVTYTTLISGLGKAG

SEQ ID NO: 338 - protein, 298 aa, Artificial Sequence
  MODIFIED TYPE crPPR(7L/31F)
MGNSVTYTTLLSALGKAGRLEEALELFEEMKEKGFVPNVVTYTTLLSALGKAGRLEEALELFEEMKEKGFVPNVVTYTTLLSALGKAGRLEEALELFEEMKEKGFVPNVVTYTTLLSALGKAGRLEEALELFEEMKEKGFVPNVVTYTTLLSALGKAGRLEEALELFEEMKEKGFVPNVVTYTTLLSALGKAGRLEEALELFEEMKEKGFVPNVVTYTTLLSALGKAGRLEEALELFEEMKEKGFVPNVVTYTTLLSALGKAGRLEEALELFEEMKEKGFVPNVVTYTTLLSALGKAG

SEQ ID NO: 339 - protein, 298 aa, Artificial Sequence
  MODIFIED TYPE crPPR(7L/31F)
MGNSVTYTTLLSGYGKAGRLEEALELFEEMKEKGFVPNVVTYTTLLSGYGKAGRLEEALELFEEMKEKGFVPNVVTYTTLLSGYGKAGRLEEALELFEEMKEKGFVPNVVTYTTLLSGYGKAGRLEEALELFEEMKEKGFVPNVVTYTTLLSGYGKAGRLEEALELFEEMKEKGFVPNVVTYTTLLSGYGKAGRLEEALELFEEMKEKGFVPNVVTYTTLLSGYGKAGRLEEALELFEEMKEKGFVPNVVTYTTLLSGYGKAGRLEEALELFEEMKEKGFVPNVVTYTTLLSGYGKAG

SEQ ID NO: 340 - protein, 298 aa, Artificial Sequence
  MODIFIED TYPE crPPR(7L/31F)
MGNSVTYTTLLSGLGKAGRLEKALELFEEMKEKGFVPNVVTYTTLLSGLGKAGRLEKALELFEEMKEKGFVPNVVTYTTLLSGLGKAGRLEKALELFEEMKEKGFVPNVVTYTTLLSGLGKAGRLEKALELFEEMKEKGFVPNVVTYTTLLSGLGKAGRLEKALELFEEMKEKGFVPNVVTYTTLLSGLGKAGRLEKALELFEEMKEKGFVPNVVTYTTLLSGLGKAGRLEKALELFEEMKEKGFVPNVVTYTTLLSGLGKAGRLEKALELFEEMKEKGFVPNVVTYTTLLSGLGKAG

SEQ ID NO: 341 - protein, 298 aa, Artificial Sequence
  MODIFIED TYPE crPPR(7L/31F)
MGNSVTYTTLLSGLGKAGRLEEAEELFEEMKEKGFVPNVVTYTTLLSGLGKAGRLEEAEELFEEMKEKGFVPNVVTYTTLLSGLGKAGRLEEAEELFEEMKEKGFVPNVVTYTTLLSGLGKAGRLEEAEELFEEMKEKGFVPNVVTYTTLLSGLGKAGRLEEAEELFEEMKEKGFVPNVVTYTTLLSGLGKAGRLEEAEELFEEMKEKGFVPNVVTYTTLLSGLGKAGRLEEAEELFEEMKEKGFVPNVVTYTTLLSGLGKAGRLEEAEELFEEMKEKGFVPNVVTYTTLLSGLGKAG

SEQ ID NO: 342 - protein, 298 aa, Artificial Sequence
  MODIFIED TYPE crPPR(7L/31F)
MGNSVTYTTLLSGLGKAGRLEEALELFEEMKEEGFVPNVVTYTTLLSGLGKAGRLEEALELFEEMKEEGFVPNVVTYTTLLSGLGKAGRLEEALELFEEMKEEGFVPNVVTYTTLLSGLGKAGRLEEALELFEEMKEEGFVPNVVTYTTLLSGLGKAGRLEEALELFEEMKEEGFVPNVVTYTTLLSGLGKAGRLEEALELFEEMKEEGFVPNVVTYTTLLSGLGKAGRLEEALELFEEMKEEGFVPNVVTYTTLLSGLGKAGRLEEALELFEEMKEEGFVPNVVTYTTLLSGLGKAG

SEQ ID NO: 343 - protein, 298 aa, Artificial Sequence
  MODIFIED TYPE crPPR(7L/31F)
MGNSVTYTTLLSGLGKAGRLEEALELFEEMKEKGIVPNVVTYTTLLSGLGKAGRLEEALELFEEMKEKGIVPNVVTYTTLLSGLGKAGRLEEALELFEEMKEKGIVPNVVTYTTLLSGLGKAGRLEEALELFEEMKEKGIVPNVVTYTTLLSGLGKAGRLEEALELFEEMKEKGIVPNVVTYTTLLSGLGKAGRLEEALELFEEMKEKGIVPNVVTYTTLLSGLGKAGRLEEALELFEEMKEKGIVPNVVTYTTLLSGLGKAGRLEEALELFEEMKEKGIVPNVVTYTTLLSGLGKAG

SEQ ID NO: 344 - protein, 298 aa, Artificial Sequence
  MODIFIED TYPE crPPR(7L/31F)
MGNSVTYTTLLSGLGKAGRLEEALELFEEMKEKGFKPNVVTYTTLLSGLGKAGRLEEALELFEEMKEKGFKPNVVTYTTLLSGLGKAGRLEEALELFEEMKEKGFKPNVVTYTTLLSGLGKAGRLEEALELFEEMKEKGFKPNVVTYTTLLSGLGKAGRLEEALELFEEMKEKGFKPNVVTYTTLLSGLGKAGRLEEALELFEEMKEKGFKPNVVTYTTLLSGLGKAGRLEEALELFEEMKEKGFKPNVVTYTTLLSGLGKAGRLEEALELFEEMKEKGFKPNVVTYTTLLSGLGKAG

SEQ ID NO: 345 - protein, 298 aa, Artificial Sequence
  MODIFIED TYPE crPPR(7L/31F)
MGNSVTYTTLLSAYGKAGRLEEALELFEEMKEKGFVPNVVTYTTLLSAYGKAGRLEEALELFEEMKEKGFVPNVVTYTTLLSAYGKAGRLEEALELFEEMKEKGFVPNVVTYTTLLSAYGKAGRLEEALELFEEMKEKGFVPNVVTYTTLLSAYGKAGRLEEALELFEEMKEKGFVPNVVTYTTLLSAYGKAGRLEEALELFEEMKEKGFVPNVVTYTTLLSAYGKAGRLEEALELFEEMKEKGFVPNVVTYTTLLSAYGKAGRLEEALELFEEMKEKGFVPNVVTYTTLLSAYGKAG

SEQ ID NO: 346 - protein, 298 aa, Artificial Sequence
  MODIFIED TYPE crPPR(7L/31F)
MGNSVTYTTLLSGLGKAGRLEHALELFEEMKEKGFVPNVVTYTTLLSGLGKAGRLEHALELFEEMKEKGFVPNVVTYTTLLSGLGKAGRLEHALELFEEMKEKGFVPNVVTYTTLLSGLGKAGRLEHALELFEEMKEKGFVPNVVTYTTLLSGLGKAGRLEHALELFEEMKEKGFVPNVVTYTTLLSGLGKAGRLEHALELFEEMKEKGFVPNVVTYTTLLSGLGKAGRLEHALELFEEMKEKGFVPNVVTYTTLLSGLGKAGRLEHALELFEEMKEKGFVPNVVTYTTLLSGLGKAG

SEQ ID NO: 347 - protein, 298 aa, Artificial Sequence
  MODIFIED TYPE crPPR(7L/31F)
MGNSVTYTTLLSGLGKAGRLERALELFEEMKEKGFVPNVVTYTTLLSGLGKAGRLERALELFEEMKEKGFVPNVVTYTTLLSGLGKAGRLERALELFEEMKEKGFVPNVVTYTTLLSGLGKAGRLERALELFEEMKEKGFVPNVVTYTTLLSGLGKAGRLERALELFEEMKEKGFVPNVVTYTTLLSGLGKAGRLERALELFEEMKEKGFVPNVVTYTTLLSGLGKAGRLERALELFEEMKEKGFVPNVVTYTTLLSGLGKAGRLERALELFEEMKEKGFVPNVVTYTTLLSGLGKAG

SEQ ID NO: 348 - protein, 298 aa, Artificial Sequence
  MODIFIED TYPE crPPR(7L/31F)
MGNSVTYTTLLSGLGKAGRLEKALELFEEMKEKGVVPNVVTYTTLLSGLGKAGRLEKALELFEEMKEKGVVPNVVTYTTLLSGLGKAGRLEKALELFEEMKEKGVVPNVVTYTTLLSGLGKAGRLEKALELFEEMKEKGVVPNVVTYTTLLSGLGKAGRLEKALELFEEMKEKGVVPNVVTYTTLLSGLGKAGRLEKALELFEEMKEKGVVPNVVTYTTLLSGLGKAGRLEKALELFEEMKEKGVVPNVVTYTTLLSGLGKAGRLEKALELFEEMKEKGVVPNVVTYTTLLSGLGKAG

SEQ ID NO: 349 - protein, 298 aa, Artificial Sequence
  MODIFIED TYPE crPPR(7L/31F)
MGNSVTYTTLLSGLGKAGRLEKALELFEEMKEKGLVPNVVTYTTLLSGLGKAGRLEKALELFEEMKEKGLVPNVVTYTTLLSGLGKAGRLEKALELFEEMKEKGLVPNVVTYTTLLSGLGKAGRLEKALELFEEMKEKGLVPNVVTYTTLLSGLGKAGRLEKALELFEEMKEKGLVPNVVTYTTLLSGLGKAGRLEKALELFEEMKEKGLVPNVVTYTTLLSGLGKAGRLEKALELFEEMKEKGLVPNVVTYTTLLSGLGKAGRLEKALELFEEMKEKGLVPNVVTYTTLLSGLGKAG

SEQ ID NO: 350 - protein, 298 aa, Artificial Sequence
  MODIFIED TYPE crPPR(7L/31F)
MGNSVTYTTLLSGLGKAGRLEEALELFEEMKEKGFHPNVVTYTTLLSGLGKAGRLEEALELFEEMKEKGFHPNVVTYTTLLSGLGKAGRLEEALELFEEMKEKGFHPNVVTYTTLLSGLGKAGRLEEALELFEEMKEKGFHPNVVTYTTLLSGLGKAGRLEEALELFEEMKEKGFHPNVVTYTTLLSGLGKAGRLEEALELFEEMKEKGFHPNVVTYTTLLSGLGKAGRLEEALELFEEMKEKGFHPNVVTYTTLLSGLGKAGRLEEALELFEEMKEKGFHPNVVTYTTLLSGLGKAG

SEQ ID NO: 351 - protein, 298 aa, Artificial Sequence
  MODIFIED TYPE crPPR(7L/31F)
MGNSVTYTTLLSGLGKAGRLEEALELFEEMKEKGFRPNVVTYTTLLSGLGKAGRLEEALELFEEMKEKGFRPNVVTYTTLLSGLGKAGRLEEALELFEEMKEKGFRPNVVTYTTLLSGLGKAGRLEEALELFEEMKEKGFRPNVVTYTTLLSGLGKAGRLEEALELFEEMKEKGFRPNVVTYTTLLSGLGKAGRLEEALELFEEMKEKGFRPNVVTYTTLLSGLGKAGRLEEALELFEEMKEKGFRPNVVTYTTLLSGLGKAGRLEEALELFEEMKEKGFRPNVVTYTTLLSGLGKAG

SEQ ID NO: 352 - protein, 298 aa, Artificial Sequence
  MODIFIED TYPE crPPR(7L/31F)
MGNSVTYTTLLSAFGKAGRLEEALELFEEMKEKGFVPNVVTYTTLLSAFGKAGRLEEALELFEEMKEKGFVPNVVTYTTLLSAFGKAGRLEEALELFEEMKEKGFVPNVVTYTTLLSAFGKAGRLEEALELFEEMKEKGFVPNVVTYTTLLSAFGKAGRLEEALELFEEMKEKGFVPNVVTYTTLLSAFGKAGRLEEALELFEEMKEKGFVPNVVTYTTLLSAFGKAGRLEEALELFEEMKEKGFVPNVVTYTTLLSAFGKAGRLEEALELFEEMKEKGFVPNVVTYTTLLSAFGKAG

SEQ ID NO: 353 - protein, 298 aa, Artificial Sequence
  MODIFIED TYPE crPPR(7L/31F)
MGNSVTYTTLLSAWGKAGRLEEALELFEEMKEKGFVPNVVTYTTLLSAWGKAGRLEEALELFEEMKEKGFVPNVVTYTTLLSAWGKAGRLEEALELFEEMKEKGFVPNVVTYTTLLSAWGKAGRLEEALELFEEMKEKGFVPNVVTYTTLLSAWGKAGRLEEALELFEEMKEKGFVPNVVTYTTLLSAWGKAGRLEEALELFEEMKEKGFVPNVVTYTTLLSAWGKAGRLEEALELFEEMKEKGFVPNVVTYTTLLSAWGKAGRLEEALELFEEMKEKGFVPNVVTYTTLLSAWGKAG

SEQ ID NO: 354 - protein, 298 aa, Artificial Sequence
  MODIFIED TYPE crPPR(7L/31F)
MGNSVTYTTLLSGLGKAGRLEKALELFEEMKEKGFVPNVVTYTTLLSGLGKAGRLEKALELFEEMKEKGFVPNVVTYTTLLSGLGKAGRLEKALELFEEMKEKGFVPNVVTYTTLLSGLGKAGRLEKALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAG

SEQ ID NO: 355 - protein, 298 aa, Artificial Sequence
  MODIFIED TYPE crPPR(7L/31F)
MGNSVTYTTLLSGLGKAGRLEKALELFEEMKEKGFVPNVVTYTTLLSGLGKAGRLEKALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAG

SEQ ID NO: 356 - protein, 298 aa, Artificial Sequence
  MODIFIED TYPE crPPR(7L/31F)
MGNSVTYTTLLSGLGKAGRLEEALELFEEMKEKGIVPNVVTYTTLLSGLGKAGRLEEALELFEEMKEKGIVPNVVTYTTLLSGLGKAGRLEEALELFEEMKEKGIVPNVVTYTTLLSGLGKAGRLEEALELFEEMKEKGIVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAG

SEQ ID NO: 357 - protein, 298 aa, Artificial Sequence
  MODIFIED TYPE crPPR(7L/31F)
MGNSVTYTTLLSGLGKAGRLEEALELFEEMKEKGIVPNVVTYTTLLSGLGKAGRLEEALELFEEMKEKGIVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAG

SEQ ID NO: 358 - protein, 298 aa, Artificial Sequence
  MODIFIED TYPE crPPR(7L/31F)
MGNSVTYTTLLSGLGKAGRLEEALELFEEMKEKGFKPNVVTYTTLLSGLGKAGRLEEALELFEEMKEKGFKPNVVTYTTLLSGLGKAGRLEEALELFEEMKEKGFKPNVVTYTTLLSGLGKAGRLEEALELFEEMKEKGFKPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAG

SEQ ID NO: 359 - protein, 298 aa, Artificial Sequence
  MODIFIED TYPE crPPR(7L/31F)
MGNSVTYTTLLSGLGKAGRLEEALELFEEMKEKGFKPNVVTYTTLLSGLGKAGRLEEALELFEEMKEKGFKPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAG

SEQ ID NO: 360 - protein, 298 aa, Artificial Sequence
  MODIFIED TYPE crPPR(7L/31F)
MGNSVTYTTLLSAYGKAGRLEEALELFEEMKEKGFVPNVVTYTTLLSAYGKAGRLEEALELFEEMKEKGFVPNVVTYTTLLSAYGKAGRLEEALELFEEMKEKGFVPNVVTYTTLLSAYGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAG

SEQ ID NO: 361 - protein, 298 aa, Artificial Sequence
  MODIFIED TYPE crPPR(7L/31F)
MGNSVTYTTLLSAYGKAGRLEEALELFEEMKEKGFVPNVVTYTTLLSAYGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAGRLEEALELFEEMKEKGFVPNVVTYTTLLSGLGKAG

SEQ ID NO: 362 - protein, 58 aa, Arabidopsis thaliana
AGTKSDQEEDDLEDGFSELEGSKSGQGSTSSDEDEGKLSADEEEEEELDLIETDVSRK

SEQ ID NO: 363 - protein, 35 aa, Arabidopsis thaliana
VLYRTLLANCVAAGNVKKSELVFNKMKDLGFPLSG

SEQ ID NO: 364 - protein, 34 aa, Arabidopsis thaliana
FTCDQMLLLHKRIDRKKIADVLLLMEKENIKPSL

SEQ ID NO: 365 - protein, 35 aa, Arabidopsis thaliana
LTYKILIDVKGATNDISGMEQILETMKDEGVELDF

SEQ ID NO: 366 - protein, 35 aa, Arabidopsis thaliana
QTQALTARHYSGAGLKDKAEKVLKEMEGESLEANR

SEQ ID NO: 367 - protein, 32 aa, Arabidopsis thaliana
RAFKDLLSIYASLGREDEVKRIWKICESKPYF

SEQ ID NO: 368 - protein, 35 aa, Arabidopsis thaliana
EESLAAIQAFGKLNKVQEAEAIFEKIVKMDRRASS

SEQ ID NO: 369 - protein, 35 aa, Arabidopsis thaliana
STYSVLLRVYVDHKMLSKGKDLVKRMAESGCRIEA

SEQ ID NO: 370 - protein, 36 aa, Arabidopsis thaliana
TTWDALIKLYVEAGEVEKADSLLDKASKQSHTKLMM

SEQ ID NO: 371 - protein, 35 aa, Arabidopsis thaliana
NSFMYIMDEYSKRGDVHNTEKIFLKMREAGYTSRL

SEQ ID NO: 372 - protein, 55 aa, Arabidopsis thaliana
RQFQALMQAYINAKSPAYGMRDRLKADNIFPNKSMAAQLAQGDPFKKTAISDILD

SEQ ID NO: 373 - protein, 35 aa, Artificial Sequence
  MODIFIED TYPE P63
VLYRTLLANCVAAGNVKKSELVFNKMKDLGIKLSG

SEQ ID NO: 374 - protein, 35 aa, Artificial Sequence
  MODIFIED TYPE P63
LTYKILIDVKGATNDISGMEQILETMKDEGIKLDF

SEQ ID NO: 375 - protein, 35 aa, Artificial Sequence
  MODIFIED TYPE P63
QTQALTARHYSGAGLKDKAEKVLKEMEGESIKANR

SEQ ID NO: 376 - protein, 32 aa, Artificial Sequence
  MODIFIED TYPE P63
RAFKDLLSIYASLGREDEVKRIWKICEIKPYF

SEQ ID NO: 377 - protein, 35 aa, Artificial Sequence
  MODIFIED TYPE P63
EESLAAIQAFGKLNKVQEAEAIFEKIVKMDIKASS

SEQ ID NO: 378 - protein, 35 aa, Artificial Sequence
  MODIFIED TYPE P63
STYSVLLRVYVDHKMLSKGKDLVKRMAESGIKIEA

SEQ ID NO: 379 - protein, 36 aa, Artificial Sequence
  MODIFIED TYPE P63
TTWDALIKLYVEAGEVEKADSLLDKASKQSHIKLMM

SEQ ID NO: 380 - protein, 35 aa, Artificial Sequence
  MODIFIED TYPE P63
NSFMYIMDEYSKRGDVHNTEKIFLKMREAGIKSRL

SEQ ID NO: 381 - protein, 35 aa, Artificial Sequence
  MODIFIED TYPE P63
VLYRTLLAAYVAAGNVKKSELVFNKMKDLGIPLSG

SEQ ID NO: 382 - protein, 34 aa, Artificial Sequence
  MODIFIED TYPE P63
FTCDQMLLAYKRIDRKKKADVLLLMEKENIKPSL

SEQ ID NO: 383 - protein, 35 aa, Artificial Sequence
  MODIFIED TYPE P63
LTYKILIDAYGATNDISKMEQILETMKDEGIELDF

SEQ ID NO: 384 - protein, 35 aa, Artificial Sequence
  MODIFIED TYPE P63
QTQALTARAYSGAGLKDKAEKVLKEMEGESIEANR

SEQ ID NO: 385 - protein, 32 aa, Artificial Sequence
  MODIFIED TYPE P63
RAFKDLLSAYASLGREDKVKRIWKICEIKPYF

SEQ ID NO: 386 - protein, 35 aa, Artificial Sequence
  MODIFIED TYPE P63
EESLAAIQAYGKLNKVQKAEAIFEKIVKMDIRASS

SEQ ID NO: 387 - protein, 35 aa, Artificial Sequence
  MODIFIED TYPE P63
STYSVLLRAYVDHKMLSKGKDLVKRMAESGIRIEA

SEQ ID NO: 388 - protein, 36 aa, Artificial Sequence
  MODIFIED TYPE P63
TTWDALIKAYVEAGEVEKADSLLDKASKQSHIKLMM

SEQ ID NO: 389 - protein, 35 aa, Artificial Sequence
  MODIFIED TYPE P63
NSFMYIMDAYSKRGDVHKTEKIFLKMREAGITSRL

SEQ ID NO: 390 - protein, 58 aa, Arabidopsis thaliana
SAHLSQTTPNFSPLQTPKSDFSGRQSTRFVSPATNNHRQTRQNPNYNHRPYGASSSPR

SEQ ID NO: 391 - protein, 35 aa, Arabidopsis thaliana
KLASAMISTLGRYGKVTIAKRIFETAFAGGYGNTV

SEQ ID NO: 392 - protein, 35 aa, Arabidopsis thaliana
YAFSALISAYGRSGLHEEAISVFNSMKEYGLRPNL

SEQ ID NO: 393 - protein, 36 aa, Arabidopsis thaliana
VTYNAVIDACGKGGMEFKQVAKFFDEMQRNGVQPDR

SEQ ID NO: 394 - protein, 35 aa, Arabidopsis thaliana
ITFNSLLAVCSRGGLWEAARNLFDEMTNRRIEQDV

SEQ ID NO: 395 - protein, 35 aa, Arabidopsis thaliana
FSYNTLLDAICKGGQMDLAFEILAQMPVKRIMPNV

SEQ ID NO: 396 - protein, 35 aa, Arabidopsis thaliana
VSYSTVIDGFAKAGRFDEALNLFGEMRYLGIALDR

SEQ ID NO: 397 - protein, 35 aa, Arabidopsis thaliana
VSYNTLLSIYTKVGRSEEALDILREMASVGIKKDV

SEQ ID NO: 398 - protein, 35 aa, Arabidopsis thaliana
VTYNALLGGYGKQGKYDEVKKVFTEMKREHVLPNL

SEQ ID NO: 399 - protein, 35 aa, Arabidopsis thaliana
LTYSTLIDGYSKGGLYKEAMEIFREFKSAGLRADV

SEQ ID NO: 400 - protein, 35 aa, Arabidopsis thaliana
VLYSALIDALCKNGLVGSAVSLIDEMTKEGISPNV

SEQ ID NO: 401 - protein, 37 aa, Arabidopsis thaliana
VTYNSIIDAFGRSATMDRSADYSNGGSLPFSSSALSA

SEQ ID NO: 402 - protein, 58 aa, Arabidopsis thaliana
LTETEGNRVIQLFGQLTTESNNRTTKDCEEGMQELSCILEVFRKMHQLEIKPNVVTFS

SEQ ID NO: 403 - protein, 35 aa, Artificial Sequence
  MODIFIED TYPE GUN1
KLASAMISTLGRYGKVTIAKRIFETAFAGGIKNTV

SEQ ID NO: 404 - protein, 35 aa, Artificial Sequence
  MODIFIED TYPE GUN1
YAFSALISAYGRSGLHEEAISVFNSMKEYGIKPNL

SEQ ID NO: 405 - protein, 36 aa, Artificial Sequence
  MODIFIED TYPE GUN1
VTYNAVIDACGKGGMEFKQVAKFFDEMQRNGIKPDR

SEQ ID NO: 406 - protein, 35 aa, Artificial Sequence
  MODIFIED TYPE GUN1
ITFNSLLAVCSRGGLWEAARNLFDEMTNRRIKQDV

SEQ ID NO: 407 - protein, 35 aa, Artificial Sequence
  MODIFIED TYPE GUN1
FSYNTLLDAICKGGQMDLAFEILAQMPVKRIKPNV

SEQ ID NO: 408 - protein, 35 aa, Artificial Sequence
  MODIFIED TYPE GUN1
VSYSTVIDGFAKAGRFDEALNLFGEMRYLGIKLDR

SEQ ID NO: 409 - protein, 35 aa, Artificial Sequence
  MODIFIED TYPE GUN1
VTYNALLGGYGKQGKYDEVKKVFTEMKREHIKPNL

SEQ ID NO: 410 - protein, 35 aa, Artificial Sequence
  MODIFIED TYPE GUN1
LTYSTLIDGYSKGGLYKEAMEIFREFKSAGIKADV

SEQ ID NO: 411 - protein, 35 aa, Artificial Sequence
  MODIFIED TYPE GUN1
VLYSALIDALCKNGLVGSAVSLIDEMTKEGIKPNV

SEQ ID NO: 412 - protein, 37 aa, Artificial Sequence
  MODIFIED TYPE GUN1
VTYNSIIDAFGRSATMDRSADYSNGGSLPFSSIKLSA

SEQ ID NO: 413 - protein, 35 aa, Artificial Sequence
  MODIFIED TYPE GUN1
KLASAMISAYGRYGKVTKAKRIFETAFAGGIGNTV

SEQ ID NO: 414 - protein, 35 aa, Artificial Sequence
  MODIFIED TYPE GUN1
YAFSALISAYGRSGLHEKAISVFNSMKEYGIRPNL

SEQ ID NO: 415 - protein, 36 aa, Artificial Sequence
  MODIFIED TYPE GUN1
VTYNAVIDAYGKGGMEFKQVAKFFDEMQRNGIQPDR

SEQ ID NO: 416 - protein, 35 aa, Artificial Sequence
  MODIFIED TYPE GUN1
ITFNSLLAAYSRGGLWEKARNLFDEMTNRRIEQDV

SEQ ID NO: 417 - protein, 35 aa, Artificial Sequence
  MODIFIED TYPE GUN1
FSYNTLLDAYCKGGQMDKAFEILAQMPVKRIMPNV

SEQ ID NO: 418 - protein, 35 aa, Artificial Sequence
  MODIFIED TYPE GUN1
VSYSTVIDAYAKAGRFDKALNLFGEMRYLGIALDR

SEQ ID NO: 419 - protein, 35 aa, Artificial Sequence
  MODIFIED TYPE GUN1
VSYNTLLSAYTKVGRSEKALDILREMASVGIKKDV

SEQ ID NO: 420 - protein, 35 aa, Artificial Sequence
  MODIFIED TYPE GUN1
VTYNALLGAYGKQGKYDKVKKVFTEMKREHILPNL

SEQ ID NO: 421 - protein, 35 aa, Artificial Sequence
  MODIFIED TYPE GUN1
LTYSTLIDAYSKGGLYKKAMEIFREFKSAGIRADV

SEQ ID NO: 422 - protein, 35 aa, Artificial Sequence
  MODIFIED TYPE GUN1
VLYSALIDAYCKNGLVGKAVSLIDEMTKEGISPNV

SEQ ID NO: 423 - protein, 37 aa, Artificial Sequence
  MODIFIED TYPE GUN1
VTYNSIIDAYGRSATMDKSADYSNGGSLPFSSIALSA

SEQ ID NO: 424 - protein, 531 aa, Artificial Sequence
  MODIFIED TYPE P63
AGTKSDQEEDDLEDGFSELEGSKSGQGSTSSDEDEGKLSADEEEEEELDLIETDVSRKTVEKKQSELFKTIVSAPGLSIGSALDKWVEEGNEITRVEIAKAMLQLRRRRMYGRALQMSEWLEANKKIEMTERDYASRLDLTVKIRGLEKGEACMQKIPKSFKGEVLYRTLLANCVAAGNVKKSELVFNKMKDLGIKLSGFTCDQMLLLHKRIDRKKIADVLLLMEKENIKPSLLTYKILIDVKGATNDISGMEQILETMKDEGIKLDFQTQALTARHYSGAGLKDKAEKVLKEMEGESIKANRRAFKDLLSIYASLGREDEVKRIWKICEIKPYFEESLAAIQAFGKLNKVQEAEAIFEKIVKMDIKASSSTYSVLLRVYVDHKMLSKGKDLVKRMAESGIKIEATTWDALIKLYVEAGEVEKADSLLDKASKQSHIKLMMNSFMYIMDEYSKRGDVHNTEKIFLKMREAGIKSRLRQFQALMQAYINAKSPAYGMRDRLKADNIFPNKSMAAQLAQGDPFKKTAISDILD

SEQ ID NO: 425 - protein, 531 aa, Artificial Sequence
  MODIFIED TYPE P63
AGTKSDQEEDDLEDGFSELEGSKSGQGSTSSDEDEGKLSADEEEEEELDLIETDVSRKTVEKKQSELFKTIVSAPGLSIGSALDKWVEEGNEITRVEIAKAMLQLRRRRMYGRALQMSEWLEANKKIEMTERDYASRLDLTVKIRGLEKGEACMQKIPKSFKGEVLYRTLLAAYVAAGNVKKSELVFNKMKDLGIPLSGFTCDQMLLAYKRIDRKKKADVLLLMEKENIKPSLLTYKILIDAYGATNDISKMEQILETMKDEGIELDFQTQALTARAYSGAGLKDKAEKVLKEMEGESIEANRRAFKDLLSAYASLGREDKVKRIWKICEIKPYFEESLAAIQAYGKLNKVQKAEAIFEKIVKMDIRASSSTYSVLLRAYVDHKMLSKGKDLVKRMAESGIRIEATTWDALIKAYVEAGEVEKADSLLDKASKQSHIKLMMNSFMYIMDAYSKRGDVHKTEKIFLKMREAGITSRLRQFQALMQAYINAKSPAYGMRDRLKADNIFPNKSMAAQLAQGDPFKKTAISDILD

SEQ ID NO: 426 - protein, 669 aa, Artificial Sequence
  MODIFIED TYPE GUN1
SAHLSQTTPNFSPLQTPKSDFSGRQSTRFVSPATNNHRQTRQNPNYNHRPYGASSSPRGSAPPPSSVATVAPAQLSQPPNFSPLQTPKSDLSSDFSGRRSTRFVSKMHFGRQKTTMATRHSSAAEDALQNAIDFSGDDEMFHSLMLSFESKLCGSDDCTYIIRELGNRNECDKAVGFYEFAVKRERRKNEQGKLASAMISTLGRYGKVTIAKRIFETAFAGGIKNTVYAFSALISAYGRSGLHEEAISVFNSMKEYGIKPNLVTYNAVIDACGKGGMEFKQVAKFFDEMQRNGIKPDRITFNSLLAVCSRGGLWEAARNLFDEMTNRRIKQDVFSYNTLLDAICKGGQMDLAFEILAQMPVKRIKPNVVSYSTVIDGFAKAGRFDEALNLFGEMRYLGIKLDRVSYNTLLSIYTKVGRSEEALDILREMASVGIKKDVVTYNALLGGYGKQGKYDEVKKVFTEMKREHIKPNLLTYSTLIDGYSKGGLYKEAMEIFREFKSAGIKADVVLYSALIDALCKNGLVGSAVSLIDEMTKEGIKPNVVTYNSIIDAFGRSATMDRSADYSNGGSLPFSSIKLSALTETEGNRVIQLFGQLTTESNNRTTKDCEEGMQELSCILEVFRKMHQLEIKPNVVTFSAILNACSRCNSFEDASMLLEELRLFDNKVYG

SEQ ID NO: 427 - protein, 669 aa, Artificial Sequence
  MODIFIED TYPE GUN1
SAHLSQTTPNFSPLQTPKSDFSGRQSTRFVSPATNNHRQTRQNPNYNHRPYGASSSPRGSAPPPSSVATVAPAQLSQPPNFSPLQTPKSDLSSDFSGRRSTRFVSKMHFGRQKTTMATRHSSAAEDALQNAIDFSGDDEMFHSLMLSFESKLCGSDDCTYIIRELGNRNECDKAVGFYEFAVKRERRKNEQGKLASAMISAYGRYGKVTKAKRIFETAFAGGIGNTVYAFSALISAYGRSGLHEKAISVFNSMKEYGIRPNLVTYNAVIDAYGKGGMEFKQVAKFFDEMQRNGIQPDRITFNSLLAAYSRGGLWEKARNLFDEMTNRRIEQDVFSYNTLLDAYCKGGQMDKAFEILAQMPVKRIMPNVVSYSTVIDAYAKAGRFDKALNLFGEMRYLGIALDRVSYNTLLSAYTKVGRSEKALDILREMASVGIKKDVVTYNALLGAYGKQGKYDKVKKVFTEMKREHILPNLLTYSTLIDAYSKGGLYKKAMEIFREFKSAGIRADVVLYSALIDAYCKNGLVGKAVSLIDEMTKEGISPNVVTYNSIIDAYGRSATMDKSADYSNGGSLPFSSIALSALTETEGNRVIQLFGQLTTESNNRTTKDCEEGMQELSCILEVFRKMHQLEIKPNVVTFSAILNACSRCNSFEDASMLLEELRLFDNKVYG

(19) United States
(12) Patent Application Publication
(10) Pub. No.: US 2019/0177378 A1
(43) Pub. Date: Jun. 13, 2019

(54) DNA-BINDING PROTEIN USING PPR MOTIF, AND USE THEREOF

(71) Applicants: FUJIFILM Wako Pure Chemical Corporation, Osaka-shi, Osaka (JP); KYUSHU UNIVERSITY, NATIONAL UNIVERSITY CORPORATION, Fukuoka-shi, Fukuoka (JP)

(72) Inventors: Masayuki Yamane, Amagasaki-shi (JP); Takahiro Nakamura, Fukuoka-shi (JP); Yusuke Yagi, Fukuoka-shi (JP)

(73) Assignees: FUJIFILM Wako Pure Chemical Corporation, Osaka-shi, Osaka (JP); KYUSHU UNIVERSITY, NATIONAL UNIVERSITY CORPORATION, Fukuoka-shi, Fukuoka (JP)

(21) Appl. No.: 16/323,899

(22) PCT Filed: Aug. 9, 2017
(86) PCT No.: PCT/JP2017/028995

§ 371 Date: Feb. 7, 2019

(30) Foreign Application Priority Data
Aug. 10, 2016 (JP) 2016-157698

(51) Int. Cl. C07K 14/415; C12N 15/90

(52) U.S. Cl. CPC C07K 14/415; C12N 15/90

(57) ABSTRACT

The object of the present invention is to generalize and improve DNA-binding proteins using PPR. There is provided a protein that contains one or more PPR motifs having a structure of the following formula 1, wherein one PPR motif (Mₙ) contained in the protein is a PPR motif having a specific combination of amino acids corresponding to a target DNA base or target DNA base sequence as the three amino acids of No. 1 A.A., No. 4 A.A., and No. “ii” (−2) A.A, and satisfies at least one selected from the group consisting of the following conditions (a) to (h): (a) No. 7 A.A. of the PPR motif (Mₙ) is isoleucine (I); (b) No. 9 A.A. of the PPR motif (Mₙ) is alanine (A); (c) No. 10 A.A. of the PPR motif (Mₙ) is tyrosine (Y); (d) No. 18 A.A. of the PPR motif (Mₙ) is lysine (K), arginine (R), or histidine (H); (e) No. 20 A.A. of the PPR motif (Mₙ) is glutamic acid (E), or aspartic acid (D); (f) No. 29 A.A. of the PPR motif (Mₙ) is glutamic acid (E), or aspartic acid (D); (g) No. 31 A.A. of the PPR motif (Mₙ) is isoleucine (I); and (h) No. 32 A.A. of the PPR motif (Mₙ) is lysine (K), arginine (R), or histidine (H).

### TECHNICAL FIELD

The present invention relates to a protein that can selectively or specifically bind to an intended DNA base or DNA sequence. According to the present invention, a pentatricopeptide repeat (PPR) motif is utilized. The present invention can be used for identification and design of a DNA-binding protein, identification of a target DNA of a protein having a PPR motif, and functional control of DNA. The present invention is useful in the fields of medicine, agricultural science, and so forth. The present invention also relates to a novel DNA-cleaving enzyme that utilizes a complex of a protein containing a PPR motif and a protein that defines a functional region.

### BACKGROUND ART

In recent years, techniques of binding nucleic acid-binding protein factors elucidated through various analyses to an intended sequence have been established, and they are coming to be used. Use of this sequence-specific binding is enabling analysis of intracellular localization of a target nucleic acid (DNA or RNA), elimination of a target DNA sequence, or expression control (activation or inactivation) of a protein-encoding gene existing downstream of a target DNA sequence.

There are being conducted researches and developments using the zinc finger protein (Non-patent documents 1 and 2), TAL effecter (TALE, Non-patent document 3, Patent document 1), and CRISPR (Non-patent documents 4 and 5) as protein factors that act on DNA as materials for protein engineering. However, types of such protein factors are still extremely limited.

For example, the artificial enzyme, zinc finger nuclease (ZFN), known as an artificial DNA-cleaving enzyme, is a chimera protein obtained by binding a part that is constituted by linking 3 to 6 zinc fingers that specifically recognize a DNA consisting of 3 or 4 nucleotides and bind to it, and recognizes a nucleotide sequence in a sequence unit of 3 or 4 nucleotides with one DNA cleavage domain of a bacterial DNA-cleaving enzyme (for example, FokI) (Non-patent document 2). In such a chimera protein, the zinc finger domain is a protein domain that is known to bind to DNA, and it is based on the knowledge that many transcription factors have the aforementioned domain, and bind to a specific DNA sequence to control expression of a gene. By using two of ZFNs each having three zinc fingers, cleavage of one site per 70 billion nucleotides can be induced in theory.

However, because of the high cost required for the production of ZFNs, etc., the methods using ZFNs have not come to be widely used yet. Moreover, functional sorting efficiency of ZFNs is bad, and it is suggested that the methods have a problem also in this respect. Furthermore, since a zinc finger domain consisting of n of zinc fingers tends to recognize a sequence of (GNN)n, the methods also have a problem that degree of freedom for the target gene sequence is low.

An artificial enzyme, TALEN, has also been developed by binding a protein consisting of a combinatory sequence of module parts that can recognize every one nucleotide, TAL effecter (TALE), with a DNA cleavage domain of a bacterial DNA-cleaving enzyme (for example, FokI), and it is being investigated as an artificial enzyme that can replace ZFNs (Non-patent document 3). This TALEN is an enzyme generated by fusing a DNA binding domain of a transcription factor of a plant pathogenic Xanthomonas bacterium, and the DNA cleavage domain of the DNA restriction enzyme FokI, and it is known to bind to a neighboring DNA sequence to form a dimer and cleave a double strand DNA. Since, as for this molecule, the DNA binding domain of TALE found from a bacterium that infects with plants recognize one base with a combination of amino acids at two sites in the TALE motif consisting of 34 amino acid residues, it has a characteristic that binding property for a target DNA can be chosen by choosing the repetitive structure of the TALE module. TALEN using the DNA binding domain that has such a characteristic as mentioned above has a characteristic that it enables introduction of mutation into a target gene, like ZFNs, but the significant superiority thereof to ZFNs is that degree of freedom for the target gene (nucleotide sequence) is markedly improved, and the nucleotide to which it binds can be defined with a code.

However, since the total conformation of TALEN has not been elucidated, the DNA cleavage site of TALEN has not been identified at present. Therefore, it has a problem that cleavage site of TALEN is inaccurate, and is not fixed, compared with ZFNs, and it also cleaves even a similar sequence. Therefore, it has a problem that a nucleotide sequence cannot be accurately cleaved at an intended target site with a DNA-cleaving enzyme. For these reasons, it is desired to develop and provide a novel artificial DNA-cleaving enzyme free from the aforementioned problems.

On the basis of genome sequence information, PPR proteins (proteins having a pentatricopeptide repeat (PPR) motif) constituting a big family of no less than 500 members only for plants have been identified (Non-patent document 6). The PPR proteins are nucleus-encoded proteins, but are known to act on or involved in control, cleavage, translation, splicing, RNA editing, and RNA stability chiefly at an RNA level in organelles (chloroplasts and mitochondria) in a gene-specific manner. The PPR proteins typically have a structure consisting of about 10 contiguous 35-amino acid motifs of low conservativeness, i.e., PPR motifs, and it is considered that the combination of the PPR motifs is responsible for the sequence-selective binding with RNA. Almost all the PPR proteins consist only of repetition of about 10 PPR motifs, and any domain required for exhibiting a catalytic action is not found in many cases. Therefore, it is considered that the PPR proteins are essentially RNA adapters (Non-patent document 7).

In general, binding of a protein and DNA, and binding of a protein and RNA are attained by different molecular mechanisms. Therefore, a DNA-binding protein generally does not bind to RNA, whereas an RNA-binding protein generally does not bind to DNA. For example, in the case of the pumilio protein, which is known as an RNA-binding factor, and can encode RNA to be recognized, binding thereof to DNA has not been reported (Non-patent documents 8 and 9).

However, in the process of investigating properties of various kinds of PPR proteins, it became clear that it could be suggested that some types of the PPR proteins worked as DNA-binding factors.

On the other hand, the wheat p63 is a PPR protein having 9 PPR motifs, and it has been suggested that it binds with DNA in a sequence-specific manner, which has been proven by gel shift assay (Non-patent document 10). The GUN1 protein of Arabidopsis thaliana has 11 PPR motifs, and it has been suggested that it binds with DNA, which has been proven by pull-down assay (Non-patent document 11). It has been demonstrated by run-on assay that the Arabidopsis thaliana pTac2 (protein having 15 PPR motifs, Non-patent document 12) and Arabidopsis thaliana DG1 (protein having 10 PPR motifs, Non-patent document 13) directly participate in transcription for generating RNA by using DNA as a template, and they are considered to bind with DNA. An Arabidopsis thaliana strain deficient in the gene of GRP23 (protein having 11 PPR motifs, Non-patent document 14) shows a phenotype of embryonal death. It has been demonstrated that this protein physically interacts with the major subunit of the eukaryotic RNA transcription polymerase 2, which is a DNA-dependent RNA transcription enzyme, and therefore it is considered that GRP23 also acts in binding with DNA. The inventors of the present invention analyzed the structures and functions of p63 of wheat, GUN1 of Arabidopsis thaliana, pTac2 of Arabidopsis thaliana, DG1 of Arabidopsis thaliana, and so forth with a prediction that the RNA recognition rules of the PPR motifs can also be applied to the recognition of DNA, and proposed a method for designing a custom-made DNA-binding protein that binds to a desired sequence (Patent document 4).

### PRIOR ART REFERENCES

### Patent Documents

Patent document 1: WO2011/072246
Patent document 2: WO2011/111829
Patent document 3: WO2013/058404
Patent document 4: WO2014/175284

### Non-Patent Documents

Non-patent document 1: Maeder, M. L., et al. (2008) Rapid “open-source” engineering of customized zinc-finger nucleases for highly efficient gene modification, Mol. Cell 31, 294-301
Non-patent document 2: Urnov, F. D., et al. (2010) Genome editing with engineered zinc finger nucleases, Nature Review Genetics, 11, 636-646
Non-patent document 3: Miller, J. C., et al. (2011) A TALE nuclease architecture for efficient genome editing, Nature Biotech., 29, 143-148
Non-patent document 4: Mali P., et al. (2013) RNA-guided human genome engineering via Cas9, Science, 339, 823-826
Non-patent document 5: Cong L., et al. (2013) Multiplex genome engineering using CRISPR/Cas systems, Science, 339, 819-823
Non-patent document 6: Small, I. D. and Peeters, N. (2000) The PPR motif—a TPR-related motif prevalent in plant organellar proteins, Trends Biochem. Sci., 25, 46-47
Non-patent document 7: Woodson, J. D., and Chory, J. (2008) Coordination of gene expression between organellar and nuclear genomes, Nature Rev. Genet., 9, 383-395
Non-patent document 8: Wang, X., et al. (2002) Modular recognition of RNA by a human pumilio-homology domain, Cell, 110, 501-512
Non-patent document 9: Cheong, C. G., and Hall and T. M. (2006) Engineering RNA sequence specificity of Pumilio repeats, Proc. Natl. Acad. Sci. USA 103, 13635-13639
Non-patent document 10: Ikeda T. M. and Gray M. W. (1999) Characterization of a DNA-binding protein implicated in transcription in wheat mitochondria, Mol. Cell Bio., 119 (12):8113-8122
Non-patent document 11: Koussevitzky S., et al. (2007) Signals from chloroplasts converge to regulate nuclear gene expression, Science, 316:715-719
Non-patent Document 12: Pfalz J, et al. (2006) PTAC2, −6, and −12 are components of the transcriptionally active plastid chromosome that are required for plastid gene expression, Plant Cell 18:176-197
Non-patent document 13: Chi W, et al. (2008) The pentatricopeptide repeat protein DELAYED GREENING1 is involved in the regulation of early chloroplast development and chloroplast gene expression in Arabidopsis, Plant Physiol., 147:573-584
Non-patent document 14: Ding Y H, et al. (2006) Arabidopsis GLUTAMINE-RICH PROTEIN 23 is essential for early embryogenesis and encodes a novel nuclear PPR motif protein that interacts with RNA polymerase II subunit III, Plant Cell, 18:815-830

### SUMMARY OF THE INVENTION

### Object to be Achieved by the Invention

As actual dPPR proteins (DNA-binding proteins using PPR), there are only P63, GUN1, _{P}TAC2, GRP23, and DG1 described in Patent document 4, and it is hard to say that they are sufficient for acquiring information for generalizing and improving the artificial nucleic acid-binding modules based on the PPR techniques.

### Means for Achieving the Object

Therefore, the inventors of the present invention decided to perform screening for searching PPR proteins having a DNA-binding ability to increase dPPR proteins. While the genes of the dPPR proteins accidentally found so far contain an intron, almost all the genes of rPPR proteins (RNA-binding proteins using PPR) do not have any intron. When the total genome sequences of the model plant, Arabidopsis thaliana, were analyzed by using the aforementioned fact as an index, there were found 42 types of PPR genes containing two or more introns. The inventors of the present invention analyzed the DNA-binding abilities of these 42 kinds of potential dPPR molecules to attempt to identify novel dPPR molecules. On the basis of the amino acid sequence information of the modules of the identified dPPR proteins, they also analyzed dPPR motif-specific amino acid sequences. They further investigated the DNA-binding abilities of modified type rPPRs containing a dPPR-specific amino acid sequence in order to verify whether the DNA-binding ability of PPR protein is increased by a dPPR-specific amino acid sequence. As a result, they accomplished the present invention.

The present invention provides the followings.
[1] A protein that can bind in a DNA base-selective manner or a DNA base sequence-specific manner, which contains one or more PPR motifs having a structure of the following formula 1:

[Chemical Formula 1]

(Helix A)-X-(Helix B)-L (Formula 1)

(wherein, in the formula 1:
Helix A is a part that can form an α-helix structure;
X does not exist, or is a part consisting of 1 to 9 amino acids;
Helix B is a part that can form an α-helix structure; and
L is a part consisting of 2 to 7 amino acids),
wherein,
under the following definitions:
the first amino acid of Helix A is referred to as No. 1 amino acid (No. 1 A.A.), the fourth amino acid as No. 4 amino acid (No. 4 A.A.), and
when a next PPR motif (Mₙ₊₁) contiguously exists on the C-terminus side of the PPR motif (Mₙ) (when there is no amino acid insertion between the PPR motifs), the −2nd amino acid counted from the end (C-terminus side) of the amino acids constituting the PPR motif (Mₙ);
when a non-PPR motif consisting of 1 to 20 amino acids exists between the PPR motif (Mₙ) and the next PPR motif (Mₙ₊₁) on the C-terminus side, the amino acid locating upstream of the first amino acid of the next PPR motif (Mₙ₊₁) by 2 positions, i.e., the −2nd amino acid; or
when any next PPR motif (Mₙ₊₁) does not exist on the C-terminus side of the PPR motif (Mₙ), or 21 or more amino acids constituting a non-PPR motif exist between the PPR motif (Mₙ) and the next PPR motif (Mₙ₊₁) on the C-terminus side, the 2nd amino acid counted from the end (C-terminus side) of the amino acids constituting the PPR motif (Mₙ)
is referred to as No. “ii” (−2) amino acid (No. “ii” (−2) A.A.),
one PPR motif (Mₙ) contained in the protein is a PPR motif having a specific combination of amino acids corresponding to a target DNA base or target DNA base sequence as the three amino acids of No. 1 A.A., No. 4 A.A., and No. “ii” (-2) A.A, and the protein satisfies at least one selected from the group consisting of the following conditions (a) to (h), preferably (b) to (h):
(a) No. 7 A.A. of the PPR motif (Mₙ) is isoleucine (I);
(b) No. 9 A.A. of the PPR motif (Mₙ) is alanine (A);
(c) No. 10 A.A. of the PPR motif (Mₙ) is tyrosine (Y), phenylalanine (F), or tryptophan (W);
(d) No. 18 A.A. of the PPR motif (Mₙ) is lysine (K), arginine (R), or histidine (H);
(e) No. 20 A.A. of the PPR motif (Mₙ) is glutamic acid (E), or aspartic acid (D);
(f) No. 29 A.A. of the PPR motif (MO is glutamic acid (E), or aspartic acid (D);
(g) No. 31 A.A. of the PPR motif (MO is isoleucine (I), leucine (L), or valine (V); and
(h) No. 32 A.A. of the PPR motif (MO is lysine (K), arginine (R), or histidine (H) (provided that a protein consisting of any one of the amino acid sequences of SEQ ID NOS: 1 to 5 and SEQ ID NOS: 291 to 308 is excluded).
[2] The protein according to [1], wherein the combination of the three amino acids of No. 1 A.A., No. 4 A.A., and No. “ii” (−2) A.A. is a combination corresponding to a target DNA base or target DNA base sequence, and the combination of amino acids is determined according to any one of the following definitions:
(1-1) when No. 4 A.A. is glycine (G), No. 1 A.A. may be an arbitrary amino acid, and No. “ii” (-2) A.A. is aspartic acid (D), asparagine (N), or serine (S);
(1-2) when No. 4 A.A. is isoleucine (I), each of No. 1 A.A. and No. “ii” (−2) A.A. may be an arbitrary amino acid;
(1-3) when No. 4 A.A. is leucine (L), each of No. 1 A.A. and No. “ii” (−2) A.A. may be an arbitrary amino acid;
(1-4) when No. 4 A.A. is methionine (M), each of No. 1 A.A. and No. “ii” (−2) A.A. may be an arbitrary amino acid;
(1-5) when No. 4 A.A. is asparagine (N), each of No. 1 A.A. and No. “ii” (−2) A.A. may be an arbitrary amino acid;
(1-6) when No. 4 A.A. is proline (P), each of No. 1 A.A. and No. “ii” (−2) A.A. may be an arbitrary amino acid;
(1-7) when No. 4 A.A. is serine (S), each of No. 1 A.A. and No. “ii” (−2) A.A. may be an arbitrary amino acid;
(1-8) when No. 4 A.A. is threonine (T), each of No. 1 A.A. and No. “ii” (−2) A.A. may be an arbitrary amino acid; and
(1-9) when No. 4 A.A. is valine (V), each of No. 1 A.A. and No. “ii” (−2) A.A. may be an arbitrary amino acid.
[3] The protein according to [1], wherein the combination of the three amino acids of No. 1 A.A., No. 4 A.A., and No. “ii” (−2) A.A. is a combination corresponding to a target DNA base or target DNA base sequence, and the combination of amino acids is determined according to any one of the following definitions:
(2-1) when the three amino acids, No. 1 A.A., No. 4 A.A., and No. “ii” (−2) A.A. are an arbitrary amino acid, glycine, and aspartic acid, respectively, the PPR motif selectively binds to G;
(2-2) when the three amino acids, No. 1 A.A., No. 4 A.A., and No. “ii” (−2) A.A., are glutamic acid, glycine, and aspartic acid, respectively, the PPR motif selectively binds to G;
(2-3) when the three amino acids, No. 1 A.A., No. 4 A.A., and No. “ii” (−2) A.A., are an arbitrary amino acid, glycine, and asparagine, respectively, the PPR motif selectively binds to A;
(2-4) when the three amino acids, No. 1 A.A., No. 4 A.A., and No. “ii” (−2) A.A., are glutamic acid, glycine, and asparagine, respectively, the PPR motif selectively binds to A;
(2-5) when the three amino acids, No. 1 A.A., No. 4 A.A., and No. “ii” (−2) A.A., are an arbitrary amino acid, glycine, and serine, respectively, the PPR motif selectively binds to A, and next binds to C;
(2-6) when the three amino acids, No. 1 A.A., No. 4 A.A., and No. “ii” (−2) A.A., are an arbitrary amino acid, isoleucine, and an arbitrary amino acid, respectively, the PPR motif selectively binds to T and C;
(2-7) when the three amino acids, No. 1 A.A., No. 4 A.A., and No. “ii” (−2) A.A., are an arbitrary amino acid, isoleucine, and asparagine, respectively, the PPR motif selectively binds to T, and next binds to C;
(2-8) when the three amino acids, No. 1 A.A., No. 4 A.A., and No. “ii” (−2) A.A., are an arbitrary amino acid, leucine, and an arbitrary amino acid, respectively, the PPR motif selectively binds to T and C;
(2-9) when the three amino acids, No. 1 A.A., No. 4 A.A., and No. “ii” (−2) A.A., are an arbitrary amino acid, leucine, and aspartic acid, respectively, the PPR motif selectively binds to C;
(2-10) when the three amino acids, No. 1 A.A., No. 4 A.A., and No. “ii” (−2) A.A., are an arbitrary amino acid, leucine, and lysine, respectively, the PPR motif selectively binds to T;
(2-11) when the three amino acids, No. 1 A.A., No. 4 A.A., and No. “ii” (−2) A.A., are an arbitrary amino acid, methionine, and an arbitrary amino acid, respectively, the PPR motif selectively binds to T;
(2-12) when the three amino acids, No. 1 A.A., No. 4 A.A., and No. “ii” (−2) A.A., are an arbitrary amino acid, methionine, and aspartic acid, respectively, the PPR motif selectively binds to T;
(2-13) when the three amino acids, No. 1 A.A., No. 4 A.A., and No. “ii” (−2) A.A., are isoleucine, methionine, and aspartic acid, respectively, the PPR motif selectively binds to T, and next binds to C;
(2-14) when the three amino acids, No. 1 A.A., No. 4 A.A., and No. “ii” (−2) A.A., are an arbitrary amino acid, asparagine, and an arbitrary amino acid, respectively, the PPR motif selectively binds to C and T;
(2-15) when the three amino acids, No. 1 A.A., No. 4 A.A., and No. “ii” (−2) A.A., are an arbitrary amino acid, asparagine, and aspartic acid, respectively, the PPR motif selectively binds to T;
(2-16) when the three amino acids, No. 1 A.A., No. 4 A.A., and No. “ii” (−2) A.A., are phenylalanine, asparagine, and aspartic acid, respectively, the PPR motif selectively binds to T;
(2-17) when the three amino acids, No. 1 A.A., No. 4 A.A., and No. “ii” (−2) A.A., are glycine, asparagine, and aspartic acid, respectively, the PPR motif selectively binds to T;
(2-18) when the three amino acids, No. 1 A.A., No. 4 A.A., and No. “ii” (−2) A.A., are isoleucine, asparagine, and aspartic acid, respectively, the PPR motif selectively binds to T;
(2-19) when the three amino acids, No. 1 A.A., No. 4 A.A., and No. “ii” (−2) A.A., are threonine, asparagine, and aspartic acid, respectively, the PPR motif selectively binds to T;
(2-20) when the three amino acids, No. 1 A.A., No. 4 A.A., and No. “ii” (−2) A.A. are valine, asparagine, and aspartic acid, respectively, the PPR motif selectively binds to T, and next binds to C;
(2-21) when the three amino acids, No. 1 A.A., No. 4 A.A., and No. “ii” (−2) A.A. are tyrosine, asparagine, and aspartic acid, respectively, the PPR motif selectively binds to T, and next binds to C;
(2-22) when the three amino acids, No. 1 A.A., No. 4 A.A., and No. “ii” (−2) A.A., are an arbitrary amino acid, asparagine, and asparagine, respectively, the PPR motif selectively binds to C;
(2-23) when the three amino acids, No. 1 A.A., No. 4 A.A., and No. “ii” (−2) A.A., are isoleucine, asparagine, and asparagine, respectively, the PPR motif selectively binds to C;
(2-24) when the three amino acids, No. 1 A.A., No. 4 A.A., and No. “ii” (−2) A.A., are serine, asparagine, and asparagine, respectively, the PPR motif selectively binds to C;
(2-25) when the three amino acids, No. 1 A.A., No. 4 A.A., and No. “ii” (−2) A.A., are valine, asparagine, and asparagine, respectively, the PPR motif selectively binds to C;
(2-26) when the three amino acids, No. 1 A.A., No. 4 A.A., and No. “ii” (−2) A.A., are an arbitrary amino acid, asparagine, and serine, respectively, the PPR motif selectively binds to C;
(2-27) when the three amino acids, No. 1 A.A., No. 4 A.A., and No. “ii” (−2) A.A., are valine, asparagine, and serine, respectively, the PPR motif selectively binds to C;
(2-28) when the three amino acids, No. 1 A.A., No. 4 A.A., and No. “ii” (−2) A.A., are an arbitrary amino acid, asparagine, and threonine, respectively, the PPR motif selectively binds to C;
(2-29) when the three amino acids, No. 1 A.A., No. 4 A.A., and No. “ii” (−2) A.A., are valine, asparagine, and threonine, respectively, the PPR motif selectively binds to C;
(2-30) when the three amino acids, No. 1 A.A., No. 4 A.A., and No. “ii” (−2) A.A., are an arbitrary amino acid, asparagine, and tryptophan, respectively, the PPR motif selectively binds to C, and next binds to T;
(2-31) when the three amino acids, No. 1 A.A., No. 4 A.A., and No. “ii” (−2) A.A., are isoleucine, asparagine, and tryptophan, respectively, the PPR motif selectively binds to T, and next binds to C;
(2-32) when the three amino acids, No. 1 A.A., No. 4 A.A., and No. “ii” (−2) A.A., are an arbitrary amino acid, proline, and an arbitrary amino acid, respectively, the PPR motif selectively binds to T;
(2-33) when the three amino acids, No. 1 A.A., No. 4 A.A., and No. “ii” (−2) A.A., are an arbitrary amino acid, proline, and aspartic acid, respectively, the PPR motif selectively binds to T;
(2-34) when the three amino acids, No. 1 A.A., No. 4 A.A., and No. “ii” (−2) A.A., are phenylalanine, proline, and aspartic acid, respectively, the PPR motif selectively binds to T;
(2-35) when the three amino acids, No. 1 A.A., No. 4 A.A., and No. “ii” (−2) A.A., are tyrosine, proline, and aspartic acid, respectively, the PPR motif selectively binds to T;
(2-36) when the three amino acids, No. 1 A.A., No. 4 A.A., and No. “ii” (−2) A.A., are an arbitrary amino acid, serine, and an arbitrary amino acid, respectively, the PPR motif selectively binds to A and G;
(2-37) when the three amino acids, No. 1 A.A., No. 4 A.A., and No. “ii” (−2) A.A., are an arbitrary amino acid, serine, and asparagine, respectively, the PPR motif selectively binds to A;
(2-38) when the three amino acids, No. 1 A.A., No. 4 A.A., and No. “ii” (−2) A.A., are phenylalanine, serine, and asparagine, respectively, the PPR motif selectively binds to A;
(2-39) when the three amino acids, No. 1 A.A., No. 4 A.A., and No. “ii” (−2) A.A., are valine, serine, and asparagine, respectively, the PPR motif selectively binds to A;
(2-40) when the three amino acids, No. 1 A.A., No. 4 A.A., and No. “ii” (−2) A.A., are an arbitrary amino acid, threonine, and an arbitrary amino acid, respectively, the PPR motif selectively binds to A and G;
(2-41) when the three amino acids, No. 1 A.A., No. 4 A.A., and No. “ii” (−2) A.A., are an arbitrary amino acid, threonine, and aspartic acid, respectively, the PPR motif selectively binds to G;
(2-42) when the three amino acids, No. 1 A.A., No. 4 A.A., and No. “ii” (−2) A.A., are valine, threonine, and aspartic acid, respectively, the PPR motif selectively binds to G;
(2-43) when the three amino acids, No. 1 A.A., No. 4 A.A., and No. “ii” (−2) A.A., are an arbitrary amino acid, threonine, and asparagine, respectively, the PPR motif selectively binds to A;
(2-44) when the three amino acids, No. 1 A.A., No. 4 A.A., and No. “ii” (−2) A.A., are phenylalanine, threonine, and asparagine, respectively, the PPR motif selectively binds to A;
(2-45) when the three amino acids, No. 1 A.A., No. 4 A.A., and No. “ii” (−2) A.A., are isoleucine, threonine, and asparagine, respectively, the PPR motif selectively binds to A;
(2-46) when the three amino acids, No. 1 A.A., No. 4 A.A., and No. “ii” (−2) A.A., are valine, threonine, and asparagine, respectively, the PPR motif selectively binds to A;
(2-47) when the three amino acids, No. 1 A.A., No. 4 A.A., and No. “ii” (−2) A.A., are an arbitrary amino acid, valine, and an arbitrary amino acid, respectively, the PPR motif binds with A, C, and T, but does not bind to G;
(2-48) when the three amino acids, No. 1 A.A., No. 4 A.A., and No. “ii” (−2) A.A., are isoleucine, valine, and aspartic acid, respectively, the PPR motif selectively binds to C, and next binds to A;
(2-49) when the three amino acids, No. 1 A.A., No. 4 A.A., and No. “ii” (−2) A.A., are an arbitrary amino acid, valine, and glycine, respectively, the PPR motif selectively binds to C; and
(2-50) when the three amino acids, No. 1 A.A., No. 4 A.A., and No. “ii” (−2) A.A., are an arbitrary amino acid, valine, and threonine, respectively, the PPR motif selectively binds to T.
[4] The protein according to any one of [1] to [3], which contains 2 to 30 of the PPR motifs (Mₙ) defined in [1].
[5] The protein according to any one of [1] to [4], which satisfies at least one selected from the group consisting of the combination of (b) and (c), the combination of (d) and (e), (a), (g), and (h), preferably the protein according to any one of [1] to [4], which satisfies at least one selected from the group consisting of the combination of (b) and (c), the combination of (d) and (e), (g), and (h).
[6] The protein according to [5], which satisfies the combination of (b) and (c), and satisfies at least one selected from the group consisting of the combination of (d) and (e), (a), (g), and (h), preferably the protein according to [5], which satisfies the combination of (b) and (c), and satisfies at least one selected from the group consisting of the combination of (d) and (e), (g), and (h).
[7] The protein according to [6], which satisfies the combination of (b) and (c), the combination of (d) and (e), (a), and (g), preferably the protein according to [6], which satisfies the combination of (b) and (c), the combination of (d) and (e), and (g).
[8] The protein according to any one of [1] to [7], which contains a plurality of PPR motifs, and satisfies any of the following (i) to (viii):
(i) at least 40% of No. 7 A.A. consists of isoleucine (I);
(ii) at least 36% of No. 9 A.A. consists of alanine (A);
(iii) at least 37% of No. 10 A.A. consists of tyrosine (Y), phenylalanine (F), or tryptophan (W);
(iv) at least 19% of No. 18 A.A. consists of lysine (K), arginine (R), or histidine (H);
(v) at least 21% of No. 20 A.A. consists of glutamic acid (E) or aspartic acid (D);
(vi) at least 9% of No. 29 A.A. consists of glutamic acid (E) or aspartic acid (D);
(vii) at least 16% of No. 31 A.A. consists of isoleucine (I), leucine (L), or valine (V);
(viii) at least 15% of No. 32 A.A. consists of lysine (K), arginine (R), or histidine (H), or

the protein according to any one of [1] to [7], which contains a plurality of PPR motifs, and has a DNA-binding PPR motif content of 13% or higher.
[9] A protein consisting of:

any one of the amino acid sequences of SEQ ID NOS: 7 to 214;

any one amino acid sequence selected from the group consisting of the amino acid sequence of the 167 to 482 positions of SEQ ID NO: 291, the amino acid sequence of the 156 to 575 positions of SEQ ID NO: 292, the amino acid sequence of the 243 to 554 positions of SEQ ID NO: 293, the amino acid sequence of the 140 to 489 positions of SEQ ID NO: 294, the amino acid sequence of the 78 to 419 positions of SEQ ID NO: 295, the amino acid sequence of the 122 to 545 positions of SEQ ID NO: 296, the amino acid sequence of the 256 to 624 positions of SEQ ID NO: 297, the amino acid sequence of the 48 to 362 positions of SEQ ID NO: 298, the amino acid sequence of the 198 to 689 positions of SEQ ID NO: 299, the amino acid sequence of the 89 to 578 positions of SEQ ID NO: 300, the amino acid sequence of the 470 to 911 positions of SEQ ID NO: 301, the amino acid sequence of the 156 to 575 positions of SEQ ID NO: 302, the amino acid sequence of the 108 to 775 positions of SEQ ID NO: 303, the amino acid sequence of the 226 to 1137 positions of SEQ ID NO: 304, the amino acid sequence of the 145 to 496 positions of SEQ ID NO: 305, the amino acid sequence of the 104 to 538 positions of SEQ ID NO: 306, the amino acid sequence of the 151 to 502 positions of SEQ ID NO: 307, and the amino acid sequence of the 274 to 660 positions of SEQ ID NO: 308;

any one of the amino acid sequences of SEQ ID NOS: 335 to 361; or

any one of the amino acid sequences of SEQ ID NOS: 424 to 427.
[10] A complex consisting of
   a region consisting of
   the protein according to any one of [1] to [9], or a protein consisting of any one of the amino acid sequences of SEQ ID NOS: 291 to 308, or a part thereof;
   a protein consisting of any one of the amino acid sequences of SEQ ID NOS: 335 to 361; or
   a protein consisting of any one of the amino acid sequences of SEQ ID NOS: 424 to 427, and
      a functional region bound together.
[11] The complex according to [10], wherein the functional region is fused to the protein on the C-terminus side of the protein.
[12] The complex according to [10] or [11], wherein the functional region is a DNA-cleaving enzyme, or a nuclease domain thereof, or a transcription control domain, and the complex functions as a target sequence-specific DNA-cleaving enzyme or transcription control factor.
[13] The complex according to [12], wherein the DNA-cleaving enzyme is the nuclease domain of FokI (SEQ ID NO: 6).
[14] A method for designing a protein that binds to a DNA base or DNA having a specific base sequence, which comprises replacing one or two or more amino acids on the basis of any one selected from the group consisting of (a) to (h), preferably (b) to (h), defined in [1] in any of:

a protein having any one amino acid sequence selected from the group consisting of the amino acid sequence of the 230 to 541 positions of SEQ ID NO: 1, the amino acid sequence of the 234 to 621 positions of SEQ ID NO: 2, the amino acid sequence of the 106 to 632 positions of SEQ ID NO: 3, the amino acid sequence of the 106 to 632 positions of SEQ ID NO: 4, and the amino acid sequence of the 256 to 624 positions of SEQ ID NO: 5;

any one PPR motif selected from the group consisting of 9 PPR motifs of the protein consisting of the amino acid sequence of SEQ ID NO: 1, 11 PPR motifs of the protein consisting of the amino acid sequence of SEQ ID NO: 2, 15 PPR motifs of the protein consisting of the amino acid sequence of SEQ ID NO: 3, 10 PPR motifs of the protein consisting of the amino acid sequence of SEQ ID NO: 4, and 11 PPR motifs of the protein consisting of the amino acid sequence of SEQ ID NO: 5;

a protein having any one amino acid sequence selected from the group consisting of the amino acid sequence of the 167 to 482 positions of SEQ ID NO: 291, the amino acid sequence of the 156 to 575 positions of SEQ ID NO: 292, the amino acid sequence of the 243 to 554 positions of SEQ ID NO: 293, the amino acid sequence of the 140 to 489 positions of SEQ ID NO: 294, the amino acid sequence of the 78 to 419 positions of SEQ ID NO: 295, the amino acid sequence of the 122 to 545 positions of SEQ ID NO: 296, the amino acid sequence of the 256 to 624 positions of SEQ ID NO: 297, the amino acid sequence of the 48 to 362 positions of SEQ ID NO: 298, the amino acid sequence of the 198 to 689 positions of SEQ ID NO: 299, the amino acid sequence of the 89 to 578 positions of SEQ ID NO: 300, the amino acid sequence of the 470 to 911 positions of SEQ ID NO: 301, the amino acid sequence of the 156 to 575 positions of SEQ ID NO: 302, the amino acid sequence of the 108 to 775 positions of SEQ ID NO: 303, the amino acid sequence of the 226 to 1137 positions of SEQ ID NO: 304, the amino acid sequence of the 145 to 496 positions of SEQ ID NO: 305, the amino acid sequence of the 104 to 538 positions of SEQ ID NO: 306, the amino acid sequence of the 151 to 502 positions of SEQ ID NO: 307, and the amino acid sequence of the 274 to 660 positions of SEQ ID NO: 308, and

any one PPR motif selected from the group consisting of 9 PPR motifs of the protein consisting of the amino acid sequence SEQ ID NO: 291, 6 PPR motifs of the protein consisting of the amino acid sequence of SEQ ID NO: 292, 9 PPR motifs of the protein consisting of the amino acid sequence of SEQ ID NO: 293, 10 PPR motifs of the protein consisting of the amino acid sequence of SEQ ID NO: 294, 9 PPR motifs of the protein consisting of the amino acid sequence of SEQ ID NO: 295, 12 PPR motifs of the protein consisting of the amino acid sequence of SEQ ID NO: 296, 10 PPR motifs of the protein consisting of the amino acid sequence of SEQ ID NO: 297, 9 PPR motifs of the protein consisting of the amino acid sequence of SEQ ID NO: 298, 14 PPR motifs of the protein consisting of the amino acid sequence of SEQ ID NO: 299, 14 PPR motifs of the protein consisting of the amino acid sequence of SEQ ID NO: 300, 10 PPR motifs of the protein consisting of the amino acid sequence of SEQ ID NO: 301, 12 PPR motifs of the protein consisting of the amino acid sequence of SEQ ID NO: 302, 19 PPR motifs of the protein consisting of the amino acid sequence of SEQ ID NO: 303, 25 PPR motifs of the protein consisting of the amino acid sequence of SEQ ID NO: 304, 10 PPR motifs of the protein consisting of the amino acid sequence of SEQ ID NO: 305, 9 PPR motifs of the protein consisting of the amino acid sequence of SEQ ID NO: 306, 10 PPR motifs of the protein consisting of the amino acid sequence of SEQ ID NO: 307, and 11 PPR motifs of the protein consisting of the amino acid sequence of SEQ ID NO: 308.
[15] A method for designing a protein that binds to a DNA base or DNA having a specific base sequence, which comprises making the protein contain one or more PPR motifs having a structure of the following formula 1:

[Chemical Formula 2]

(Helix A)-X-(Helix B)-L (Formula 1)

(wherein, in the formula 1:
Helix A is a part that can form an α-helix structure;
X does not exist, or is a part consisting of 1 to 9 amino acids;
Helix B is a part that can form an α-helix structure; and
L is a part consisting of 2 to 7 amino acids),
wherein,
under the following definitions:
the first amino acid of Helix A is referred to as No. 1 amino acid (No. 1 A.A.), the fourth amino acid as No. 4 amino acid (No. 4 A.A.), and
when a next PPR motif (Mₙ₊₁) contiguously exists on the C-terminus side of the PPR motif (Mₙ) (when there is no amino acid insertion between the PPR motifs), the −2nd amino acid counted from the end (C-terminus side) of the amino acids constituting the PPR motif (Mₙ);
when a non-PPR motif consisting of 1 to 20 amino acids exists between the PPR motif (Mₙ) and the next PPR motif (Mₙ₊₁) on the C-terminus side, the amino acid locating upstream of the first amino acid of the next PPR motif (Mₙ₊₁) by 2 positions, i.e., the −2nd amino acid; or
when any next PPR motif (Mₙ₊₁) does not exist on the C-terminus side of the PPR motif (Mₙ), or 21 or more amino acids constituting a non-PPR motif exist between the PPR motif (Mₙ) and the next PPR motif (Mₙ₊₁) on the C-terminus side, the 2nd amino acid counted from the end (C-terminus side) of the amino acids constituting the PPR motif (Mₙ)
is referred to as No. “ii” (−2) amino acid (No. “ii” (−2) A.A.),
one PPR motif (Mₙ) contained in the protein is a PPR motif having a specific combination of amino acids corresponding to a target DNA base or target DNA base sequence as the three amino acids of No. 1 A.A., No. 4 A.A., and No. “ii” (−2) A.A, and satisfies at least one selected from the group consisting of the following conditions (a) to (h), preferably (b) to (h):
(a) No. 7 A.A. of the PPR motif (Mₙ) is isoleucine (I);
(b) No. 9 A.A. of the PPR motif (Mₙ) is alanine (A);
(c) No. 10 A.A. of the PPR motif (Mₙ) is tyrosine (Y), phenylalanine (F), or tryptophan (W);
(d) No. 18 A.A. of the PPR motif (Mₙ) is lysine (K), arginine (R), or histidine (H);
(e) No. 20 A.A. of the PPR motif (Mₙ) is glutamic acid (E), or aspartic acid (D);
(f) No. 29 A.A. of the PPR motif (Mₙ) is glutamic acid (E), or aspartic acid (D);
(g) No. 31 A.A. of the PPR motif (Mₙ) is isoleucine (I), leucine (L), or valine (V); and
(h) No. 32 A.A. of the PPR motif (Mₙ) is lysine (K), arginine (R), or histidine (H).
[16] The method according to [15], wherein the combination of the three amino acids of No. 1 A.A., No. 4 A.A., and No. “ii” (−2) A.A. is determined according to any one of the following definitions:
(1-1) when No. 4 A.A. is glycine (G), No. 1 A.A. may be an arbitrary amino acid, and No. “ii” (−2) A.A. is aspartic acid (D), asparagine (N), or serine (S);
(1-2) when No. 4 A.A. is isoleucine (I), each of No. 1 A.A. and No. “ii” (−2) A.A. may be an arbitrary amino acid;
(1-3) when No. 4 A.A. is leucine (L), each of No. 1 A.A. and No. “ii” (−2) A.A. may be an arbitrary amino acid;
(1-4) when No. 4 A.A. is methionine (M), each of No. 1 A.A. and No. “ii” (−2) A.A. may be an arbitrary amino acid;
(1-5) when No. 4 A.A. is asparagine (N), each of No. 1 A.A. and No. “ii” (−2) A.A. may be an arbitrary amino acid;
(1-6) when No. 4 A.A. is proline (P), each of No. 1 A.A. and No. “ii” (−2) A.A. may be an arbitrary amino acid;
(1-7) when No. 4 A.A. is serine (S), each of No. 1 A.A. and No. “ii” (−2) A.A. may be an arbitrary amino acid;
(1-8) when No. 4 A.A. is threonine (T), each of No. 1 A.A. and No. “ii” (−2) A.A. may be an arbitrary amino acid; and
(1-9) when No. 4 A.A. is valine (V), each of No. 1 A.A. and No. “ii” (−2) A.A. may be an arbitrary amino acid.
[17] The method according to [15], wherein the combination of the three amino acids of No. 1 A.A., No. 4 A.A., and No. “ii” (−2) A.A. is determined according to any one of the following definitions:
(2-1) when the three amino acids, No. 1 A.A., No. 4 A.A., and No. “ii” (−2) A.A. are an arbitrary amino acid, glycine, and aspartic acid, respectively, the PPR motif selectively binds to G;
(2-2) when the three amino acids, No. 1 A.A., No. 4 A.A., and No. “ii” (−2) A.A., are glutamic acid, glycine, and aspartic acid, respectively, the PPR motif selectively binds to G;
(2-3) when the three amino acids, No. 1 A.A., No. 4 A.A., and No. “ii” (−2) A.A., are an arbitrary amino acid, glycine, and asparagine, respectively, the PPR motif selectively binds to A;
(2-4) when the three amino acids, No. 1 A.A., No. 4 A.A., and No. “ii” (−2) A.A., are glutamic acid, glycine, and asparagine, respectively, the PPR motif selectively binds to A;
(2-5) when the three amino acids, No. 1 A.A., No. 4 A.A., and No. “ii” (−2) A.A., are an arbitrary amino acid, glycine, and serine, respectively, the PPR motif selectively binds to A, and next binds to C;
(2-6) when the three amino acids, No. 1 A.A., No. 4 A.A., and No. “ii” (−2) A.A., are an arbitrary amino acid, isoleucine, and an arbitrary amino acid, respectively, the PPR motif selectively binds to T and C;
(2-7) when the three amino acids, No. 1 A.A., No. 4 A.A., and No. “ii” (−2) A.A., are an arbitrary amino acid, isoleucine, and asparagine, respectively, the PPR motif selectively binds to T, and next binds to C;
(2-8) when the three amino acids, No. 1 A.A., No. 4 A.A., and No. “ii” (−2) A.A., are an arbitrary amino acid, leucine, and an arbitrary amino acid, respectively, the PPR motif selectively binds to T and C;
(2-9) when the three amino acids, No. 1 A.A., No. 4 A.A., and No. “ii” (−2) A.A., are an arbitrary amino acid, leucine, and aspartic acid, respectively, the PPR motif selectively binds to C;
(2-10) when the three amino acids, No. 1 A.A., No. 4 A.A., and No. “ii” (−2) A.A., are an arbitrary amino acid, leucine, and lysine, respectively, the PPR motif selectively binds to T;
(2-11) when the three amino acids, No. 1 A.A., No. 4 A.A., and No. “ii” (−2) A.A., are an arbitrary amino acid, methionine, and an arbitrary amino acid, respectively, the PPR motif selectively binds to T;
(2-12) when the three amino acids, No. 1 A.A., No. 4 A.A., and No. “ii” (−2) A.A., are an arbitrary amino acid, methionine, and aspartic acid, respectively, the PPR motif selectively binds to T;
(2-13) when the three amino acids, No. 1 A.A., No. 4 A.A., and No. “ii” (−2) A.A., are isoleucine, methionine, and aspartic acid, respectively, the PPR motif selectively binds to T, and next binds to C;
(2-14) when the three amino acids, No. 1 A.A., No. 4 A.A., and No. “ii” (−2) A.A., are an arbitrary amino acid, asparagine, and an arbitrary amino acid, respectively, the PPR motif selectively binds to C and T;
(2-15) when the three amino acids, No. 1 A.A., No. 4 A.A., and No. “ii” (−2) A.A., are an arbitrary amino acid, asparagine, and aspartic acid, respectively, the PPR motif selectively binds to T;
(2-16) when the three amino acids, No. 1 A.A., No. 4 A.A., and No. “ii” (−2) A.A., are phenylalanine, asparagine, and aspartic acid, respectively, the PPR motif selectively binds to T;
(2-17) when the three amino acids, No. 1 A.A., No. 4 A.A., and No. “ii” (−2) A.A., are glycine, asparagine, and aspartic acid, respectively, the PPR motif selectively binds to T;
(2-18) when the three amino acids, No. 1 A.A., No. 4 A.A., and No. “ii” (−2) A.A., are isoleucine, asparagine, and aspartic acid, respectively, the PPR motif selectively binds to T;
(2-19) when the three amino acids, No. 1 A.A., No. 4 A.A., and No. “ii” (−2) A.A., are threonine, asparagine, and aspartic acid, respectively, the PPR motif selectively binds to T;
(2-20) when the three amino acids, No. 1 A.A., No. 4 A.A., and No. “ii” (−2) A.A. are valine, asparagine, and aspartic acid, respectively, the PPR motif selectively binds to T, and next binds to C;
(2-21) when the three amino acids, No. 1 A.A., No. 4 A.A., and No. “ii” (−2) A.A. are tyrosine, asparagine, and aspartic acid, respectively, the PPR motif selectively binds to T, and next binds to C;
(2-22) when the three amino acids, No. 1 A.A., No. 4 A.A., and No. “ii” (−2) A.A., are an arbitrary amino acid, asparagine, and asparagine, respectively, the PPR motif selectively binds to C;
(2-23) when the three amino acids, No. 1 A.A., No. 4 A.A., and No. “ii” (−2) A.A., are isoleucine, asparagine, and asparagine, respectively, the PPR motif selectively binds to C;
(2-24) when the three amino acids, No. 1 A.A., No. 4 A.A., and No. “ii” (−2) A.A., are serine, asparagine, and asparagine, respectively, the PPR motif selectively binds to C;
(2-25) when the three amino acids, No. 1 A.A., No. 4 A.A., and No. “ii” (−2) A.A., are valine, asparagine, and asparagine, respectively, the PPR motif selectively binds to C;
(2-26) when the three amino acids, No. 1 A.A., No. 4 A.A., and No. “ii” (−2) A.A., are an arbitrary amino acid, asparagine, and serine, respectively, the PPR motif selectively binds to C;
(2-27) when the three amino acids, No. 1 A.A., No. 4 A.A., and No. “ii” (−2) A.A., are valine, asparagine, and serine, respectively, the PPR motif selectively binds to C;
(2-28) when the three amino acids, No. 1 A.A., No. 4 A.A., and No. “ii” (−2) A.A., are an arbitrary amino acid, asparagine, and threonine, respectively, the PPR motif selectively binds to C;
(2-29) when the three amino acids, No. 1 A.A., No. 4 A.A., and No. “ii” (−2) A.A., are valine, asparagine, and threonine, respectively, the PPR motif selectively binds to C;
(2-30) when the three amino acids, No. 1 A.A., No. 4 A.A., and No. “ii” (−2) A.A., are an arbitrary amino acid, asparagine, and tryptophan, respectively, the PPR motif selectively binds to C, and next binds to T;
(2-31) when the three amino acids, No. 1 A.A., No. 4 A.A., and No. “ii” (−2) A.A., are isoleucine, asparagine, and tryptophan, respectively, the PPR motif selectively binds to T, and next binds to C;
(2-32) when the three amino acids, No. 1 A.A., No. 4 A.A., and No. “ii” (−2) A.A., are an arbitrary amino acid, proline, and an arbitrary amino acid, respectively, the PPR motif selectively binds to T;
(2-33) when the three amino acids, No. 1 A.A., No. 4 A.A., and No. “ii” (−2) A.A., are an arbitrary amino acid, proline, and aspartic acid, respectively, the PPR motif selectively binds to T;
(2-34) when the three amino acids, No. 1 A.A., No. 4 A.A., and No. “ii” (−2) A.A., are phenylalanine, proline, and aspartic acid, respectively, the PPR motif selectively binds to T;
(2-35) when the three amino acids, No. 1 A.A., No. 4 A.A., and No. “ii” (−2) A.A., are tyrosine, proline, and aspartic acid, respectively, the PPR motif selectively binds to T;
(2-36) when the three amino acids, No. 1 A.A., No. 4 A.A., and No. “ii” (−2) A.A., are an arbitrary amino acid, serine, and an arbitrary amino acid, respectively, the PPR motif selectively binds to A and G;
(2-37) when the three amino acids, No. 1 A.A., No. 4 A.A., and No. “ii” (−2) A.A., are an arbitrary amino acid, serine, and asparagine, respectively, the PPR motif selectively binds to A;
(2-38) when the three amino acids, No. 1 A.A., No. 4 A.A., and No. “ii” (−2) A.A., are phenylalanine, serine, and asparagine, respectively, the PPR motif selectively binds to A;
(2-39) when the three amino acids, No. 1 A.A., No. 4 A.A., and No. “ii” (−2) A.A., are valine, serine, and asparagine, respectively, the PPR motif selectively binds to A;
(2-40) when the three amino acids, No. 1 A.A., No. 4 A.A., and No. “ii” (−2) A.A., are an arbitrary amino acid, threonine, and an arbitrary amino acid, respectively, the PPR motif selectively binds to A and G;
(2-41) when the three amino acids, No. 1 A.A., No. 4 A.A., and No. “ii” (−2) A.A., are an arbitrary amino acid, threonine, and aspartic acid, respectively, the PPR motif selectively binds to G;
(2-42) when the three amino acids, No. 1 A.A., No. 4 A.A., and No. “ii” (−2) A.A., are valine, threonine, and aspartic acid, respectively, the PPR motif selectively binds to G;
(2-43) when the three amino acids, No. 1 A.A., No. 4 A.A., and No. “ii” (−2) A.A., are an arbitrary amino acid, threonine, and asparagine, respectively, the PPR motif selectively binds to A;
(2-44) when the three amino acids, No. 1 A.A., No. 4 A.A., and No. “ii” (−2) A.A., are phenylalanine, threonine, and asparagine, respectively, the PPR motif selectively binds to A;
(2-45) when the three amino acids, No. 1 A.A., No. 4 A.A., and No. “ii” (−2) A.A., are isoleucine, threonine, and asparagine, respectively, the PPR motif selectively binds to A;
(2-46) when the three amino acids, No. 1 A.A., No. 4 A.A., and No. “ii” (−2) A.A., are valine, threonine, and asparagine, respectively, the PPR motif selectively binds to A;
(2-47) when the three amino acids, No. 1 A.A., No. 4 A.A., and No. “ii” (−2) A.A., are an arbitrary amino acid, valine, and an arbitrary amino acid, respectively, the PPR motif binds with A, C, and T, but does not bind to G;
(2-48) when the three amino acids, No. 1 A.A., No. 4 A.A., and No. “ii” (−2) A.A., are isoleucine, valine, and aspartic acid, respectively, the PPR motif selectively binds to C, and next binds to A;
(2-49) when the three amino acids, No. 1 A.A., No. 4 A.A., and No. “ii” (−2) A.A., are an arbitrary amino acid, valine, and glycine, respectively, the PPR motif selectively binds to C; and
(2-50) when the three amino acids, No. 1 A.A., No. 4 A.A., and No. “ii” (−2) A.A., are an arbitrary amino acid, valine, and threonine, respectively, the PPR motif selectively binds to T.
[18] The method according to any one of [15] to [17], wherein at least one selected from the group consisting of the combination of (b) and (c), the combination of (d) and (e), (a), (g), and (h), preferably at least one selected from the group consisting of the combination of (b) and (c), the combination of (d) and (e), (g), and (h), is satisfied.
[19] The method according to [18], wherein the combination of (b) and (c) is satisfied, and at least one selected from the group consisting of the combination of (d) and (e), (a), (g), and (h), preferably at least one selected from the group consisting of the combination of (d) and (e), (g), and (h), is satisfied.
[20] The method according to [19], wherein the combination of (b) and (c), the combination of (d) and (e), (a), and (g), preferably the combination of (b) and (c), the combination of (d) and (e), and (g), are satisfied.
[21] The method according to any one of [15] to [20], wherein the protein contains a plurality of PPR motifs, and the PPR motifs satisfy any of the following (i) to (viii):
(i) at least 40% of No. 7 A.A. consists of isoleucine (I);
(ii) at least 36% of No. 9 A.A. consists of alanine (A);
(iii) at least 37% of No. 10 A.A. consists of tyrosine (Y);
(iv) at least 19% of No. 18 A.A. consists of lysine (K), arginine (R), or histidine (H);
(v) at least 21% of No. 20 A.A. consists of glutamic acid (E) or aspartic acid (D);
(vi) at least 9% of No. 29 A.A. consists of glutamic acid (E) or aspartic acid (D);
(vii) at least 16% of No. 31 A.A. consists of isoleucine (I); and
(viii) at least 15% of No. 32 A.A. consists of lysine (K), arginine (R), or histidine (H), or

the protein contains a plurality of PPR motifs, and has a DNA-binding PPR motif content of 13% or higher.
[22] A method for producing a protein, which comprises designing a protein by the method according to any one of [14] to [21], and producing the designed protein.
[23] A method for producing a complex, which comprises designing a protein by the method according to any one of [14] to [21], and binding a region consisting of the designed protein and a functional region to produce the complex.
[24] A method for editing a genome, which comprises using the complex according to any one of [10] to [13], or

designing a protein by the method according to any one of [14] to [21], binding a region consisting of the designed protein and a functional region to produce a complex, and using the produced complex (implementation in a human individual is excluded).
[25] A method for producing a cell containing a edited genome, which comprises editing a genome by the method according 23, and producing a cell containing the edited genome (implementation in a human individual is excluded).

### Effect of the Invention

According to the present invention, a PPR motif that can binds to a target DNA base, and a protein containing it can be provided. By arranging two or more PPR motifs, a protein that can binds to a target DNA having an arbitrary sequence or length can be provided. A nucleic acid (DNA or RNA) encoding such a protein, and a transformant using such a nucleic acid can also be provided.

According to the present invention, a complex having an activity to bind to a specific nucleic acid sequence and comprising a protein having a specific function (for example, cleavage, transcription, replication, restoration, synthesis, modification, etc. of DNA) can be prepared. With such a complex, genome editing utilizing a function of the functional region such as cleavage, transcription, replication, restoration, synthesis, modification, etc. of a target can be realized. By the genome editing, a cell or organism having a modified genome can be provided.

### BRIEF DESCRIPTION OF THE DRAWINGS

FIG. 1 shows identification of locations of the amino acids characterizing dPPR proteins. The upper part and the middle part show occurrence frequencies of amino acids of the PPR motifs at all the positions in 9 kinds of dPPR molecules and 5 known rPPR molecules, and the lower part shows the results of F test. The F test was used for comparison of the occurrence frequencies at a significance level of 5% (p<0.06). According to the results of the F test, differences were observed in the amino acid frequencies for the residues of No. 7 amino acid (A. A.), No. 9 A.A., No. 10 A.A., No. 18 A.A., No. 20 A.A., No. 29 A.A., No. 31 A.A., No. 32 A.A., and No. ii A.A. However, No. ii A.A. was excluded, since it is a part involved in recognition of a DNA base.

FIG. 2 shows comparison of DNA-binding powers of modified type crPPRs and naturally occurring dPPRs. The DNA binding ability was analyzed by DNA-protein pull-down assay (refer to Example 1). There were obtained results that DNA-binding powers of all the crPPRs and modified type crPPRs in which each dPPR motif-specific amino acid sequence was inserted were higher than those of GUN1, pTAC2, p63, and DG1, which are naturally occurring type dPPR molecules.

FIG. 3 shows comparison of DNA-binding powers of modified type rPPRs and crPPR (7L/31F). The powers were quantified by standardization in which luminescence intensity of each pulled-down protein was divided with luminescence intensity obtained with input 3%. As a result of the comparison of the DNA-binding powers of the modified type rPPRs and crPPR (7L/31F), significant differences were observed for modified type rPPRs introduced with of A.A. 9A, A.A. 18K, A.A. 31I, A.A. 32K, and A.A. 9A/10Y. The vertical axis indicates DNA-binding power (pull down signal/input 3% signal), the introduced amino acid sequences are mentioned under the horizontal axis, * means p<0.05, and ** means p<0.01.

FIG. 4 shows comparison of the DNA-binding powers observed with replacing amino acids with those having similar characteristics. It was examined whether the effect can be obtained even when amino acids having similar characteristics are used for A.A. 18K, A.A. 31I, A.A. 32K, and A.A. 9A/10Y. In this experiment, there were introduced histidine (H) and arginine (R), which are basic amino acids like K, for No. 18 A.A. and No. 32 A.A., valine (V) and leucine (L), which have a branched chain like I, for No. 31 A.A., and phenylalanine (F) and tryptophan (W), which have an aromatic group like Y, for No. 10 A.A. As a result of comparison of the DNA-binding powers of the modified type rPPRs and crPPR (7L/31F), significant differences were observed for all the modified type rPPRs. The vertical axis indicates DNA-binding ability (pull down signal/input 3% signal), the introduced amino acid sequences are mentioned under the horizontal axis, * means p<0.05, and ** means p<0.01.

FIG. 5 shows comparison of the DNA-binding powers of the proteins having different contents of DNA-binding PPR motifs. In this experiment, there were analyzed DNA-binding powers of modified type rPPRs consisting of crPPR (7L/31F) in which 2 motifs (25% of the whole) or 4 motifs (50% of the whole) from the N-terminus were motifs having these amino acid sequences. Significant differences were observed for all the modified type rPPRs. The vertical axis indicates DNA-binding power (pull down signal/input 3% signal), the introduced amino acid sequences and contents thereof are mentioned under the horizontal axis, * means p<0.05, and ** means p<0.01.

FIG. 6 shows comparison of the DNA-binding powers of naturally occurring type dPPR proteins and modified type PPR proteins thereof. It was examined whether the DNA-binding ability of modified proteins of naturally occurring type dPPRs, P63 and GUN1, in which A.A. 9A/10Y/18K/31I, and A.A. 31I/32K were introduced into all the motifs thereof. The DNA-binding powers of all the P63 and GUN1 proteins introduced with any of the amino acid sequences were increased. The vertical axis indicates DNA-binding power (pull down signal/input 3% signal) calculated as relative value based on those of naturally occurring type dPPR proteins, the types of dPPR are mentioned under the horizontal axis, * means p<0.05, and ** means p<0.01.

### MODES FOR CARRYING OUT THE INVENTION

### [PPR Motif and PPR Protein]

The “PPR motif” referred to in the present invention means a polypeptide constituted with 30 to 38 amino acids and having an amino acid sequence that shows, when the amino acid sequence is analyzed with a protein domain search program on the web (for example, Pfam, Prosite, Uniprot, etc.), an E value not larger than a predetermined value (desirably E-03) obtained at PF01535 in the case of Pfam (http://pfam.sanger.ac.uk/), or PS51375 in the case of Prosite (http://www.expasy.org/prosite/), unless otherwise indicated. The PPR motifs in various proteins are also defined in the Uniprot database (http://www.uniprot.org).

Although the amino acid sequence of the PPR motif is not highly conserved in the PPR motif of the present invention, such a secondary structure of helix, loop, helix, and loop as shown by the following formula is conserved well.

[Chemical Formula 3]

(Helix A)-X-(Helix B)-L (Formula 1)

The position numbers of the amino acids constituting the PPR motif defined in the present invention are according to those defined in a paper of the inventors of the present invention (Kobayashi K, et al., Nucleic Acids Res., 40, 2712-2723 (2012)), and Patent document 4, unless especially indicated. That is, the position numbers of the amino acids constituting the PPR motif defined in the present invention are substantially the same as the amino acid numbers defined for PF01535 in Pfam, but correspond to numbers obtained by subtracting 2 from the amino acid numbers defined for PS51375 in Prosite (for example, position 1 according to the present invention is position 3 of PS51375), and also correspond to numbers obtained by subtracting 2 from the amino acid numbers of the PPR motif defined in Uniprot.

More precisely, in the present invention, the No. 1 amino acid is the first amino acid from which Helix A shown in the formula 1 starts. The No. 4 amino acid is the fourth amino acid counted from the No. 1 amino acid. As for “ii” (−2)nd amino acid,

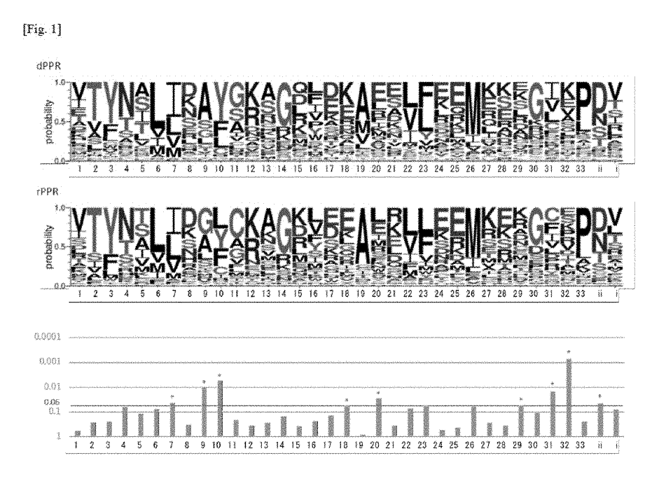

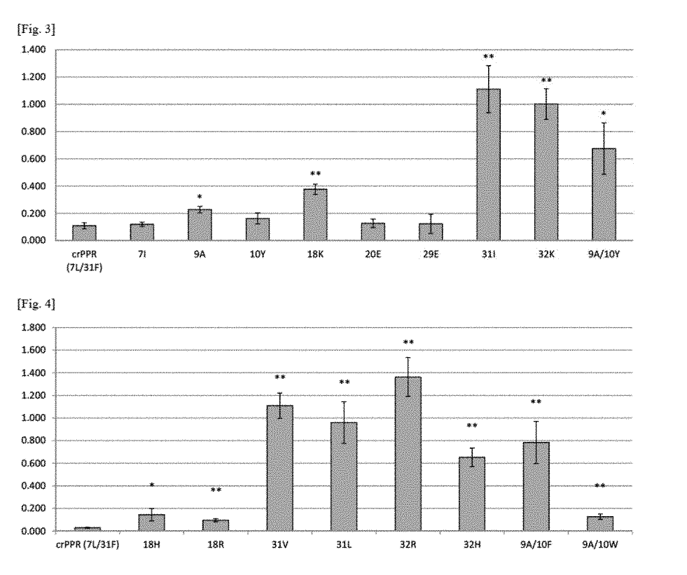

when a next PPR motif (Mₙ₊₁) contiguously exists on the C-terminus side of the PPR motif (Mₙ) (when there is no amino acid insertion between the PPR motifs, as in the cases of, for example, Motif Nos. 1, 2, 3,4, 6 and 7 in FIG. 4-1 (A) of Patent document 4), the −2nd amino acid counted from the end (C-terminus side) of the amino acids constituting the PPR motif (Mₙ) is referred to as No. “ii” (−2) amino acid;
when a non-PPR motif (part that is not the PPR motif) consisting of 1 to 20 amino acids exists between the PPR motif (Mₙ) and the next PPR motif (Mₙ₊₁) on the C-terminus side (as in the cases of, for example, Motif Nos. 5 and 8 in FIG. 4-1 (A) of Patent document 4, and Motif Nos. 1, 2, 7 and 8 in FIG. 4-3 (D) of Patent document 4), the amino acid locating upstream of the first amino acid of the next PPR motif (Mₙ₊₁) by 2 positions, i.e., the −2nd amino acid, is referred to as No. “ii” (−2) amino acid (refer to FIG. 1 of Patent document 4); or
when any next PPR motif (Mₙ₊₁) does not exist on the C-terminus side of the PPR motif (Mₙ) (as in the cases of, for example, Motif No. 9 in FIG. 4-1 (A) of Patent document 4, and Motif No. 11 in FIG. 4-1 (B) of Patent document 4), or 21 or more amino acids constituting a non-PPR motif exist between the PPR motif (Mₙ) and the next PPR motif (Mₙ₊₁) on the C-terminus side, the 2nd amino acid counted from the end (C-terminus side) of the amino acids constituting the PPR motif (Mₙ) is referred to as No. “ii” (−2) amino acid.

The positions of No. 31 A.A. and No. 32 A.A., which are amino acids contained in L of a certain PPR motif (Mₙ), may be determined on the basis of No. 1 amino acid of the next PPR motif (Mₙ₊₁) on the C-terminus side of that motif. Specifically, the No. 31 A.A. may be determined to be an amino acid locating upstream from the No. 1 amino acid of the next PPR motif (Mₙ₊₁) by 5 amino acids, and the No. 32 A.A. may be determined to be an amino acid locating upstream from the No. 1 amino acid of the next PPR motif (Mₙ₊₁) by 4 amino acids. When the next PPR motif (Mₙ₊₁) does not exist on the C-terminus side of the PPR motif (Mₙ), the 5th amino acid from the last amino acid (C-terminus side) among the amino acids constituting the PPR motif (Mₙ) is determined to be No. 31 A.A., and the amino acid locating upstream from the same by 4 amino acids is determined to be No. 32 A.A.

The “PPR protein” or “PPR molecule” referred to in the present invention means a PPR protein having one or more of the aforementioned PPR motifs, unless otherwise indicated. The term “protein” used in this specification means any substance consisting of a polypeptide (chain consisting of two or more amino acids bound through peptide bonds), and also includes those consisting of a comparatively low molecular weight polypeptide, unless otherwise indicated. The “amino acid” referred to in the present invention means a usual amino acid molecule, as well as an amino acid residue constituting a peptide chain. Which the term means will be apparent to those skilled in the art from the context.

Many PPR proteins exist in plants, and 500 proteins and about 5000 motifs can be found in Arabidopsis thaliana. PPR motifs and PPR proteins of various amino acid sequences also exist in many land plants such as rice, poplar, and selaginella. It is known that some PPR proteins are important factors for obtaining Fl seeds for hybrid vigor as fertility restoration factors that are involved in formation of pollen (male gamete). It has been clarified that some PPR proteins are involved in speciation, similarly in fertility restoration. It has also been clarified that almost all the PPR proteins act on RNA in mitochondria or chloroplasts.

It is known that, in animals, anomaly of the PPR protein identified as LRPPRC causes Leigh syndrome French Canadian (LSFC, Leigh's syndrome, subacute necrotizing encephalomyelopathy).

The term “selective” used for a property of a PPR motif for binding with a DNA base in the present invention means that a binding activity for any one base among the DNA bases is higher than binding activities for the other bases, unless otherwise indicated. Those skilled in the art can confirm this selectivity by planning an experiment, or it can also be obtained by calculation as described in the examples mentioned in Patent document 4.

The DNA base referred to in the present invention means a base of deoxyribonucleotide constituting DNA, and specifically, it means any of adenine (A), guanine (G), cytosine (C), and thymine (T), unless otherwise indicated. Although the PPR protein may have selectivity to a base in DNA, it does not bind to a nucleic acid monomer.

[Information, Novel dPPR Protein, Etc. Provided by the Present Invention]

The present invention provides information about positions and types of amino acids important for binding with DNA, a method for designing a dPPR protein, a method for imparting a property of binding with a DNA base to a PPR protein, and a method for enhancing a property of a PPR protein for binding with DNA, which methods use the information, as well as a novel dPPR protein obtained by the aforementioned designing method, method for imparting the binding property, or method for enhancing the binding property. The origins of the dPPR protein provided by the present invention and the dPPR protein used in the present invention, and the methods for obtaining them are not particularly limited, and they may be, for example, naturally occurring dPPRs, modified naturally occurring dPPRs, dPPRs obtained by chemical synthesis, recombinant proteins of the foregoing, or the like, and they may also be fused proteins. Various dPPR proteins and embodiments using them fall within the scope of the present invention so long as they satisfy the requirements defined in the appended claims.

Designing a protein may be determining amino acid sequence of a protein according to the information provided by the present invention. Designing a protein may also be, in other words, producing a protein. The method for designing a protein, or the method for producing a protein includes the following steps:

the step of determining nucleotide sequence encoding a protein;

the step of preparing a polynucleotide having the nucleotide sequence; and

the step of preparing a transformant that is introduced with the polynucleotide, and can produce the protein.

The information about the positions of amino acids of PPR proteins important for base-selective or sequence-specific binding is disclosed in Patent documents 3 and 4. Further, according to the investigations of the inventors of the present invention, in addition to the aforementioned information, No. 7 amino acid (A.A.), No. 9 A.A., No. 10 A.A., No. 18 A.A., No. 20 A.A., No. 29 A.A., No. 31 A.A., No. 32 A.A., and No. ii A.A., preferably No. 9 A.A., No. 10 A.A., No. 18 A.A., No. 20 A.A., No. 29 A.A., No. 31 A.A., No. 32 A.A. and No. ii A.A., of the PPR motif (Mₙ) are important for binding with DNA. By paying attention to these, a property of binding with a DNA base can be imparted to PPR proteins, or a property of binding with DNA of PPR proteins can be enhanced. Since No. ii A.A. is a part involved in recognition of a DNA base, it may be excluded.

Whether a certain PPR protein has a property of binding with DNA, or degree of the binding ability of a certain PPR protein can be appropriately evaluated by those skilled in the art by planning an appropriate DNA-protein pull-down assay, or the like. As for specific experimental conditions and procedures, the sections of Examples of Patent document 4 and this specification can be referred to.

The ability of binding with DNA of the PPR protein obtained by the present invention is higher than the same of the modified PPR consisting of the consensus PPR (cPPR, also referred to as crPPR) reported in Non-patent document 15 (Coquille et al., 2014, An artificial PPR scaffold for programmable RNA recognition) cited below, of which A.A. 71 and A.A. 31I are replaced with leucine (L) and phenylalanine (F), respectively (crPPR (7L/31F)).

The ability of binding with DNA of the PPR protein obtained by the present invention is preferably higher than the same of existing DNA-binding PPRs, specifically, any one among the group consisting of p63 (SEQ ID NO: 1), GUN1 (SEQ ID NO: 2), pTac2 (SEQ ID NO: 3), DG1 (SEQ ID NO: 4), and GRP23 (SEQ ID NO: 5), more preferably higher than the abilities of binding with DNA of all of these proteins. The protein more preferably selectively binds with DNA among RNA and DNA having substantially the same sequences.

Impartation of a property of binding with DNA to a PPR protein and enhancement of a property of binding with DNA of a PPR protein can be achieved by, specifically, designing the PPR motif (Mₙ) of a base-selectively or base sequence-specifically bindable PPR protein so that it satisfies at least one condition selected from the group consisting of (a) to (h), preferably (b) to (h), mentioned below:
(a) No. 7 A.A. of the PPR motif (Mₙ) is isoleucine (I);
(b) No. 9 A.A. of the PPR motif (Mₙ) is alanine (A);
(c) No. 10 A.A. of the PPR motif (Mₙ) is tyrosine (Y), phenylalanine (F), or tryptophan (W);
(d) No. 18 A.A. of the PPR motif (Mₙ) is lysine (K), arginine (R), or histidine (H);
(e) No. 20 A.A. of the PPR motif (Mₙ) is glutamic acid (E), or aspartic acid (D);
(f) No. 29 A.A. of the PPR motif (Mₙ) is glutamic acid (E), or aspartic acid (D).
(g) No. 31 A.A. of the PPR motif (Mₙ) is isoleucine (I), leucine (L), or valine (V); and
(h) No. 32 A.A. of the PPR motif (Mₙ) is lysine (K), arginine (R), or histidine (H)

According to the investigations of the inventors of the present invention, when a DNA-binding ability of a certain PPR can be enhanced by using a specific amino acid at an appropriate position, the same effect can be obtained even if an amino acid having similar characteristics is used instead of the specific amino acid. It can be said that the amino acids of the following sets have similar characteristics: glycine and alanine (these have an alkyl chain), valine, leucine, and isoleucine (these have a branched alkyl chain), phenylalanine, tyrosine, and tryptophan (these have an aromatic group), lysine, arginine, and histidine (these have two amino groups, and are basic), aspartic acid and glutamic acid (these have two carboxyl groups and are acidic), asparagine and glutamine (these have amide group), serine and threonine (these have hydroxyl group), and cysteine and methionine (these contain sulfur).

According to the investigations of the inventors of the present invention, there are a tendency that A as No. 9 A.A. and Y as No. 10 A.A. are observed in the same motif, and a tendency that, when No. 18 A.A. is K, R, or H, No. 20 A.A. of the preceding motif is E or D. From this point of view, in one of preferred embodiments, the PPR motif (Mₙ) satisfies at least one selected from the group consisting of the combination of (b) and (c), the combination of (d) and (e), (a), (g), and (h), more preferably at least one selected from the group consisting of the combination of (b) and (c), the combination of (d) and (e), (g), and (h). In another preferred embodiment, the PPR motif (Mₙ) satisfies the combination of (b) and (c), and at least one selected from the group consisting of the combination of (d) and (e), (a), (g), and (h), more preferably the PPR motif (Mₙ) satisfies the combination of (b) and (c), and satisfies at least one selected from the group consisting of the combination of (d) and (e), (g), and (h). In still another preferred embodiment, the PPR motif (Mₙ) satisfies the combination of (b) and (c), the combination of (d) and (e), (a), and (g), more preferably the combination of (b) and (c), the combination of (d) and (e), and (g).

The PPR protein to be designed contains one or more PPR motifs (Mₙ), and it preferably contains 2 to 30, more preferably 5 to 25, still more preferably 9 to 15, of the motifs.

In the case of the protein containing two or more PPR motifs, if it is designed so that a certain part of the motifs satisfy the aforementioned conditions, a property of binding with a DNA base can be imparted to the PPR protein, or a property of binding with DNA of the PPR protein can be enhanced, even if all the contained motifs do not satisfy the requirements. For example, the protein containing two or more PPR motifs that satisfy any one of (i) to (viii) mentioned below (for example, any one, preferably any three, more preferably any five, further preferably all of them) constitutes one of the preferred embodiments of the present invention:
(i) at least 40%, preferably 44%, of No. 7 A.A. consists of isoleucine (I);
(ii) at least 36%, preferably 48%, of No. 9 A.A. consists of alanine (A);
(iii) at least 37%, preferably 49%, of No. 10 A.A. consists of tyrosine (Y);
(iv) at least 19% of No. 18 A.A. consists of lysine (K), arginine (R), or histidine (H);
(v) at least 21% of No. 20 A.A. consists of glutamic acid (E) or aspartic acid (D);
(vi) at least 9% of No. 29 A.A. consists of glutamic acid (E) or aspartic acid (D);
(vii) at least 16% of No. 31 A.A. consists of isoleucine (I); and
(viii) at least 15% of No. 32 A.A. is lysine (K), arginine (R), or histidine (H).

The ratios (%) mentioned above are calculated as [number of PPR motifs satisfying requirement]/[total number of PPR motifs contained in protein]×100.

The PPR motif satisfying requirement is a DNA-binding PPR motif, and it refers to a PPR motif that satisfies at least one selected from the group consisting (b) to (h) mentioned above. More specifically, the ratio of DNA-binding PPR motif mentioned above may be referred to as “content of DNA-binding PPR motif”, and calculated as [number of DNA-binding PPR motifs]/[(number of DNA-binding PPR motifs)+(number of PPR motifs that are not DNA-binding PPR motifs)]×100. The PPR motif that is not a DNA-binding PPR motif refers to a PPR motif that does not satisfy all of (b) to (h) mentioned above, for example, crPPR (7L/31F).

According to the further investigations of the inventors of the present invention, in the case of a protein containing 8 PPR motifs, the DNA-binding ability thereof was significantly increased when it had a DNA-binding PPR motif content of 25% or higher, compared with a control protein of which DNA-binding PPR motif content is 0%, whereas significant increase of the DNA-binding ability was not observed for the protein of which DNA-binding PPR motif content was 12.5% compared with the control protein of which DNA-binding PPR motif content is 0%. Therefore, the PPR protein preferably contains two or more PPR motifs, and has a DNA-binding PPR motif content of 13% or higher, more preferably 15% or higher, further preferably 25% or higher, still further preferably 50% or higher, still further preferably 75% or more, still further preferably 100%.

Although the positions of DNA-binding PPRs in the protein containing two or more PPR motifs are not particularly limited, positions closer to the N-terminus are preferred. When the protein contains two or more PPR motifs, and the PPR motifs consist of two or more DNA-binding PPR motifs and PPR motifs that are not DNA-binding PPR motif, the DNA-binding PPR motifs may contiguously exist, or a PPR motif that is not DNA-binding PPR motif may exist between the DNA-binding PPR motifs, but it is considered that the DNA-binding PPR motifs preferably contiguously exist. For example, it is considered that, in the case of the protein containing 8 PPR motifs, it is preferred that 2 contiguous PPR motifs on the N-terminus side are DNA-binding PPR motifs, when the DNA-binding PPR motif content is 25%, it is preferred that 4 contiguous PPR motifs on the N-terminus side are DNA-binding PPR motifs, when the DNA-binding PPR motif content is 50%, and it is preferred that 6 contiguous PPR motifs on the N-terminus side are DNA-binding PPR motifs, when the DNA-binding PPR motif content is 75%.

The aforementioned method for imparting a property of binding with DNA to a PPR protein, or enhancing a property of binding with DNA of a PPR protein can be used not only for newly designing a DNA-binding PPR protein, but also for imparting a DNA-binding ability to an existing PPR protein, or increasing DNA-binding ability of an existing PPR protein.

The information about the positions and types of amino acids of PPR protein important for base-selective or sequence-specific binding described in Patent documents 3 and 4, which serves as the basis of the designing method of the present invention for imparting a property of binding with a DNA base to a PPR protein, or enhancing a property of binding with DNA of a PPR protein, is shown below.
(1-1) When No. 4 A.A. is glycine (G), No. 1 A.A. may be an arbitrary amino acid, No. “ii” (−2) A.A. is aspartic acid (D), asparagine (N), or serine (S), and the combination of No. 1 A.A., and No. “ii” (−2) A.A. may be, for example:
a combination of an arbitrary amino acid and aspartic acid (D) (*GD),
preferably a combination of glutamic acid (E) and aspartic acid (D) (EGD),
a combination of an arbitrary amino acid and asparagine (N) (*GN),
preferably a combination of glutamic acid (E) and asparagine (N) (EGN), or
a combination of an arbitrary amino acid and serine (S) (*GS);
(1-2) when No. 4 A.A. is isoleucine (I), each of No. 1 A.A. and No. “ii” (−2) A.A. may be an arbitrary amino acid, and the combination of No. 1 A.A., and No. “ii” (−2) A.A. may be, for example:
a combination of an arbitrary amino acid and asparagine (N) (*IN);
(1-3) when No. 4 A.A. is leucine (L), each of No. 1 A.A. and No. “ii” (−2) A.A. may be an arbitrary amino acid, and the combination of No. 1 A.A., and No. “ii” (−2) A.A. may be, for example:
a combination of an arbitrary amino acid and aspartic acid (D) (*LD), or
a combination of an arbitrary amino acid and lysine (K) (*LK);
(1-4) when No. 4 A.A. is methionine (M), each of No. 1 A.A. and No. “ii” (−2) A.A. may be an arbitrary amino acid, and the combination of No. 1 A.A., and No. “ii” (−2) A.A. may be, for example:
a combination of an arbitrary amino acid and aspartic acid (D) (*MD), or
a combination of isoleucine (I) and aspartic acid (D) (IMD);
(1-5) when No. 4 A.A. is asparagine (N), each of No. 1 A.A. and No. “ii” (−2) A.A. may be an arbitrary amino acid, and the combination of No. 1 A.A., and No. “ii” (−2) A.A. may be, for example:
a combination of an arbitrary amino acid and aspartic acid (D) (*ND),
a combination of any one of phenylalanine (F), glycine (G), isoleucine (I), threonine (T), valine (V) and tyrosines (Y), and aspartic acid (D) (FND, GND, IND, TND, VND, or YND),
a combination of an arbitrary amino acid and asparagine (N) (*NN),
a combination of any one of isoleucine (I), serine (S) and valine (V), and asparagine (N) (INN, SNN or VNN)
a combination of an arbitrary amino acid and serine (S) (*NS),
a combination of valine (V) and serine (S) (VNS),
a combination of an arbitrary amino acid and threonine (T) (*NT),
a combination of valine (V) and threonine (T) (VNT),
a combination of an arbitrary amino acid and tryptophan (W) (*NW), or
a combination of isoleucine (I) and tryptophan (W) (INW);
(1-6) when No. 4 A.A. is proline (P), each of No. 1 A.A. and No. “ii” (−2) A.A. may be an arbitrary amino acid, and the combination of No. 1 A.A., and No. “ii” (−2) A.A. may be, for example:
a combination of an arbitrary amino acid and aspartic acid (D) (*PD),
a combination of phenylalanine (F) and aspartic acid (D) (FPD), or
a combination of tyrosine (Y) and aspartic acid (D) (YPD);
(1-7) when No. 4 A.A. is serine (S), each of No. 1 A.A. and No. “ii” (−2) A.A. may be an arbitrary amino acid, and the combination of No. 1 A.A., and No. “ii” (−2) A.A. may be, for example:
a combination of an arbitrary amino acid and asparagine (N) (*SN),
a combination of phenylalanine (F) and asparagine (N) (FSN), or
a combination of valine (V) and asparagine (N) (VSN);
(1-8) when No. 4 A.A. is threonine (T), each of No. 1 A.A. and No. “ii” (−2) A.A. may be an arbitrary amino acid, and the combination of No. 1 A.A., and No. “ii” (−2) A.A. may be, for example:
a combination of an arbitrary amino acid and aspartic acid (D) (*TD),
a combination of valine (V) and aspartic acid (D) (VTD),
a combination of an arbitrary amino acid and asparagine (N) (*TN),
a combination of phenylalanine (F) and asparagine (N) (FTN),
a combination of isoleucine (I) and asparagine (N) (ITN), or
a combination of valine (V) and asparagine (N) (VTN); and
(1-9) when No. 4 A.A. is valine (V), each of No. 1 A.A. and No. “ii” (−2) A.A. may be an arbitrary amino acid, and the combination of No. 1 A.A., and No. “ii” (−2) A.A. may be, for example:
a combination of isoleucine (I) and aspartic acid (D) (IVD),
a combination of an arbitrary amino acid and glycine (G) (*VG), or
a combination of an arbitrary amino acid and threonine (T) (*VT).

More detailed information about the positions and types of amino acids important for base-selective or sequence-specific binding is shown below. The following explanations are made for DNA base-selective or DNA sequence-specific binding as examples, but those skilled in the art can understand that they can also appropriately apply to RNA base and RNA sequence.

The protein is a protein determined on the basis of the following definitions, and having a selective DNA base-binding property:
(2-1) when the three amino acids, No. 1 A.A., No. 4 A.A., and No. “ii” (−2) A.A., are an arbitrary amino acid, glycine, and aspartic acid, respectively, the PPR motif selectively binds to G;
(2-2) when the three amino acids, No. 1 A.A., No. 4 A.A., and No. “ii” (−2) A.A., are glutamic acid, glycine, and aspartic acid, respectively, the PPR motif selectively binds to G;
(2-3) when the three amino acids, No. 1 A.A., No. 4 A.A., and No. “ii” (−2) A.A., are an arbitrary amino acid, glycine, and asparagine, respectively, the PPR motif selectively binds to A;
(2-4) when the three amino acids, No. 1 A.A., No. 4 A.A., and No. “ii” (−2) A.A., are glutamic acid, glycine, and asparagine, respectively, the PPR motif selectively binds to A;
(2-5) when the three amino acids, No. 1 A.A., No. 4 A.A., and No. “ii” (−2) A.A., are an arbitrary amino acid, glycine, and serine, respectively, the PPR motif selectively binds to A, and next binds to C;
(2-6) when the three amino acids, No. 1 A.A., No. 4 A.A., and No. “ii” (−2) A.A., are an arbitrary amino acid, isoleucine, and an arbitrary amino acid, respectively, the PPR motif selectively binds to T and C;
(2-7) when the three amino acids, No. 1 A.A., No. 4 A.A., and No. “ii” (−2) A.A., are an arbitrary amino acid, isoleucine, and asparagine, respectively, the PPR motif selectively binds to T, and next binds to C;
(2-8) when the three amino acids, No. 1 A.A., No. 4 A.A., and No. “ii” (−2) A.A., are an arbitrary amino acid, leucine, and an arbitrary amino acid, respectively, the PPR motif selectively binds to T and C;
(2-9) when the three amino acids, No. 1 A.A., No. 4 A.A., and No. “ii” (−2) A.A., are an arbitrary amino acid, leucine, and aspartic acid, respectively, the PPR motif selectively binds to C;
(2-10) when the three amino acids, No. 1 A.A., No. 4 A.A., and No. “ii” (−2) A.A., are an arbitrary amino acid, leucine, and lysine, respectively, the PPR motif selectively binds to T;
(2-11) when the three amino acids, No. 1 A.A., No. 4 A.A., and No. “ii” (−2) A.A., are an arbitrary amino acid, methionine, and an arbitrary amino acid, respectively, the PPR motif selectively binds to T;
(2-12) when the three amino acids, No. 1 A.A., No. 4 A.A., and No. “ii” (−2) A.A., are an arbitrary amino acid, methionine, and aspartic acid, respectively, the PPR motif selectively binds to T;
(2-13) when the three amino acids, No. 1 A.A., No. 4 A.A., and No. “ii” (−2) A.A., are isoleucine, methionine, and aspartic acid, respectively, the PPR motif selectively binds to T, and next binds to C;
(2-14) when the three amino acids, No. 1 A.A., No. 4 A.A., and No. “ii” (−2) A.A., are an arbitrary amino acid, asparagine, and an arbitrary amino acid, respectively, the PPR motif selectively binds to C and T;
(2-15) when the three amino acids, No. 1 A.A., No. 4 A.A., and No. “ii” (−2) A.A., are an arbitrary amino acid, asparagine, and aspartic acid, respectively, the PPR motif selectively binds to T;
(2-16) when the three amino acids, No. 1 A.A., No. 4 A.A., and No. “ii” (−2) A.A., are phenylalanine, asparagine, and aspartic acid, respectively, the PPR motif selectively binds to T;
(2-17) when the three amino acids, No. 1 A.A., No. 4 A.A., and No. “ii” (−2) A.A., are glycine, asparagine, and aspartic acid, respectively, the PPR motif selectively binds to T;
(2-18) when the three amino acids, No. 1 A.A., No. 4 A.A., and No. “ii” (−2) A.A., are isoleucine, asparagine, and aspartic acid, respectively, the PPR motif selectively binds to T;
(2-19) when the three amino acids, No. 1 A.A., No. 4 A.A., and No. “ii” (−2) A.A., are threonine, asparagine, and aspartic acid, respectively, the PPR motif selectively binds to T;
(2-20) when the three amino acids, No. 1 A.A., No. 4 A.A., and No. “ii” (−2) A.A. are valine, asparagine, and aspartic acid, respectively, the PPR motif selectively binds to T, and next binds to C;
(2-21) when the three amino acids, No. 1 A.A., No. 4 A.A., and No. “ii” (−2) A.A. are tyrosine, asparagine, and aspartic acid, respectively, the PPR motif selectively binds to T, and next binds to C;
(2-22) when the three amino acids, No. 1 A.A., No. 4 A.A., and No. “ii” (−2) A.A., are an arbitrary amino acid, asparagine, and asparagine, respectively, the PPR motif selectively binds to C;
(2-23) when the three amino acids, No. 1 A.A., No. 4 A.A., and No. “ii” (−2) A.A., are isoleucine, asparagine, and asparagine, respectively, the PPR motif selectively binds to C;
(2-24) when the three amino acids, No. 1 A.A., No. 4 A.A., and No. “ii” (−2) A.A., are serine, asparagine, and asparagine, respectively, the PPR motif selectively binds to C;
(2-25) when the three amino acids, No. 1 A.A., No. 4 A.A., and No. “ii” (−2) A.A., are valine, asparagine, and asparagine, respectively, the PPR motif selectively binds to C;
(2-26) when the three amino acids, No. 1 A.A., No. 4 A.A., and No. “ii” (−2) A.A., are an arbitrary amino acid, asparagine, and serine, respectively, the PPR motif selectively binds to C;
(2-27) when the three amino acids, No. 1 A.A., No. 4 A.A., and No. “ii” (−2) A.A., are valine, asparagine, and serine, respectively, the PPR motif selectively binds to C;
(2-28) when the three amino acids, No. 1 A.A., No. 4 A.A., and No. “ii” (−2) A.A., are an arbitrary amino acid, asparagine, and threonine, respectively, the PPR motif selectively binds to C;
(2-29) when the three amino acids, No. 1 A.A., No. 4 A.A., and No. “ii” (−2) A.A., are valine, asparagine, and threonine, respectively, the PPR motif selectively binds to C;
(2-30) when the three amino acids, No. 1 A.A., No. 4 A.A., and No. “ii” (−2) A.A., are an arbitrary amino acid, asparagine, and tryptophan, respectively, the PPR motif selectively binds to C, and next binds to T;
(2-31) when the three amino acids, No. 1 A.A., No. 4 A.A., and No. “ii” (−2) A.A., are isoleucine, asparagine, and tryptophan, respectively, the PPR motif selectively binds to T, and next binds to C;
(2-32) when the three amino acids, No. 1 A.A., No. 4 A.A., and No. “ii” (−2) A.A., are an arbitrary amino acid, proline, and an arbitrary amino acid, respectively, the PPR motif selectively binds to T;
(2-33) when the three amino acids, No. 1 A.A., No. 4 A.A., and No. “ii” (−2) A.A., are an arbitrary amino acid, proline, and aspartic acid, respectively, the PPR motif selectively binds to T;
(2-34) when the three amino acids, No. 1 A.A., No. 4 A.A., and No. “ii” (−2) A.A., are phenylalanine, proline, and aspartic acid, respectively, the PPR motif selectively binds to T;
(2-35) when the three amino acids, No. 1 A.A., No. 4 A.A., and No. “ii” (−2) A.A., are tyrosine, proline, and aspartic acid, respectively, the PPR motif selectively binds to T;
(2-36) when the three amino acids, No. 1 A.A., No. 4 A.A., and No. “ii” (−2) A.A., are an arbitrary amino acid, serine, and an arbitrary amino acid, respectively, the PPR motif selectively binds to A and G;
(2-37) when the three amino acids, No. 1 A.A., No. 4 A.A., and No. “ii” (−2) A.A., are an arbitrary amino acid, serine, and asparagine, respectively, the PPR motif selectively binds to A;
(2-38) when the three amino acids, No. 1 A.A., No. 4 A.A., and No. “ii” (−2) A.A., are phenylalanine, serine, and asparagine, respectively, the PPR motif selectively binds to A;
(2-39) when the three amino acids, No. 1 A.A., No. 4 A.A., and No. “ii” (−2) A.A., are valine, serine, and asparagine, respectively, the PPR motif selectively binds to A;
(2-40) when the three amino acids, No. 1 A.A., No. 4 A.A., and No. “ii” (−2) A.A., are an arbitrary amino acid, threonine, and an arbitrary amino acid, respectively, the PPR motif selectively binds to A and G;
(2-41) when the three amino acids, No. 1 A.A., No. 4 A.A., and No. “ii” (−2) A.A., are an arbitrary amino acid, threonine, and aspartic acid, respectively, the PPR motif selectively binds to G;
(2-42) when the three amino acids, No. 1 A.A., No. 4 A.A., and No. “ii” (−2) A.A., are valine, threonine, and aspartic acid, respectively, the PPR motif selectively binds to G;
(2-43) when the three amino acids, No. 1 A.A., No. 4 A.A., and No. “ii” (−2) A.A., are an arbitrary amino acid, threonine, and asparagine, respectively, the PPR motif selectively binds to A;
(2-44) when the three amino acids, No. 1 A.A., No. 4 A.A., and No. “ii” (−2) A.A., are phenylalanine, threonine, and asparagine, respectively, the PPR motif selectively binds to A;
(2-45) when the three amino acids, No. 1 A.A., No. 4 A.A., and No. “ii” (−2) A.A., are isoleucine, threonine, and asparagine, respectively, the PPR motif selectively binds to A;
(2-46) when the three amino acids, No. 1 A.A., No. 4 A.A., and No. “ii” (−2) A.A., are valine, threonine, and asparagine, respectively, the PPR motif selectively binds to A;
(2-47) when the three amino acids, No. 1 A.A., No. 4 A.A., and No. “ii” (−2) A.A., are an arbitrary amino acid, valine, and an arbitrary amino acid, respectively, the PPR motif binds with A, C, and T, but does not bind to G;
(2-48) when the three amino acids, No. 1 A.A., No. 4 A.A., and No. “ii” (−2) A.A., are isoleucine, valine, and aspartic acid, respectively, the PPR motif selectively binds to C, and next binds to A;
(2-49) when the three amino acids, No. 1 A.A., No. 4 A.A., and No. “ii” (−2) A.A., are an arbitrary amino acid, valine, and glycine, respectively, the PPR motif selectively binds to C; and
(2-50) when the three amino acids, No. 1 A.A., No. 4 A.A., and No. “ii” (−2) A.A., are an arbitrary amino acid, valine, and threonine, respectively, the PPR motif selectively binds to T.

In the designing for base-selective or sequence-specific binding, amino acids other than those of the combination of the amino acids of No. 1 A.A., No. 4 A.A., and No. “ii” (−2) A.A. may be taken into consideration. For example, selection of the amino acids of No. 8 and No. 12 described in Patent document 2 mentioned above may be important for exhibiting a DNA-binding activity. According to the researches of the inventors of the present invention, the No. 8 amino acid of a certain PPR motif and the No. 12 amino acid of the same PPR motif may cooperate in binding with DNA. The No. 8 amino acid may be a basic amino acid, preferably lysine, or an acidic amino acid, preferably aspartic acid, and the No. 12 amino acid may be a basic amino acid, neutral amino acid, or hydrophobic amino acid.

When a target protein is designed, sequence information of the naturally occurring type PPR motifs of such DNA-binding PPR proteins as mentioned as SEQ ID NOS: 1 to 5, or crPPR motif shown as SEQ ID NO: 284 can be referred to for portions other than amino acids of the important positions in the PPR motifs. A target protein may also be designed by using a naturally occurring type sequence or existing sequence as a whole, and replacing only amino acids of the important positions.

Examples of naturally occurring type sequences and existing sequences usable for such design as described above are shown below.
A protein consisting any one of the amino acid sequences of SEQ ID NOS: 1 to 5.
A protein consisting any one of the amino acid sequences of SEQ ID NOS: 291 to 308.
A protein having any one amino acid sequence selected from the group consisting of the amino acid sequence of the 230 to 541 positions of SEQ ID NO: 1, the amino acid sequence of the 234 to 621 positions of SEQ ID NO: 2, the amino acid sequence of the 106 to 632 positions of SEQ ID NO: 3, the amino acid sequence of the 106 to 632 positions of SEQ ID NO: 4, and the amino acid sequence of the 256 to 624 positions of SEQ ID NO: 5.
Any one PPR motif selected from the group consisting of 9 PPR motifs of the protein consisting of the amino acid sequence of SEQ ID NO: 1, 11 PPR motifs of the protein consisting of the amino acid sequence of SEQ ID NO: 2, 15 PPR motifs of the protein consisting of the amino acid sequence of SEQ ID NO: 3, 10 PPR motifs of the protein consisting of the amino acid sequence of SEQ ID NO: 4, and 11 PPR motifs of the protein consisting of the amino acid sequence of SEQ ID NO: 5.
A protein having any one amino acid sequence selected from the group consisting of the amino acid sequence of the 167 to 482 positions of SEQ ID NO: 291, the amino acid sequence of the 156 to 575 positions of SEQ ID NO: 292, the amino acid sequence of the 243 to 554 positions of SEQ ID NO: 293, the amino acid sequence of the 140 to 489 positions of SEQ ID NO: 294, the amino acid sequence of the 78 to 419 positions of SEQ ID NO: 295, the amino acid sequence of the 122 to 545 positions of SEQ ID NO: 296, the amino acid sequence of the 256 to 624 positions of SEQ ID NO: 297, the amino acid sequence of the 48 to 362 positions of SEQ ID NO: 298, the amino acid sequence of the 198 to 689 positions of SEQ ID NO: 299, the amino acid sequence of the 89 to 578 positions of SEQ ID NO: 300, the amino acid sequence of the 470 to 911 positions of SEQ ID NO: 301, the amino acid sequence of the 156 to 575 positions of SEQ ID NO: 302, the amino acid sequence of the 108 to 775 positions of SEQ ID NO: 303, the amino acid sequence of the 226 to 1137 positions of SEQ ID NO: 304, the amino acid sequence of the 145 to 496 positions of SEQ ID NO: 305, the amino acid sequence of the 104 to 538 positions of SEQ ID NO: 306, the amino acid sequence of the 151 to 502 positions of SEQ ID NO: 307, and the amino acid sequence of the 274 to 660 positions of SEQ ID NO: 308.
Any one PPR motif selected from the group consisting of 9 PPR motifs of the protein consisting of the amino acid sequence SEQ ID NO: 291, 6 PPR motifs of the protein consisting of the amino acid sequence of SEQ ID NO: 292, 9 PPR motifs of the protein consisting of the amino acid sequence of SEQ ID NO: 293, 10 PPR motifs of the protein consisting of the amino acid sequence of SEQ ID NO: 294, 9 PPR motifs of the protein consisting of the amino acid sequence of SEQ ID NO: 295, 12 PPR motifs of the protein consisting of the amino acid sequence of SEQ ID NO: 296,10 PPR motifs of the protein consisting of the amino acid sequence of SEQ ID NO: 297,9 PPR motifs of the protein consisting of the amino acid sequence of SEQ ID NO: 298, 14 PPR motifs of the protein consisting of the amino acid sequence of SEQ ID NO: 299, 14 PPR motifs of the protein consisting of the amino acid sequence of SEQ ID NO: 300, 10 PPR motifs of the protein consisting of the amino acid sequence of SEQ ID NO: 301, 12 PPR motifs of the protein consisting of the amino acid sequence of SEQ ID NO: 302, 19 PPR motifs of the protein consisting of the amino acid sequence of SEQ ID NO: 303, 25 PPR motifs of the protein consisting of the amino acid sequence of SEQ ID NO: 304, 10 PPR motifs of the protein consisting of the amino acid sequence of SEQ ID NO: 305, 9 PPR motifs of the protein consisting of the amino acid sequence of SEQ ID NO: 306, 10 PPR motifs of the protein consisting of the amino acid sequence of SEQ ID NO: 307, and 11 PPR motifs of the protein consisting of the amino acid sequence of SEQ ID NO: 308.

The present invention provides a novel dPPR protein obtained by the method for designing a dPPR protein, method for imparting a property of binding with a DNA base to a PPR protein, or method of enhancing a property of binding with DNA of a PPR protein, which uses the information explained above. Examples of such a dPPR protein include those containing at least one PPR motif having any one of the amino acid sequences of SEQ ID NOS: 285 to 290. In a preferred embodiment, the protein may contain 2 or more, preferably 2 to 30, more preferably 5 to 25, further preferably 9 to 15, of PPR motifs having any one of the amino acid sequences of SEQ ID NOS: 285 to 290.

The present invention also provides the followings as a novel PPR motif or PPR protein.
A PPR motif having any one of the amino acid sequences of SEQ ID NOS: 7 to 214.
A PPR protein having any one amino acid sequence selected from the group consisting of the amino acid sequence of the 167 to 482 positions of SEQ ID NO: 291, the amino acid sequence of the 156 to 575 positions of SEQ ID NO: 292, the amino acid sequence of the 243 to 554 positions of SEQ ID NO: 293, the amino acid sequence of the 140 to 489 positions of SEQ ID NO: 294, the amino acid sequence of the 78 to 419 positions of SEQ ID NO: 295, the amino acid sequence of the 122 to 545 positions of SEQ ID NO: 296, the amino acid sequence of the 256 to 624 positions of SEQ ID NO: 297, the amino acid sequence of the 48 to 362 positions of SEQ ID NO: 298, the amino acid sequence of the 198 to 689 positions of SEQ ID NO: 299, the amino acid sequence of the 89 to 578 positions of SEQ ID NO: 300, the amino acid sequence of the 470 to 911 positions of SEQ ID NO: 301, the amino acid sequence of the 156 to 575 positions of SEQ ID NO: 302, the amino acid sequence of the 108 to 775 positions of SEQ ID NO: 303, the amino acid sequence of the 226 to 1137 positions of SEQ ID NO: 304, the amino acid sequence of the 145 to 496 positions of SEQ ID NO: 305, the amino acid sequence of the 104 to 538 positions of SEQ ID NO: 306, the amino acid sequence of the 151 to 502 positions of SEQ ID NO: 307, and the amino acid sequence of the 274 to 660 positions of SEQ ID NO: 308.
A protein consisting of any one of the amino acid sequences of SEQ ID NOS: 335 to 361, and a motif contained in it.
A protein consisting of any one of the amino acid sequences of SEQ ID NOS: 424 to 427, and a motif contained in it.

The existing p63 (SEQ ID NO: 1), GUN1 (SEQ ID NO: 2), pTac2 (SEQ ID NO: 3), DG1 (SEQ ID NO: 4), and GRP23 (SEQ ID NO: 5) themselves do not fall within the scope of the present invention. The proteins consisting of the amino acid sequence of SEQ ID NOS: 291 to 308 themselves (At1g10910, At1g26460, At3g15590, At3g59040, At5g10690, At5g24830, At5g67570, At3g42630, At5g42310, At1g12700, At1g30610, At2g35130, At2g41720, At3g18110, At3g53170, At4g21170, At5g48730, and At5g50280) also do not fall within the scope of the present invention.

[Use of dPPR Protein]

The dPPR protein provided by the present invention can be made into a complex by binding a functional region. The functional region generally refers to a part having such a function as a specific biological function exerted in a living body or cell, for example, enzymatic function, catalytic function, inhibitory function, promotion function, etc, or a function as a marker. Such a region consists of, for example, a protein, peptide, nucleic acid, physiologically active substance, or drug.

According to the present invention, by binding a functional region to the PPR protein, the target DNA sequence-binding function exerted by the PPR protein, and the function exerted by the functional region can be exhibited in combination. For example, if a protein having a DNA-cleaving function or a functional domain thereof (for example, nuclease domain of restriction enzyme FokI, SEQ ID NO: 6) is used as the functional region, the complex can function as an artificial DNA-cleaving enzyme.

In order to produce such a complex, methods generally available in this technical field can be used, and there are known a method of synthesizing such a complex as one protein molecule, a method of separately synthesizing two or more members of proteins, and then combining them to form a complex, and so forth.

In the case of the method of synthesizing a complex as one protein molecule, for example, a protein complex can be designed so as to comprise a PPR protein and a cleaving enzyme bound to the C-terminus or N-terminus of the PPR protein via an amino acid linker, an expression vector structure for expressing the protein complex can be constructed, and the target complex can be expressed from the structure. As such a preparation method, the method described in Japanese Patent Unexamined Publication (KOKAI) No. 2013-94148, and so forth can be used.

For binding the PPR protein and the functional region protein, any binding means known in this technical field may be used, including binding via an amino acid linker, binding utilizing specific affinity such as binding between avidin and biotin, binding utilizing another chemical linker, and so forth.

The functional region usable in the present invention refers to a region that can impart any one of various functions such as those for cleavage, transcription, replication, restoration, synthesis, or modification of DNA, and so forth. By choosing the sequence of the PPR motif to define a DNA base sequence as a target, which is the characteristic of the present invention, substantially any DNA sequence may be used as the target, and with such a target, genome editing utilizing the function of the functional region such as those for cleavage, transcription, replication, restoration, synthesis, or modification of DNA can be realized.

For example, when the function of the functional region is a DNA cleavage function, there is provided a complex comprising a PPR protein part prepared according to the present invention and a DNA cleavage region bound together. Such a complex can function as an artificial DNA-cleaving enzyme that recognizes a base sequence of DNA as a target by the PPR protein part, and then cleaves DNA by the DNA cleavage region.

An example of the functional region having a cleavage function usable for the present invention is a deoxyribonuclease (DNase), which functions as an endodeoxyribonuclease. As such a DNase, for example, endodeoxyribonucleases such as DNase A (e.g., bovine pancreatic ribonuclease A, PDB 2AAS), DNase H and DNase I, restriction enzymes derived from various bacteria (for example, FokI) and nuclease domains thereof can be used. Such a complex comprising a PPR protein and a functional region does not exist in the nature, and is novel.

When the function of the functional region is a transcription control function, there is provided a complex comprising a PPR protein part prepared according to the present invention and a DNA transcription control region bound together. Such a complex can function as an artificial transcription control factor, which recognizes a base sequence of DNA as a target by the PPR protein part, and then controls transcription of the target DNA.

The functional region having a transcription control function usable for the present invention may be a domain that activates transcription, or may be a domain that suppresses transcription. Examples of the transcription control domain include VP16, VP64, TA2, STAT-6, and p65. Such a complex comprising a PPR protein and a transcription control domain does not exist in the nature, and is novel.

Further, the complex obtainable according to the present invention may deliver a functional region in a living body or cell in a DNA sequence-specific manner, and allow it to function. It thereby makes it possible to perform modification or disruption in a DNA sequence-specific manner in a living body or cell, like protein complexes utilizing a zinc finger protein (Non-patent documents 1 and 2 mentioned above) or TAL effecter (Non-patent document 3 and Patent document 1 mentioned above), and thus it becomes possible to impart a novel function, i.e., function for cleavage of DNA and genome editing utilizing that function. Specifically, with a PPR protein comprising two or more PPR motifs that can bind with a specific base linked together, a specific DNA sequence can be recognized. Then, genome editing of the recognized DNA region can be realized by the functional region bound to the PPR protein using the function of the functional region.

Furthermore, by binding a drug to the PPR protein that binds to a DNA sequence in a DNA sequence-specific manner, the drug may be delivered to the neighborhood of the DNA sequence as the target. Therefore, the present invention provides a method for DNA sequence-specific delivery of a functional substance.

According to the present invention, the PPR protein shows high DNA-binding ability, and recognizes a specific base on DNA, and as a result, it can be expected to be used to introduce base polymorphism, or treat a disease or condition resulting from a base polymorphism, and in addition, it is considered that the combination of such a PPR protein with such another functional region as mentioned above contribute to modification or improvement of functions for realizing cleavage of DNA for genome editing.

Moreover, an exogenous DNA-cleaving enzyme can be fused to the C-terminus of the PPR protein. Alternatively, by improving binding DNA base selectivity of the PPR motif on the N-terminus side, a DNA sequence-specific DNA-cleaving enzyme can also be constituted. Moreover, such a complex to which a marker part such as GFP is bound can also be used for visualization of a desired DNA in vivo.

### EXAMPLES

### Example 1

### Collection of Novel dPPR Molecules

As known dPPR proteins, there were only P63, GUN1, pTAC2, GRP23, and DG1 described in the prior patent (Patent document 4 mentioned above), and it was difficult to obtain information for generalizing and improving artificial nucleic acid-binding modules based on PPR technique. Therefore, it was then decided to perform screening for PPR proteins having a DNA-binding ability, and thereby increase variety of dPPR proteins. Although the genes of the dPPR molecules accidentally discovered so far contain introns, almost all the rPPR genes do not contain any intron. The total genome sequences ofArabidopsis thaliana as a model plant were analyzed on the basis of the fact mentioned above, and as a result, there were found 42 kinds of PPR genes containing two or more introns. In this example, the DNA-binding abilities of these 42 kinds of potential dPPR molecules were analyzed to attempt identification of novel dPPR molecules.

### Experimental Methods

### 1. Construction of DPPR Expression Vector

From the Institute of Physical and Chemical Research (RIKEN), which holds cDNAs ofArabidopsis thaliana, genes of 10 kinds of the potential dPPRs were obtained. Gene synthesis of GENEWIZ was used for the remaining 32 kinds. The obtained regions corresponding to the PPR motifs of the 42 kinds of the obtained genes were introduced into an expression vector pEU-E01 for wheat cell-free protein synthesis (CellFree Science). Further, a gene encoding thioredoxin and a gene encoding a His-tag were inserted into each gene of potential dPPR molecule on the 5′ end side and the 3′ end side, respectively.

2. Synthesis of dPPR Proteins

mRNAs of the potential dPPR molecules were obtained by using SP6 RNA Polymerase (Promega). The reaction conditions were determined according to the protocol described in the product information. The potential dPPR proteins were obtained by using WEPRO7240H (CellFree Science). The reaction conditions were determined according to the protocol described in the product information.

3. DNA-protein pull-down assay

To each potential dPPR protein, bovine thymus double-stranded DNA cellulose beads (Sigma-Aldrich, 2 mg), and a buffer (20 mM HEPES-KOH, pH 7.9, 60 mM NaCl, 12.5 mM MgCl₂, 0.3% Triton X-100) were added, and the reaction was allowed at 4° C. for 1 hour. The beads were washed 3 times with a washing solution (10 mM Tris-HCl, pH 8.0, 300 mM NaCl, 0.3% Triton X-100), then a 5×SDS-PAGE sample buffer was added to them, and they were heat-treated at 95° C. for 5 minutes to elute the potential dPPR protein.

### 4. Western Blotting

The protein was separated by using 10 to 20% acrylamide gel (ATTO), and transferred to a nitrocellulose membrane. As the transfer buffer, EzFastBlot (ATTO) was used. Blocking was performed with a 0.3% skim milk solution, and the reaction with 0.5 μg/ml of HRP-labeled anti-His-tag antibody (MBL) was allowed at room temperature for 1 hour. For the detection, Immobilon Chemiluminescent HRP Substrate (Millipore) was used. For the detection of the chemiluminescence, VersaDoc (BioRad) was used.

### RESULTS AND DISCUSSION

The DNA-binding powers of the potential dPPR proteins were compared with that of known rPPR OTP80 (Hammani et al., A Study of New Arabidopsis Chloroplast RNA Editing Mutants Reveals General Features of Editing Factors and Their Target Sites, The Plant Cell, Vol. 21:3686-3699, 2009) used as a negative control. The comparison with OTP80 was performed by using t-test performed for numerical values standardized by dividing luminescence intensity of each pulled down protein with that obtained with input 1% at 5% significance level (p<0.06). As a result, significant differences were observed for 18 kinds of the potential dPPRs. These results revealed that these 18 kinds of PPR proteins are dPPR proteins. The sequences of the PPR motifs of the 18 kinds of dPPR proteins are shown in the following tables (mentioned in the order of 1, 2, 3 . . . ).

TABLE 1-1

| | Motif NO. | Position | Sequence | SEQ ID NO.: |
|---|---|---|---|---|
| At1g10910 | 1 | 167-201 | YICNSILSCLVKNOKLDSCIKLEDQMKRDGLKPDV | 7 |
| | 2 | 202-237 | VTYNTLLAGCIKVKNGYPKAIELIGELPHNGIQMDS | 8 |
| | 3 | 238-272 | VMYGTVLAICASNGRSEEAENFIQQMKVEGHSPNI | 9 |
| | 4 | 273-307 | YHYSSLLNSYSWKGDYKKADELMTEMKSIGLVPNK | 10 |
| | 5 | 308-342 | VMMTTLLKVYIKGGLFDRSRELLSELESAGYAENE | 11 |
| | 6 | 343-377 | MPYCMLMDGLSKAGKLEFARSIFDDMKGKGVRSDG | 12 |
| | 7 | 378-412 | YANSIMISALCRSKRFKEAKELSRDSETTYEKCDL | 13 |
| | 8 | 413-447 | VMLNTMLCAYCRAGEMESVMRMMKKMDEQAVSPDY | 14 |
| | 9 | 448-482 | NTFHILIKYFIKEKLHLLAYQTTLDMHSKGHRLEE | 15 |
| At1g26460 | 1 | 156-191 | NLYNHYLRANLMMGASAGDMLDLVAPMEEFSVEPNT | 16 |
| | 2 | 192-228 | ASYNLVLKAMYQARETEAAMKLLERMLLLGKDSLPDD | 17 |
| | 3 | 229-263 | ESYDLVIGMHEGVGKNDEAMKVMDTALKSGYMLST | 18 |
| | 4 | 470-505 | AALNCIILGCANTWDLDRAYQTFEAISASFGLTPNI | 19 |
| | 5 | 506-540 | DSYNALLYAFGKVKKTFEATNVFEHLVSIGVKPDS | 20 |
| | 6 | 541-575 | RTYSLLVDAHLINRDPKSALTVVDDMIKAGFEPSR | 21 |
| At3g15590 | 1 | 243-277 | VVYRTLLANCVLKHHVNKAEDIFNKMKELKFPTSV | 22 |
| | 2 | 278-311 | FACNQLLLLYSMHDRKKISDVLLLMERENIKPSR | 23 |
| | 3 | 312-346 | ATYHFLINSKGLAGDITGMEKIVETIKEEGIELDP | 24 |
| | 4 | 347-381 | ELQSILAKYYIRAGLKERAQDLMKEIEGKGLQQTP | 25 |
| | 5 | 382-413 | WVCRSLLPLYADIGDSDNVRRLSRFVDQNPRY | 26 |
| | 6 | 414-448 | DNCISAIKAWGKLKEVEFAEAVFERLVEKYKIFPM | 27 |
| | 7 | 449-483 | MPYFALMEIYTENKMLAKGRDLVKRMGNAGIAIGP | 28 |
| | 8 | 484-519 | STWHALVKLYIKAGEVGKAELILNRATKDNKMRPMF | 29 |
| | 9 | 520-554 | TTYMAILEEYAKRGDVHNTEKVFMKMKRASYAAQL | 30 |
| At3g59040 | 1 | 140-174 | IDELMLITAYGKLGNENGAERVLSVLSKMGSTPNV | 31 |
| | 2 | 175-209 | ISYTALMESYGRGGKCNNAFAIERRMQSSGPEPSA | 32 |
| | 3 | 210-247 | ITYQIILKTFVEGDKEKEAFEVFETLLDEKKSPLKPDQ | 33 |
| | 4 | 248-282 | KMYHMMIYMYKKAGNYEKARKVESSMVGKGVPQST | 34 |
| | 5 | 283-314 | VTYNSLMSFETSYKEVSKIYDQMQRSDIQPDV | 35 |
| | 6 | 315-349 | VSYALLIKAYGRARREEEALSVFEEMLDAGVRPTH | 36 |
| | 7 | 350-384 | KAYNILLDAFAISGMVEQAKTVEKSMRRDRIFPDL | 37 |
| | 8 | 385-419 | WSYTTMLSAYVNASDMEGAEKFFKRIKVDGFEPNI | 38 |
| | 9 | 420-454 | VTYGTLIKGYAKANDVEKMMEVYEKMRLSGIKANQ | 39 |
| | 10 | 455-489 | TILTTIMDASGRCKNEGSALGWYKEMESCGVPPDQ | 40 |
| At5g10690 | 1 | 78-113 | IVMNSVLEACVHCGNIDLALRMEHEMAEPGGIGVDS | 41 |
| | 2 | 114-152 | ISYATILKGLGKARRIDEAFQMLETIFYGTAAGTPKLSS | 42 |
| | 3 | 153-190 | SLIYGLLDALINAGDLRRANGLLARYDILLLDHGTPSV | 43 |
| | 4 | 191-225 | LIYNLLMKGYVNSESPQAAINLLDEMLRLRLEPDR | 44 |
| | 5 | 226-267 | LTYNTLIHACIKCGDLDAAMKFENDMKEKAFFYYDDFLQPDV | 45 |
| | 6 | 268-303 | VTYTTLVKGFGDATDLLSLQEIFLEMKLCENVFIDR | 46 |
| | 7 | 304-343 | TAFTAVVDAMLKCGSTSGALCVFGEILKRSGANEVLRPKP | 47 |
| | 8 | 344-383 | HLYLSMMRAFAVQGDYGMVRNLYLRLWPDSSGSISKAVQQ | 48 |
| | 9 | 384-419 | EADNLLMEAALNDGQLDEALGILLSIVRRWKTIPWT | 49 |
| At5g24830 | 1 | 122-156 | SIHSSIMRDLCLQGKLDAALWLRKKMIYSGVIPGL | 50 |
| | 2 | 157-191 | ITHNHLLNGLCKAGYIEKADGLVREMREMGPSPNC | 51 |
| | 3 | 192-226 | VSYNTLIKGLCSVNNVDKALYLENTMNKYGIRPNR | 52 |
| | 4 | 227-265 | VTCNIIVHALCQKGVIGNNNKKLLEEILDSSQANAPLDI | 53 |
| | 5 | 266-300 | VICTILMDSCFKNGNVVQALEVWKEMSQKNVPADS | 54 |
| | 6 | 301-335 | VVYNVIIRGLCSSGNMVAAYGFMCDMVKRGVNPDV | 55 |
| | 7 | 336-370 | FTYNTLISALCKEGKFDEACDLHGTMQNGGVAPDQ | 56 |
| | 8 | 371-405 | ISYKVIIQGLCIHGDVNRANEFLLSMLKSSLLPEV | 57 |
| | 9 | 406-440 | LLWNVVIDGYGRYGDTSSALSVLNLMLSYGVKPNV | 58 |
| | 10 | 441-475 | YTNNALIHGYVKGGRLIDAWWVKNEMRSTKIHPDT | 59 |
| | 11 | 476-510 | TTYNLLLGAACTLGHLRLAFQLYDEMLRRGCQPDI | 60 |
| | 12 | 511-545 | ITYTELVRGLCWKGRLKKAESLLSRIQATGITIDH | 61 |

TABLE 1-2

| | Motif | | | SEQ ID |
|---|---|---|---|---|
| | NO. | Position | Sequence | NO.: |
| At5g67570 | 1 | 256-291 | FVYTKLLSVLGFARRPQEALQIENQMLGDRQLYPDM | 62 |
| | 2 | 292-341 | AAYHCIAVTLGQAGLLKELLKVIERMRQKPTKLTKNLRQKNWDPVLEPDL | 63 |
| | 3 | 342-376 | VVYNAILNACVPTLQWKAVSWVFVELRKNGLRPNG | 64 |
| | 4 | 377-411 | ATYGLAMEVMLESGKFDRVHDFFRKMKSSGEAPKA | 65 |
| | 5 | 412-446 | ITYKVLVRALWREGKIEFAVEAVRDMEQKGVIGTG | 66 |
| | 6 | 447-482 | SVYYELACCLCNNGRWCDAMLEVGRMKRLENCRPLE | 67 |
| | 7 | 483-516 | ITFTGLIAASLNGGHVDDCMAIFQYMKDKCDPNI | 68 |
| | 8 | 517-554 | GTANMMLKVYGRNDMFSEAKELFEEIVSRKETHLVPNE | 69 |
| | 9 | 555-589 | YTYSFMLEASARSLQWEYFEHVYQTMVLSGYQMDQ | 70 |
| | 10 | 590-624 | TKHASMLIEASRAGKWSLLEHAFDAVLEDGEIPHP | 71 |
| At3g42630 | 1 | 48-82 | VDYAPLVQTLSQRRLPDVAHEIFLQTKSVNLLPNY | 72 |
| | 2 | 83-117 | RTLCALMLCFAENGFVLRARTIWDEIINSCFVPDV | 73 |
| | 3 | 118-152 | FVVSKLISAYEQFGCFDEVAKITKDVAARHSKLLP | 74 |
| | 4 | 153-187 | VVSSLAISCFGKNGQLELMEGVIEEMDSKGVLLEA | 75 |
| | 5 | 188-222 | ETANVIVRYYSFEGSLDKMEKAYGRVKKEGIVIEE | 76 |
| | 6 | 223-257 | EFIRAVVLAYLKQRKFYRLREFLSDVGLGRRNLGN | 77 |
| | 7 | 258-292 | MLWNSVLLSYAADFKMKSLQREFIGMLDAGFSPDL | 78 |
| | 8 | 293-327 | TTFNIRALAFSRMALFWDLHLTLEHMRRLNIVPDL | 79 |
| | 9 | 328-362 | VTFGCVVDAYMDKRLARNLEFVYNRMNLDDSPLVL | 80 |
| At5g42310 | 1 | 198-232 | LTYNALIGACARNNDIEKALNLIAKMRQDGYQSDF | 81 |
| | 2 | 233-269 | VNYSLVIQSLTRSNKIDSVMLLRLYKEIERDKLELDV | 82 |
| | 3 | 270-304 | QLVNDIIMGFAKSGDPSKALQLLGMAQATGLSAKT | 83 |
| | 4 | 305-339 | ATLVSIISALADSGRTLEAEALFEELRQSGIKPRT | 84 |
| | 5 | 340-374 | RAYNALLKGYVKTGPLKDAESMVSEMEKRGVSPDE | 85 |
| | 6 | 375-409 | HTYSLLIDAYVNAGRWESARIVLKEMEAGDVQPNS | 86 |
| | 7 | 410-444 | FVFSRLLAGFRDRGEWQKTFQVLKEMKSIGVKPDR | 87 |
| | 8 | 445-479 | QFYNVVIDTEGKENCLDHAMTTFDRMLSEGIEPDR | 88 |
| | 9 | 480-514 | VTWNTLIDCHCKHGRHIVAEEMFEAMERRGCLPCA | 89 |
| | 10 | 515-549 | TTYNIMINSYGDQERWDDMKRLLGKMKSQGILPNV | 90 |
| | 11 | 550-584 | VTHTTLVDVYGKSGRENDAIECLEEMKSVGLKPSS | 91 |
| | 12 | 585-619 | TMYNALINAYAQRGLSEQAVNAFRVMTSDGLKPSL | 92 |
| | 13 | 620-654 | LALNSLINAFGEDRRDAEAFAVLQYMKENGVKPDV | 93 |
| | 14 | 655-689 | VTYTTLMKALIRVDKFQKVPVVYEEMIMSGCKPDR | 94 |
| At1g12700 | 1 | 89-123 | VDFSRFFSAIARTKQFNLVLDFCKQLELNGIAHNI | 95 |
| | 2 | 124-158 | YTLNIMINCFCRCCKTCFAYSVLGKVMKLGYEPDT | 96 |
| | 3 | 159-193 | TTENTLIKGLFLEGKVSEAVVLVDRMVENGCQPDV | 97 |
| | 4 | 194-228 | VTYNSIVNGICRSGDTSLALDLLRKMEERNVKADV | 98 |
| | 5 | 229-263 | FTYSTIIDSLCRDGCIDAAISLEKEMETKGIKSSV | 99 |
| | 6 | 264-298 | VTYNSLVRGLCKAGKWNDGALLLKDMVSREIVPNV | 100 |
| | 7 | 299-333 | ITENVLLDVFVKEGKLQEANELYKEMITRGISPNI | 101 |
| | 8 | 334-368 | ITYNTLMDGYCMQNRLSEANNMLDLMVRNKCSPDI | 102 |
| | 9 | 369-403 | VTFTSLIKGYCMVKRVDDGMKVERNISKRGLVANA | 103 |
| | 10 | 404-438 | VTYSILVQGFCQSGKIKLAEELFQEMVSHGVLPDV | 104 |
| | 11 | 439-473 | MTYGILLDGLCDNGKLEKALEIFEDLQKSKMDLGI | 105 |
| | 12 | 474-508 | VMYTTIIEGMCKGGKVEDAWNLFCSLPCKGVKPNV | 106 |
| | 13 | 509-543 | MTYTVMISGLCKKGSLSEANILLRKMEEDGNAPND | 107 |
| | 14 | 544-578 | CTYNTLIRAHLRDGDLTASAKLIEEMKSCGESADA | 108 |
| At1g30610 | 1 | 470-507 | YTVMRLIHFLGKLGNWRRVLQVIEWLQRQDRYKSNKIR | 109 |
| | 2 | 508-538 | IIYTTALNVLGKSRRPVEALNVEHAMLLQISSYPDM | 110 |
| | 3 | 544-593 | VAYRSIAVTLGQAGHIKELFYVIDTMRSPPKKKEKPTTLEKWDPRLEPDV | 111 |
| | 4 | 594-628 | VVYNAVLNACVQRKQWEGAFWVLQQLKQRGQKPSP | 112 |
| | 5 | 629-662 | VTYGLIMEVMLACEKYNLVHEFFRKMQKSSIPNA | 113 |
| | 6 | 663-697 | LAYRVLVNTLWKEGKSDEAVHTVEDMESRGIVGSA | 114 |
| | 7 | 761-794 | VTYTGLTQACVDSGNIKNAAYIEDQMKKVCSPNL | 115 |
| | 8 | 795-841 | VTCNIMLKAYLQGGLFEEARELFQKMSEDGNHIKNSSDFESRVLPDT | 116 |
| | 9 | 842-876 | YTENTMLDTCAEQEKWDDEGYAYREMLRHGYHENA | 117 |
| | 10 | 877-911 | KRHLRMVLEASRAGKEEVMEATWEHMRRSNRIPPS | 118 |

TABLE 1-3

| | Motif | | | SEQ |
|---|---|---|---|---|
| | NO. | Position | Sequence | ID NO.: |
| At2g35130 | 1 | 156-190 | ICFNLLIDAYGQKFQYKEAESLYVQLLESRYVPTE | 119 |
| | 2 | 191-225 | DTYALLIKAYCMAGLIERAEVVLVEMQNHHVSPKT | 120 |
| | 3 | 229-264 | TVYNAYIEGLMKRKGNTEFAIDVFQRMKRDRCKPTT | 121 |
| | 4 | 265-299 | ETYNLMINLYGKASKSYMSWKLYCEMRSHQCKPNI | 122 |
| | 5 | 300-334 | CTYTALVNAFAREGLCEKAFFIFEQLQEDGLEPDV | 123 |
| | 6 | 335-369 | YVYNALMESYSRAGYPYGAAEIFSLMQHMGCEPDR | 124 |
| | 7 | 370-404 | ASYNIMVDAYGRAGLHSDAEAVFEEMKRLGIAPTM | 125 |
| | 8 | 405-439 | KSHMLLLSAYSKARDVTKCEAIVKEMSENGVEPDT | 126 |
| | 9 | 440-474 | FVLNSMLNLYGRLGQFTKMEKILAEMENGPCTADI | 127 |
| | 10 | 475-509 | STYNILINIYGKAGFLERIEELFVELKEKNFRPDV | 128 |
| | 11 | 510-544 | VTWTSRIGAYSRKKLYVKCLEVFEEMIDSGCAPDG | 129 |
| | 12 | 545-575 | GTAKVLLSACSSEEQVEQVTSVLRTMHKGVT | 130 |
| At2g41720 | 1 | 108-143 | KNFPVLIRELSRRGCIELCVNVEKWMKIQKNYCARN | 131 |
| | 2 | 144-178 | DIYNMMIRLHARHNWVDQARGLFFEMQKWSCKPDA | 132 |
| | 3 | 179-213 | ETYDALINAHGRAGQWRWAMNLMDDMLRAAIAPSR | 133 |
| | 4 | 214-248 | STYNNLINACGSSGNWREALEVCKKMTDNGVGPDL | 134 |
| | 5 | 249-283 | VTHNIVLSAYKSGRQYSKALSYFELMKGAKVRPDT | 135 |
| | 6 | 284-320 | TTENIIIYCLSKLGQSSQALDLENSMREKRAECRPDV | 136 |
| | 7 | 321-355 | VTFTSIMHLYSVKGEIENCRAVFEAMVAEGLKPNI | 137 |
| | 8 | 356-390 | VSYNALMGAYAVHGMSGTALSVLGDIKQNGIIPDV | 138 |
| | 9 | 391-425 | VSYTCLLNSYGRSRQPGKAKEVFLMMRKERRKPNV | 139 |
| | 10 | 426-460 | VTYNALIDAYGSNGFLAEAVEIFRQMEQDGIKPNV | 140 |
| | 11 | 461-495 | VSVCTLLAACSRSKKKVNVDTVLSAAQSRGINLNT | 141 |
| | 12 | 496-530 | AAYNSAIGSYINAAELEKAIALYQSMRKKKVKADS | 142 |
| | 13 | 531-565 | VTFTILISGSCRMSKYPEAISYLKEMEDLSIPLTK | 143 |
| | 14 | 566-600 | EVYSSVLCAYSKQGQVTEAESIFNQMKMAGCEPDV | 144 |
| | 15 | 601-635 | IAYTSMLHAYNASEKWGKACELFLEMEANGIEPDS | 145 |
| | 16 | 636-670 | IACSALMRAFNKGGQPSNVFVLMDLMREKEIPFTG | 146 |
| | 17 | 671-705 | AVFFEIFSACNTLQEWKRAIDLIQMMDPYLPSLSI | 147 |
| | 18 | 706-740 | GLTNQMLHLFGKSGKVEAMMKLFYKIIASGVGINL | 148 |
| | 19 | 741-775 | KTYAILLEHLLAVGNWRKYIEVLEWMSGAGIQPSN | 149 |
| At3g18110 | 1 | 226-260 | QVYNAMMGVYSRSGKESKAQELVDAMRQRGCVPDL | 150 |
| | 2 | 261-297 | ISENTLINARLKSGGLTPNLAVELLDMVRNSGLRPDA | 151 |
| | 3 | 298-332 | ITYNTLLSACSRDSNLDGAVKVFEDMEAHRCQPDL | 152 |
| | 4 | 333-367 | WTYNAMISVYGRCGLAAFAERLFMELELKGFFPDA | 153 |
| | 5 | 368-402 | VTYNSLLYAFARERNTEKVKEVYQQMQKMGFGKDE | 154 |
| | 6 | 403-438 | MTYNTIIHMYGKQGQLDLALQLYKDMKGLSGRNPDA | 155 |
| | 7 | 439-473 | ITYTVLIDSLGKANRTVEAAALMSEMLDVGIKPTL | 156 |
| | 8 | 474-508 | QTYSALICGYAKAGKREFAEDTESCMLRSGTKPDN | 157 |
| | 9 | 509-543 | LAYSVMLDVLLRGNETRKAWGLYRDMISDGHTPSY | 158 |
| | 10 | 544-574 | TLYELMILGLMKENRSDDIQKTIRDMEELCG | 159 |
| | 11 | 610-644 | DTLLSILGSYSSSGRHSEAFELLEFLKEHASGSKR | 160 |
| | 12 | 645-681 | LITEALIVLHCKVNNLSAALDEYFADPCVHGWCFGSS | 161 |
| | 13 | 682-716 | TMYETLLHCCVANEHYAEASQVFSDLRLSGCEASE | 162 |
| | 14 | 717-752 | SVCKSMVVVYCKLGFPETAHQVVNQAETKGFHFACS | 163 |
| | 15 | 753-787 | PMYTDIIEAYGKQKLWQKAESVVGNLRQSGRTPDL | 164 |
| | 16 | 788-822 | KTWNSLMSAYAQCGCYFRARAIENTMMRDGPSPTV | 165 |
| | 17 | 823-857 | ESINILLHALCVDGRLEELYVVVEELQDMGFKISK | 166 |
| | 18 | 858-892 | SSILLMLDAFARAGNIFEVKKIYSSMKAAGYLPTI | 167 |
| | 19 | 893-927 | RLYRMMIELLCKGKRVRDAEIMVSEMEEANFKVEL | 168 |
| | 20 | 928-962 | AIWNSMLKMYTAIEDYKKTVQVYQRIKETGLEPDE | 169 |
| | 21 | 963-997 | TTYNTLIIMYCRDRRPEEGYLLMQQMRNLGLDPKL | 170 |
| | 22 | 998-1032 | DTYKSLISAFGKQKCLEQAEQLFEELLSKGLKLDR | 171 |
| | 23 | 1033-1067 | SFYHTMMKISRDSGSDSKAEKLLQMMKNAGIEPTL | 172 |
| | 24 | 1068-1102 | ATMHLLMVSYSSSGNPQEAEKVLSNLKDTEVELTT | 173 |
| | 25 | 1103-1137 | LPYSSVIDAYLRSKDYNSGIERLLEMKKEGLEPDH | 174 |

TABLE 1-4

| | Motif | | | SEQ |
|---|---|---|---|---|
| | NO. | Position | Sequence | ID NO.: |
| At3g53170 | 1 | 145-179 | KTYTKLFKVLGNCKQPDQASLLFEVMLSEGLKPTI | 175 |
| | 2 | 180-215 | DVYTSLISVYGKSELLDKAFSTLEYMKSVSDCKPDV | 176 |
| | 3 | 216-250 | FTFTVLISCCCKLGRFDLVKSIVLEMSYLGVGCST | 177 |
| | 4 | 251-286 | VTYNTIIDGYGKAGMFEEMESVLADMIEDGDSLPDV | 178 |
| | 5 | 287-321 | CTLNSIIGSYGNGRNMRKMESWYSREQLMGVQPDI | 179 |
| | 6 | 322-356 | TTFNILILSFGKAGMYKKMCSVMDFMEKRFFSLTT | 180 |
| | 7 | 357-391 | VTYNIVIETFGKAGRIEKMDDVFRKMKYQGVKPNS | 181 |
| | 8 | 392-426 | ITYCSLVNAYSKAGLVVKIDSVLRQIVNSDVVLDT | 182 |
| | 9 | 427-461 | PFFNCIINAYGQAGDLATMKELYIQMEERKCKPDK | 183 |
| | 10 | 462-496 | ITFATMIKTYTAHGIFDAVQELEKQMISSDIGKKRL | 184 |
| At4g21170 | 1 | 104-153 | KSHCRVIEVAAESGLLERAEMLLRPLVETNSVSLVVGEMHRWFEGEVSLS | 185 |
| | 2 | 154-188 | VSLSLVLEYYALKGSHHNGLEVEGFMRRLRLSPSQ | 186 |
| | 3 | 189-223 | SAYNSLLGSLVKENQFRVALCLYSAMVRNGIVSDE | 187 |
| | 4 | 254-288 | KIYTNLVECYSRNGEFDAVESLIHEMDDKKLELSF | 188 |
| | 5 | 289-323 | CSYGCVLDDACRLGDAEFIDKVLCLMVEKKFVTLG | 189 |
| | 6 | 362-397 | STYGCMLKALSRKKRTKEAVDVYRMICRKGITVLDE | 190 |
| | 7 | 398-433 | SCYIEFANALCRDDNSSEEEEELLVDVIKRGKEDGN | 191 |
| | 8 | 470-505 | NAYNAVLDRLMMRQKEMVEEAVVVFEYMKEINSVNS | 192 |
| | 9 | 506-538 | KSFTIMIQGLCRVKEMKKAMRSHDEMLRLGLKP | 193 |
| At5g48730 | 1 | 151-185 | GIYVKLIVMLGKCKQPEKAHELFQEMINEGCVVNH | 194 |
| | 2 | 186-221 | EVYTALVSAYSRSGRFDAAFTLLERMKSSHNCQPDV | 195 |
| | 3 | 222-256 | HTYSILIKSFLQVFAFDKVQDLLSDMRRQGIRPNT | 196 |
| | 4 | 257-292 | ITYNTLIDAYGKAKMFVEMESTLIQMLGEDDCKPDS | 197 |
| | 5 | 293-327 | WTMNSTLRAFGGNGQIEMMENCYEKFQSSGIEPNI | 198 |
| | 6 | 328-362 | RTFNILLDSYGKSGNYKKMSAVMEYMQKYHYSWTI | 199 |
| | 7 | 363-397 | VTYNVVIDAFGRAGDLKQMEYLFRLMQSERIFPSC | 200 |
| | 8 | 398-432 | VTLCSLVRAYGRASKADKIGGVLRFIENSDIRLDL | 201 |
| | 9 | 433-467 | VFFNCLVDAYGRMEKFAEMKGVLELMEKKGEKPDK | 202 |
| | 10 | 468-502 | ITYRTMVKAYRISGMTTHVKELHGVVESVGEAQVV | 203 |
| At5g50280 | 1 | 274-308 | RLYNAAISGLSASQRYDDAWEVYEAMDKINVYPDN | 204 |
| | 2 | 309-344 | VTCAILITTLRKAGRSAKEVWEIFEKMSEKGVKWSQ | 205 |
| | 3 | 345-379 | DVFGGLVKSFCDEGLKEEALVIQTEMEKKGIRSNT | 206 |
| | 4 | 380-414 | IVYNTLMDAYNKSNHIEEVEGLFTEMRDKGLKPSA | 207 |
| | 5 | 415-449 | ATYNILMDAYARRMQPDIVETLLREMEDLGLEPNV | 208 |
| | 6 | 450-485 | KSYTCLISAYGRTKKMSDMAADAFLRMKKVGLKPSS | 209 |
| | 7 | 486-520 | HSYTALIHAYSVSGWHEKAYASFEEMCKEGIKPSV | 210 |
| | 8 | 521-555 | ETYTSVLDAFRRSGDTGKLMEIWKLMLREKIKGTR | 211 |
| | 9 | 556-590 | ITYNTLLDGFAKQGLYIEARDVVSEFSKMGLQPSV | 212 |
| | 10 | 591-625 | MTYNMLMNAYARGGQDAKLPQLLKEMAALNLKPDS | 213 |
| | 11 | 626-660 | ITYSTMIYAFVRVRDFKRAFFYHKMMVKSGQVPDP | 214 |

### Example 2

### Analysis of dPPR Motif-Specific Amino Acid Sequences

On the basis of the amino acid sequence information of the modules of the dPPR proteins identified in Example 1, dPPR motif-specific amino acid sequences were analyzed.

First, 9 kinds of the dPPR proteins were selected from the 18 kinds of dPPR proteins identified in Example 1 in order to approximately match the number of them with the number of motifs of rPPR proteins used in the F test. Specifically, on the basis of the numerical values obtained from the comparison of the DNA-binding power with that of OTP80 performed by the t-test, the dPPR proteins were classified into 3 groups of those showing the values of 0.05 to 0.01, 0.01 to 0.001, and <0.001, and 3 kinds of proteins were randomly selected from each group to select 9 kinds of the proteins. The occurrence frequencies of amino acids in PPR motifs of the 9 kinds of dPPR molecules and the known 5 rPPR molecules mentioned in the following tables (mentioned in the order of 1, 2, 3 . . . ) were compared at every position to attempt identification of positions of amino acids characterizing the dPPR proteins. For the comparison, the F test was used at a significance level of 5% (p<0.06).

TABLE 2-1

| | Motif | | SEQ |
|---|---|---|---|
| | NO. | Sequence | ID NO.: |
| At3g61360 | 1 | DSFEKTLHILARMRYFDQAWALMAEVRKDYPNLLSF | 215 |
| | 2 | KSMSILLCKIAKEGSYEETLEAFVKMEKEIFRKKEGV | 216 |
| | 3 | DEFNILLRAFCTEREMKEARSIFEKLHSRFNPDV | 217 |
| | 4 | KTMNILLLGFKEAGDVTATELFYHEMVKRGFKPNS | 218 |
| | 5 | VTYGIRIDGFCKKRNFGEALRLFEDMDRLDFDITV | 219 |
| | 6 | QILTTLIHGSGVARNKIKARQLFDEISKRGLTPDC | 220 |
| | 7 | GAYNALMSSLMKCGDVSGAIKVMKEMEEKGIEPDS | 221 |
| | 8 | VTFHSMFIGMMKSKEFGENGVCEYYQKMKERSLVPKT | 222 |
| | 9 | PTIVMLMKLECHVGEVNLGLDLWKYMLEKGYCPHG | 223 |
| AT5G11310 | 1 | SLEDSVVNSLCKAREFFIAWSLVFDRVRSDEGSNLVSA | 224 |
| | 2 | DTFIVLIRRYARAGMVQQAIRAFEFARSYEPVCKSATEL | 225 |
| | 3 | RLLEVLLDALCKEGHVREASMYLERIGGTMDSNWVPSV | 226 |
| | 4 | RIFNILLNGWERSRKLKQAEKLWEEMKAMNVKPTV | 227 |
| | 5 | VTYGTLIEGYCRMRRVQIAMEVLEEMKMAEMEINF | 228 |
| | 6 | MVFNPIIDGLGEAGRLSEALGMMERFFVCESGPTI | 229 |
| | 7 | VTYNSLVKNECKAGDLPGASKILKMMMTRGVDPTT | 230 |
| | 8 | TTYNHFFKYFSKHNKTEEGMNLYFKLIEAGHSPDR | 231 |
| | 9 | LTYHLILKMLCEDGKLSLAMQVNKEMKNRGIDPDL | 232 |
| | 10 | LTTTMLIHLLCRLEMLEEAFEEFDNAVRRGIIPQY | 233 |
| | 11 | ITFKMIDNGLRSKGMSDMAKRLSSLMSSLPHSKKL | 234 |
| AT1G06710 | 1 | PVYNALVDLIVRDDDEKVPEEFLQQIRDDDKEVFG | 235 |
| | 2 | EFLNVLVRKHCRNGSFSIALEELGRLKDFRFRPSR | 236 |
| | 3 | STYNCLIQAFLKADRLDSASLIHREMSLANLRMDG | 237 |
| | 4 | FTLRCFAYSLCKVGKWREALTLVETENFVPDT | 238 |
| | 5 | VEYTKLISGLCEASLFEEAMDFLNRMRATSCLPNV | 239 |
| | 6 | VTYSTLLCGCLNKKQLGRCKRVLNMMMMEGCYPSP | 240 |
| | 7 | KIENSLVHAYCTSGDHSYAYKLLKKMVKCGHMPGY | 241 |
| | 8 | VVYNILIGSICGDKDSLNCDLLDLAEKAYSEMLAAGVVLNK | 242 |
| | 9 | INVSSFTRCLCSAGKYEKAFSVIREMIGQGFIPDT | 243 |
| | 10 | STYSKVLNYLCNASKMELAELLFEEMKRGGLVADV | 244 |
| | 11 | YTYTIMVDSECKAGLIEQARKWENEMREVGCTPNV | 245 |
| | 12 | VTYTALIHAYLKAKKVSYANELFETMLSEGCLPNI | 246 |
| | 13 | VTYSALIDGHCKAGQVEKACQIFERMCGSKDVPDVDMYFKQYDDNSERPNV | 247 |
| | 14 | VTYGALLDGFCKSHRVEEARKLLDAMSMEGCEPNQ | 248 |
| | 15 | IVYDALIDGLCKVGKLDEAQEVKTEMSEHGFPATL | 249 |
| | 16 | YTYSSLIDRYFKVKRQDLASKVLSKMLENSCAPNV | 250 |
| | 17 | VIYTEMIDGLCKVGKTDEAYKLMQMMEEKGCQPNV | 251 |
| | 18 | VTYTAMIDGEGMIGKIETCLELLERMGSKGVAPNY | 252 |
| | 19 | VTYRVLIDHCCKNGALDVAHNLLEEMKQTHWPTHT | 253 |
| | 20 | SVYRLLIDNLIKAQRLEMALRLLEEVATFSATLVDYS | 254 |
| | 21 | STYNSLIESLCLANKVETAFQLFSEMTKKGVIPEM | 255 |
| | 22 | QSFCSLIKGLFRNSKISEALLLLDFISHMEIQWIE | 256 |

TABLE 2-2

| | Motif | | SEQ |
|---|---|---|---|
| | NO. | Sequence | ID NO.: |
| At2g18940 | 1 | RAYTTILHAYSRTGKYEKAIDLFERMKEMGPSPTL | 257 |
| | 2 | VTYNVILDVEGKMGRSWRKILGVLDEMRSKGLKEDE | 258 |
| | 3 | FTCSTVLSACAREGLLREAKEFFAELKSCGYEPGT | 259 |
| | 4 | VTYNALLQVFGKAGVYTEALSVLKEMEENSCPADS | 260 |
| | 5 | VTYNELVAAYVRAGFSKEAAGVIEMMTKKGVMPNA | 261 |
| | 6 | ITYTTVIDAYGKAGKEDEALKLEYSMKEAGCVPNT | 262 |
| | 7 | CTYNAVLSLLGKKSRSNEMIKMLCDMKSNGCSPNR | 263 |
| | 8 | ATWNTMLALCGNKGMDKEVNRVEREMKSCGFEPDR | 264 |
| | 9 | DTENTLISAYGRCGSEVDASKMYGEMTRAGENACV | 265 |
| | 10 | TTYNALLNALARKGDWRSGENVISDMKSKGFKPTE | 266 |
| | 11 | TSYSLMLQCYAKGGNYLGIERIENRIKEGQIEPSW | 267 |
| | 12 | MLLRTLLLANFKCRALAGSERAFTLFKKHGYKPDM | 268 |
| | 13 | VIENSMLSIFTRNNMYDQAEGILESIREDGLSPDL | 269 |
| | 14 | VTYNSLMDMYVRRGECWKAFFILKTLEKSQLKPDL | 270 |
| | 15 | VSYNTVIKGFCRRGLMQEAVRMLSEMTERGIRPCI | 271 |
| | 16 | FTYNTEVSGYTAMGMFAFIEDVIECMAKNDCRPNE | 272 |
| | 17 | LTFKMVVDGYCRAGKYSEAMDFVSKIKTFDP | 273 |
| At3g09650 | 1 | AAFNAVLNACANLGDTDKYWKLFEEMSEWDCEPDV | 274 |
| | 2 | LTYNVMIKLCARVGRKELIVEVLERIIDKGIKVCM | 275 |
| | 3 | TTMHSLVAAYVGFGDLRTAERIVQAMREKRRDLCK | 276 |
| | 4 | RIYTTLMKGYMKNGRVADTARMLEAMRRQDDRNSHPDE | 277 |
| | 5 | VTYTTVVSAFVNAGLMDRARQVLAEMARMGVPANR | 278 |
| | 6 | ITYNVLLKGYCKQLQIDRAEDLLREMTEDAGIEPDV | 279 |
| | 7 | VSYNIIIDGGCILIDDSAGALAFFNEMRTRGIAPTK | 280 |
| | 8 | TKISYTTLMKAFAMSGQPKLANRVEDEMMNDPRVKVIDL | 281 |
| | 9 | IAWNMLVEGYCRLGLIEDAQRVVSRMKENGFYPNV | 282 |
| | 10 | ATYGSLANGVSQARKPGDALLLWKEIKERCA | 283 |

From the results of the F test (FIG. 1), there were observed differences in occurrence frequencies for the amino acids of the residues of No. 7 amino acid (A.A.), No. 9 A.A., No. 10 A.A., No. 18 A.A., No. 20 A.A., No. 29 A.A., No. 31 A.A., No. 32 A.A., and No. ii A.A. No. ii A.A. was excluded, since it is a part involved in recognition of a DNA base (Patent document 4 mentioned above).

Then, the occurrence frequencies of the amino acids at these positions were calculated, and amino acids that showed the largest positive differences between dPPR and rPPR were confirmed. As a result, it was found that occurrence frequencies of I as No. 7 A.A., A as No. 9 A.A., Y as No. 10 A.A., K as No. 18 A.A., E as No. 20 A.A., E as No. 29 A.A., I as No. 31 A.A., and K as No. 32 A.A. increased in the dPPR molecules. On the basis of these results, the aforementioned amino acids were determined as dPPR motif-specific amino acid sequences.

The contents (%) of the dPPR specific amino acids in the novel dPPR proteins (9 kinds of the proteins used for the data set) and known rPPRs are shown in the following table.

TABLE 3

| Novel dPPR proteins, known rPPR | | | | | | | | |
|---|---|---|---|---|---|---|---|---|
| | Average | Average | | Known dPPR | | | | |
| | (dPPR) | (rPPR) | Median | P63 | GUN1 | pTAC2 | DG1 | GRP23 |
| AA7I | 0.45 | 0.35 | 0.40 | 0.33 | 0.64 | 0.47 | 0.10 | 0.36 |
| AA9A | 0.49 | 0.23 | 0.36 | 0.11 | 0.45 | 0.47 | 0.40 | 0.27 |
| AA10Y | 0.50 | 0.25 | 0.37 | 0.56 | 0.36 | 0.33 | 0.10 | 0.18 |
| AA18K | 0.29 | 0.09 | 0.19 | 0.44 | 0.09 | 0.13 | 0.00 | 0.09 |
| AA20E | 0.25 | 0.16 | 0.21 | 0.56 | 0.00 | 0.13 | 0.20 | 0.09 |
| AA29E | 0.12 | 0.06 | 0.09 | 0.22 | 0.18 | 0.13 | 0.00 | 0.00 |
| AA31I | 0.23 | 0.10 | 0.16 | 0.00 | 0.45 | 0.40 | 0.00 | 0.00 |
| AA32K | 0.22 | 0.09 | 0.15 | 0.00 | 0.09 | 0.00 | 0.10 | 0.09 |

### Example 3-1

### Establishment of Method for Constructing Artificial Nucleic Acid-Binding Module Based on dPPR Motif-Specific Amino Acid Sequences 1

In this example, the DNA-binding abilities of modified type rPPRs introduced with the dPPR specific amino acid sequences were investigated in order to verify whether the DNA-binding abilities of PPR proteins are increased by the dPPR-specific amino acid sequences. As the base rPPR, the consensus PPR (cPPR) reported in Non-patent document 15 (Coquille et al., 2014, An artificial PPR scaffold for programmable RNA recognition) was used. cPPR is known as an RNA-binding protein (therefore, it may be referred to as crPPR), and it had not been known whether it binds with DNA. For the modification of crPPR, gene synthesis by Genewiz was used. The DNA-binding abilities of the modified type crPPRs were analyzed by the method used in Example 1. The target sequence of crPPR is AAAAAAAA.

Since there was a tendency that AA9A and AA10Y changed within the same motif, they were inserted in combination in this experiment. Since there was also a tendency that AA20E was introduced into a motif preceding that of AA18K, they were inserted in combination. When the contents were calculated from the data obtained from all the dPPRs (18 kinds also including the dPPR protein molecules other than those used for the data set), the content of AA10Y in a motif also having AA9A was 43.75%, and the content of AA18K in a motif next to a motif having AA 20E was 41.3%. The sequences of cPPRs and the modified type PPR motifs prepared in this example are shown in the following table (mentioned in the order of 1, 2, 3 . . . ).

TABLE 4

| | | |
|---|---|---|
| crPPR | VTYTTLISGLGKAGRLEEALELFEEMKEKGIVPNV | SEQ ID NO.: 284 |
| Modified crPPR-1 | VTYTTLISAYGKAGRLEEALELFEEMKEKGIVPNV | SEQ ID NO.: 285 |
| Modified crPPR-2 | VTYTTLISGLGKAGRLEKAEELFEEMKEKGIVPNV | SEQ ID NO.: 286 |
| Modified crPPR-3 | VTYTTLISGLGKAGRLEEALELFEEMKEKGIKPNV | SEQ ID NO.: 287 |
| Modified crPPR-4 | VTYTTLISAYGKAGRLEKAEELFEEMKEKGIVPNV | SEQ ID NO.: 288 |
| Modified crPPR-5 | VTYTTLISAYGKAGRLEEALELFEEMKEKGIKPNV | SEQ ID NO.: 289 |
| Modified crPPR-6 | VTYTTLISAYGKAGRLEKAEELFEEMKEKGIKPNV | SEQ ID NO.: 290 |

### RESULTS AND DISCUSSION

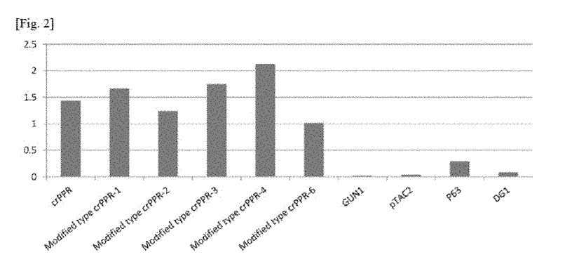

Comparison of the DNA-binding power was performed with values obtained by standardization by dividing luminescence intensity of each pulled-down protein with that obtained with input 3%. The results are shown in FIG. 2.

There were obtained results that the DNA-binding powers of crPPR and all the modified type crPPRs in which each dPPR motif-specific amino acid sequence was inserted were higher than those of GUN1, pTAC2, p63, and DG1, which are naturally occurring dPPR molecules. These results indicate that the dPPR motif-specific amino acid sequences found in this research and development relate to the DNA-binding ability of PPR protein.

On the basis of the above test results obtained in this example, it was discovered that a DNA-binding ability can be imparted to a PPR protein by inserting a dPPR motif-specific amino acid sequence.

### Example 3-2

### Establishment of Method for Constructing Artificial Nucleic Acid-Binding Module Based on dPPR Motif-Specific Amino Acid Sequences 2

The aforementioned cPPR (Non-patent document 15) has an RNA-binding property, but it has A.A. 71 and A.A. 31I. Therefore, there was used a modified version thereof in which these amino acids are replaced with leucine (L) and phenylalanine (F), respectively, with reference to the occurrence frequencies of amino acids in rPPR. In this specification, this modified version is referred to as consensus RNA-binding PPR (7L/31F) (crPPR (7L/31F)). Since there was a tendency that AA9A and AA10Y changed within the same motif, one having them in combination was also examined (the ratio of AA10Y in a motif also having AA9A was 43.75%, when it was calculated from the data obtained from the 18 kinds of dPPRs including the dPPRs other than those used for the data set).

### Experimental Method

1. Construction of Modified Type crPPR Expression Vector

For the genes of crPPR (7L/31F) and the modified versions of the same introduced with a modified type rPPR, the gene synthesis by GENEWIZ was used. Each of the obtained genes was introduced into the expression vector pEU-E01 for wheat cell-free protein synthesis (CellFree Science). A gene encoding thioredoxin and a gene encoding a His-tag were further inserted into the gene on the 5′ and 3′ end sides thereof, respectively.

2. Synthesis of dPPR Proteins

mRNAs of the dPPR molecules were obtained by using SP6 RNA Polymerase (Promega). The reaction conditions were determined according to the protocol described in the product information. Proteins of PPRs were obtained by using WEPRO7240H (CellFree Science). The reaction conditions were determined according to the protocol described in the product information.

### 3. DNA-Protein Pull-Down Assay

To each of the modified type rPPRs and crPPR (7L/31F), bovine thymus double-stranded DNA cellulose beads (Sigma-Aldrich, 2 mg), and a buffer (20 mM HEPES-KOH, pH 7.9, 60 mM NaCl, 12.5 mM MgCl₂, 0.3% Triton X-100) were added, and the reaction was allowed at 4° C. for 1 hour. The beads were washed 3 times with a washing solution (10 mM Tris-HCl, pH 8.0, 300 mM NaCl, 0.3% Triton X-100), a 5×SDS-PAGE sample buffer was added to them, and they were heat-treated at 95° C. for 5 minutes to perform elution.

### 4. Western Blotting

Each protein was separated by using 5 to 20% acrylamide gel (Wako Pure Chemical Industries), and transferred to a nitrocellulose membrane. As the transfer buffer, AquaBlot High Efficiency Transfer Buffer (Wako Pure Chemical Industries) was used. Blocking was performed with a 5% skim milk solution, and then the reaction was allowed with 1 μg/ml of HRP-labeled anti-His-tag antibody (Wako Pure Chemical Industries) at room temperature for 1 hour. For the detection, Immunostar Zeta (Wako Pure Chemical Industries) was used. For the detection of the chemiluminescence, Amersham Imager 600 (GE Healthcare) and LAS-4000 (Fuji Photo Film) were used.

### RESULTS AND DISCUSSION

The DNA-binding power was represented with a value obtained by standardization in which luminescence intensity of each pulled-down protein was divided with luminescence intensity at input 3%. Comparison of the DNA-binding powers of the modified type rPPRs and CrPPR (7L/31F) was performed by t-test at 5% significance level (p<0.06). As a result, significant differences were observed for the modified type rPPRs introduced with A.A. 9A, A.A. 18K, A.A. 31I, A.A. 32K, and A.A. 9A/10Y (FIG. 3). These results revealed that a DNA-binding ability can be imparted to PPR by introducing these amino acid sequences.

The sequences of crPPR (7L/31F) and the modified type PPR motifs prepared in this example are shown in the following tables.

TABLE 5-1

| | Motif NO. | Sequence | SEQ ID NO.: | Full Length Sequence | SEQ ID NO.: |
|---|---|---|---|---|---|
| crPPR | N terminal side | MGNS | 309 | MGNSVTYTTLISGLGKAGRLEEALELFEEMKE | |
| | 1 | VTYTTLISGLGKAGRLEEALELFEEMKEKGIVPNV | 284 | KGIVPNVVTYTTLISGLGKAGRLEEALELFEE | |
| | 2 | VTYTTLISGLGKAGRLEEALELFEEMKEKGIVPNV | | MKEKGIVPNVVTYTTLISGLGKAGRLEEALEL | |
| | 3 | VTYTTLISGLGKAGRLEEALELFEEMKEKGIVPNV | | FEEMKEKGIVPNVVTYTTLISGLGKAGRLEEA | |
| | 4 | VTYTTLISGLGKAGRLEEALELFEEMKEKGIVPNV | | LELFEEMKEKGIVPNVVTYTTLISGLGKAGAL | |
| | 5 | VTYTTLISGLGKAGRLEEALELFEEMKEKGIVPNV | | EEALELFEEMKEKGIVPNVVTYTTLISGLGKA | |
| | 6 | VTYTTLISGLGKAGRLEEALELFEEMKEKGIVPNV | | GRLEEALELFEEMKEKGIVPNVVTYTTLISGL | |
| | 7 | VTYTTLISGLGKAGRLEEALELFEEMKEKGIVPNV | | GKAGRLEEALELFEEMKEKGIVPNVVTYTTLI | |
| | 8 | VTYTTLISGLGKAGRLEEALELFEEMKEKGIVPNV | | SGLGKAGRLEEALELFEEMKEKGIVPNVVTYT | |
| | C terminal side | VTYTTLISGLGKAG | 310 | TLISGLGKAG | 335 |
| crPPR | N terminal side | MGNS | 309 | MGNSVTYTTLLSGLGKAGRLEEALELFEEMKE | |
| (7L/31F) | 1 | VTYTTLLSGLGKAGRLEEALELFEEMKEKGFVPNV | 311 | KGFVPNVVTYTTLLSGLGKAGRLEEALELFEE | |
| | 2 | VTYTTLLSGLGKAGRLEEALELFEEMKEKGFVPNV | | MKEKGFVPNVVTYTTLLSGLGKAGRLEEALEL | |
| | 3 | VTYTTLLSGLGKAGRLEEALELFEEMKEKGFVPNV | | FEEMKEKGFVPNVVTYTTLLSGLGKAGRLEEA | |
| | 4 | VTYTTLLSGLGKAGRLEEALELFEEMKEKGFVPNV | | LELFEEMKEKGFVPNVVTYTTLLSGLGKAGRL | |
| | 5 | VTYTTLLSGLGKAGRLEEALELFEEMKEKGFVPNV | | EEALELFEEMKEKGFVPNVVTYTTLLSGLGKA | |
| | 6 | VTYTTLLSGLGKAGRLEEALELFEEMKEKGFVPNV | | GRLEEALELFEEMKEKGFVPNVVTYTTLLSGL | |
| | 7 | VTYTTLLSGLGKAGRLEEALELFEEMKEKGFVPNV | | GKAGRLEEALELF | |
| | 8 | VTYTTLLSGLGKAGRLEEALELFEEMKEKGFVPNV | | EEMKEKGFVPNVVTYTTLLSGLGKAGRLEEAL | |
| | C terminal side | VTYTTLLSGLGKAG | 312 | ELFEEMKEKGFVPNVVTYTTLLSGLGKAG | 336 |
| 71 | N terminal side | MGNS | 309 | MGNSVTYTTLISGLGKAGRLEEALELFEEMKE | |
| | 1 | VTYTTLISGLGKAGRLEEALELFEEMKEKGFVPNV | 313 | KGFVPNVVTYTTLISGLGKAGRLEEALELFEE | |
| | 2 | VTYTTLIGLGKAGRLEEALELFEEMKEKGFVPNV | | MKEKGFVPNVVTYTTLISGLGKAGRLEEALEL | |
| | 3 | VTYTTLISGLGKAGRLEEALELFEEMKEKGFVPNV | | FEEMKEKGFVPNVVTYTTLISGLGKAGRLEEA | |
| | 4 | VTYTTLIGLGKAGRLEEALELFEEMKEKGFVPNV | | LELFEEMKEKGFVPNVVTYTTLISGLGKAGRL | |
| | 5 | VTYTTLISGLGKAGRLEEALELFEEMKEKGFVPNV | | EEALELFEEMKEKGFVPNVVTYTTLISGLGKA | |
| | 6 | VTYTTLISGLGKAGRLEEALELFEEMKEKGFVPNV | | GRLEEALELFEEMKEKGFVPNVVTYTTLISGL | |
| | 7 | VTYTTLISGLGKAGRLEEALELFEEMKEKGFVPNV | | GKAGRLEEALELFEEMKEKGFVPNVVTYTTLI | |
| | 8 | VTYTTLISGLGKAGRLEEALELFEEMKEKGFVPNV | | SGLGKAGRLEEALELFEEMKEKGFVPNVVTYT | |
| | C terminal side | VTYTTLISGLGKAG | 310 | TLISGLGKAG | 337 |
| 9A | N terminal side | MGNS | 309 | MGNSVTYTTLLSALGKAGRLEEALELFEEMKE | |
| | 1 | VTYTTLLSALGKAGRLEEALELFEEMKEKGFVPNV | 314 | KGFVPNVVTYTTLLSALGKAGRLEEALELFEE | |
| | 2 | VTYTTLLSALGKAGRLEEALELFEEMKEKGFVPNV | | MKEKGFVPNVVTYTTLLSALGKAGRLEEALEL | |
| | 3 | VTYTTLLSALGKAGRLEEALELFEEMKEKGFVPNV | | FEEMKEKGFVPNVVTYTTLLSALGKAGRLEEA | |
| | 4 | VTYTTLLSALGKAGRLEEALELFEEMKEKGFVPNV | | LELFEEMKEKGFVPNVVTYTTLLSALGKAGRL | |
| | 5 | VTYTTLLSALGKAGRLEEALELFEEMKEKGFVPNV | | EEALELFEEMKEKGFVPNVVTYTTLLSALGKA | |
| | 6 | VTYTTLLSALGKAGRLEEALELFEEMKEKGFVPNV | | GRLEEALELFEEMKEKGFVPNVVTYTTLLSAL | |
| | 7 | VTYTTLLSALGKAGRLEEALELFEEMKEKGFVPNV | | GKAGRLEEALELFEEMKEKGFVPNVVTYTTLL | |
| | 8 | VTYTTLLSALGKAGRLEEALELFEEMKEKGFVPNV | | SALGKAGRLEEALELFEEMKEKGFVPNVVTYT | |
| | C terminal side | VTYTTLLSALGKAG | 315 | TLLSALGKAG | 338 |
| 10Y | N terminal side | MGNS | 309 | MGNSVTYTTLLSGYGKAGRLEEALELFEEMKE | |
| | 1 | VTYTTLLSGYGKAGRLEEALELFEEMKEKGFVPNV | 316 | KGFVPNVVTYTTLLSGYGKAGRLEEALELFEE | |
| | 2 | VTYTTLLSGYGKAGRLEEALELFEEMKEKGFVPNV | | MKEKGFVPNVVTYTTLLSGYGKAGRLEEALEL | |
| | 3 | VTYTTLLSGYGKAGRLEEALELFEEMKEKGFVPNV | | FEEMKEKGFVPNVVTYTTLLSGYGKAGRLEEA | |
| | 4 | VTYTTLLSGYGKAGRLEEALELFEEMKEKGFVPNV | | LELFEEMKEKGFVPNVVTYTTLLSGYGKAGRL | |
| | 5 | VTYTTLLSGYGKAGRLEEALELFEEMKEKGFVPNV | | EEALELFEEMKEKGFVPNVVTYTTLLSGYGKA | |
| | 6 | VTYTTLLSGYGKAGRLEEALELFEEMKEKGFVPNV | | GRLEEALELFEEMKEKGFVPNVVTYTTLLSGY | |
| | 7 | VTYTTLLSGYGKAGRLEEALELFEEMKEKGFVPNV | | GKAGRLEEALELFEEMKEKGFVPNVVTYTTLL | |
| | 8 | VTYTTLLSGYGKAGRLEEALELFEEMKEKGFVPNV | | SGYGKAGRLEEALELFEEMKEKGFVPNVVTYT | |
| | C terminal side | VTYTTLLSGYGKAG | 317 | TLLSGYGKAG | 339 |
| 18K | N terminal side | MGNS | 309 | MGNSVTYTTLLSGLGKAGRLEKALELFEEMKE | |
| | 1 | VTYTTLLSGLGKAGRLEKALELFEEMKEKGFVPNV | 318 | KGFVPNVVTYTTLLSGLGKAGRLEKALELFEE | |
| | 2 | VTYTTLLSGLGKAGRLEKALELFEEMKEKGFVPNV | | MKEKGFVPNVVTYTTLLSGLGKAGRLEKALEL | |
| | 3 | VTYTTLLSGLGKAGRLEKALELFEEMKEKGFVPNV | | FEEMKEKGFVPNVVTYTTLLSGLGKAGRLEKA | |
| | 4 | VTYTTLLSGLGKAGRLEKALELFEEMKEKGFVPNV | | LELFEEMKEKGFVPNVVTYTTLLSGLGKAGRL | |
| | 5 | VTYTTLLSGLGKAGRLEKALELFEEMKEKGFVPNV | | EKALELFEEMKEKGFVPNVVTYTTLLSGLGKA | |
| | 6 | VTYTTLLSGLGKAGRLEKALELFEEMKEKGFVPNV | | GRLEKALELFEEMKEKGFVPNVVTYTTLLSGL | |
| | 7 | VTYTTLLSGLGKAGRLEKALELFEEMKEKGFVPNV | | GKAGRLEKALELFEEMKEKGFVPNVVTYTTLL | |
| | 8 | VTYTTLLSGLGKAGRLEKALELFEEMKEKGFVPNV | | SGLGKAGRLEKALELFEEMKEKGFVPNVVTYT | |
| | C terminal side | VTYTTLLSGLGKAG | 312 | TLLSGLGKAG | 340 |

TABLE 5-2

| | Motif NO. | Sequence | SEQ ID NO.: | Full Length Sequence | SEQ ID NO.: |
|---|---|---|---|---|---|
| 20E | N terminal side | MGNS | 309 | MGNSVTYTTLLSGLGKAGRLEEAEELFEEMKE | |
| | 1 | VTYTTLLSGLGKAGRLEEAEELFEEMKEKGFVPNV | 319 | KGFVPNVVTYTTLLSGLGKAGRLEEAEELFEE | |
| | 2 | VTYTTLLSGLGKAGRLEEAEELFEEMKEKGFVPNV | | MKEKGFVPNVVTYTTLLSGLGKAGRLEEAEEL | |
| | 3 | VTYTTLLSGLGKAGRLEEAEELFEEMKEKGFVPNV | | FEEMKEKGFVPNVVTYTTLLSGLGKAGRLEEA | |
| | 4 | VTYTTLLSGLGKAGRLEEAEELFEEMKEKGFVPNV | | EELFEEMKEKGFVPNVVTYTTLLSGLGKAGRL | |
| | 5 | VTYTTLLSGLGKAGRLEEAEELFEEMKEKGFVPNV | | EEAEELFEEMKEKGFVPNVVTYTTLLSGLGKA | |
| | 6 | VTYTTLLSGLGKAGRLEEARELFEEMKEKGFVPNV | | GRLEEAEELFEEMKEKGFVPNVVTYTTLLSGL | |
| | 7 | VTYTTLLSGLGKAGRLEEARELFEEMKEKGFVPNV | | GKAGRLEEAEELFEEMKEKGFVPNVVTYTTLL | |
| | 8 | VTYTTLLSGLGKAGRLEEAEELFEEMKEKGFVPNV | | SGLGKAGRLEEAEELFEEMKEKGFVPNVVTYT | |
| | C terminal side | VTYTTLLSGLGKAG | 312 | TLLSGLGKAG | 341 |
| 29E | N terminal side | MGNS | 309 | MGNSVTYTTLLSGLGKAGRLEEALELFEEMKE | |
| | 1 | VTYTTLLSGLGKAGRLEEALELFEEMKEEGFVPNV | 320 | EGFVPNVVTYTTLLSGLGKAGRLEEALELFEE | |
| | 2 | VTYTTLLSGLGKAGRLEEALELFEEMKEEGFVPNV | | MKEEGFVPNVVTYTTLLSGLGKAGRLEEALEL | |
| | 3 | VTYTTLLSGLGKAGRLEEALELFEEMKEEGFVPNV | | FEEMKEEGFVPNVVTYTTLLSGLGKAGRLEEA | |
| | 4 | VTYTTLLSGLGKAGRLEEALELFEEMKEEGFVPNV | | LELFEEMKEEGFVPNVVTYTTLLSGLGKAGRL | |
| | 5 | VTYTTLLSGLGKAGRLEEALELFEEMKEEGFVPNV | | EEALELFEEMKEEGFVPNVVTYTTLLSGLGKA | |
| | 6 | VTYTTLLSGLGKAGRLEEALELFEEMKEEGFVPNV | | GRLEEALELFEEMKEEGFVPNVVTYTTLLSGL | |
| | 7 | VTYTTLLSGLGKAGRLEEALELFEEMKEEGFVPNV | | GKAGRLEEALELFEEMKEEGFVPNVVTYTTLL | |
| | 8 | VTYTTLLSGLGKAGRLEEALELFEEMKEEGFVPNV | | SGLGKAGRLEEALELFEEMKEEGFVPNVVTYT | |
| | C terminal side | VTYTTLLSGLGKAG | 312 | TLLSGLGKAG | 342 |
| 31I | N terminal side | MGNS | 309 | MGNSVTYTTLLSGLGKAGRLEEALELFEEMKE | |
| | 1 | VTYTTLLSGLGKAGRLEEALELFEEMKEKGIVPNV | 321 | KGIVPNVVTYTTLLSGLGKAGRLEEALELFEE | |
| | 2 | VTYTTLLSGLGKAGRLEEALELFEEMKEKGIVPNV | | MKEKGIVPNVVTYTTLLSGLGKAGRLEEALEL | |
| | 3 | VTYTTLLSGLGKAGRLEEALELFEEMKEKGIVPNV | | FEEMKEKGIVPNVVTYTTLLSGLGKAGRLEEA | |
| | 4 | VTYTTLLSGLGKAGRLEEALELFEEMKEKGIVPNV | | LELFEEMKEKGIVPNVVTYTTLLSGLGKAGAL | |
| | 5 | VTYTTLLSGLGKAGRLEEALELFEEMKEKGIVPNV | | EEALELFEEMKEKGIVPNVVTYTTLLSGLGKA | |
| | 6 | VTYTTLLSGLGKAGRLEEALELFEEMKEKGIVPNV | | GRLEEALELFEEMKEKGIVPNVVTYTTLLSGL | |
| | 7 | VTYTTLLSGLGKAGRLEEALELFEEMKEKGIVPNV | | GKAGRLEEALELFEEMKEKGIVPNVVTYTTLL | |
| | 8 | VTYTTLLSGLGKAGRLEEALELFEEMKEKGIVPNV | | SGLGKAGRLEEALELFEEMKEKGIVPNVVTYT | |
| | C terminal side | VTYTTLLSGLGKAG | 312 | TLLSGLGKAG | 343 |
| 32K | N terminal side | MGNS | 309 | MGNSVTYTTLLSGLGKAGRLEEALELFEEMKE | |
| | 1 | VTYTTLLSGLGKAGRLEEALELFEEMKEKGFKPNV | 322 | KGFKPNVVTYTTLLSGLGKAGRLEEALELFEE | |
| | 2 | VTYTTLLSGLGKAGRLEEALELFEEMKEKGFKPNV | | MKEKGFKPNVVTYTTLLSGLGKAGRLEEALEL | |
| | 3 | VTYTTLLSGLGKAGRLEEALELFEEMKEKGFKPNV | | FEEMKEKGFKPNVVTYTTLLSGLGKAGRLEEA | |
| | 4 | VTYTTLLSGLGKAGRLEEALELFEEMKEKGFKPNV | | LELFEEMKEKGFKPNVVTYTTLLSGLGKAGAL | |
| | 5 | VTYTTLLSGLGKAGRLEEALELFEEMKEKGFKPNV | | EEALELFEEMKEKGFKPNVVTYTTLLSGLGKA | |
| | 6 | VTYTTLLSGLGKAGRLEEALELFEEMKEKGFKPNV | | GRLEEALELFEEMKEKGFKPNVVTYTTLLSGL | |
| | 7 | VTYTTLLSGLGKAGRLEEALELFEEMKEKGFKPNV | | GKAGRLEEALELFEEMKEKGFKPNVVTYTTLL | |
| | 8 | VTYTTLLSGLGKAGRLEEALELFEEMKEKGFKPNV | | SGLGKAGRLEEALELFEEMKEKGFKPNVVTYT | |
| | C terminal side | VTYTTLLSGLGKAG | 312 | TLLSGLGKAG | 344 |
| 9A/10Y | N terminal side | MGNS | 309 | MGNSVTYTTLLSAYGKAGRLEEALELFEEMKE | |
| | 1 | VTYTTLLSAYGKAGRLEEALELFEEMKEKGFVPNV | 323 | KGFVPNVVTYTTLLSAYGKAGRLEEALELFEE | |
| | 2 | VTYTTLLSAYGKAGRLEEALELFEEMKEKGFVPNV | | MKEKGFVPNVVTYTTLLSAYGKAGRLEEALEL | |
| | 3 | VTYTTLLSAYGKAGRLEEALELFEEMKEKGFVPNV | | FEEMKEKGFVPNVVTYTTLLSAYGKAGRLEEA | |
| | 4 | VTYTTLLSAYGKAGRLEEALELFEEMKEKGFVPNV | | LELFEEMKEKGFVPNVVTYTTLLSAYGKAGRL | |
| | 5 | VTYTTLLSAYGKAGRLEEALELFEEMKEKGFVPNV | | EEALELFEEMKEKGFVPNVVTYTTLLSAYGKA | |
| | 6 | VTYTTLLSAYGKAGRLEEALELFEEMKEKGFVPNV | | GRLEEALELFEEMKEKGFVPNVVTYTTLLSAY | |
| | 7 | VTYTTLLSAYGKAGRLEEALELFEEMKEKGFVPNV | | GKAGRLEEALELFEEMKEKGFVPNVVTYTTLL | |
| | 8 | VTYTTLLSAYGKAGRLEEALELFEEMKEKGFVPNV | | SAYGKAGRLEEALELFEEMKEKGFVPNVVTYT | |
| | C terminal side | VTYTTLLSAYGKAG | 324 | TLLSAYGKAG | 345 |

### Example 4

### Evaluation of Amino Acids Having Similar Characteristics

It was examined whether the effect would also be obtained even when amino acids having similar characteristics are used for A.A. 18K, A.A. 31I, A.A. 32K, and A.A.9A/10Y. In this experiment, there were used histidine (H) and arginine (R), which are basic amino acids like K, for No. 18 A.A. and No. 32 A.A., valine (V) and leucine (L), which have a branched chain like I, for No. 31 A.A., and phenylalanine (F) and tryptophan (W), which have an aromatic group like Y, for No. 10 A.A. The DNA-binding ability was evaluated by analysis performed in the same manner as that used in Example 3.

### RESULTS AND DISCUSSION

The DNA-binding powers of the modified type rPPRs and crPPR (7L/31F) were compared by t-test at a significance level of 5% (p<0.06). As a result, significant difference was observed for all the modified type rPPRs (FIG. 4). These results revealed that even when amino acids having similar characteristics are used, a DNA-binding ability can be imparted.

The sequences of the modified type rPPR motifs prepared in this example are shown in the following table.

TABLE 6

| | Motif NO. | Sequence | SEQ ID NO.: | Full Length Sequence | SEQ ID NO.: |
|---|---|---|---|---|---|
| 18H | N terminal side | MGNS | 309 | MGNSVTYTTLLSGLGKAGRLEHALELFEEMKE | |
| | 1 | VTYTTLLSGLGKAGRLEHALELFEEMKEKGFVPNV | 325 | KGFVPNVVTYTTLLSGLGKAGRLEHALELFEE | |
| | 2 | VTYTTLLSGLGKAGRLEHALELFEEMKEKGFVPNV | | MKEKGFVPNVVTYTTLLSGLGKAGRLEHALEL | |
| | 3 | VTYTTLLSGLGKAGRLEHALELFEEMKEKGFVPNV | | FEEMKEKGFVPNVVTYTTLLSGLGKAGRLEHA | |
| | 4 | VTYTTLLSGLGKAGRLEHALELFEEMKEKGFVPNV | | LELFEEMKEKGFVPNVVTYTTLLSGLGKAGRL | |
| | 5 | VTYTTLLSGLGKAGRLEHALELFEEMKEKGFVPNV | | EHALELFEEMKEKGFVPNVVTYTTLLSGLGKA | |
| | 6 | VTYTTLLSGLGKAGRLEHALELFEEMKEKGFVPNV | | GRLEHALELFEEMKEKGFVPNVVTYTTLLSGL | |
| | 7 | VTYTTLLSGLGKAGRLEHALELFEEMKEKGFVPNV | | GKAGRLEHALELFEEMKEKGFVPNVVTYTTLL | |
| | 8 | VTYTTLLSGLGKAGRLEHALELFEEMKEKGFVPNV | | SGLGKAGRLEHALELFEEMKEKGFVPNVVTYT | |
| | C terminal sideV | TYTTLLSGLGKAG | 312 | TLLSGLGKAG | 346 |
| 18R | N terminal side | MGNS | 309 | MGNSVTYTTLLSGLGKAGRLERALELFEEMKE | |
| | 1 | VTYTTLLSGLGKAGRLERALELFEEMKEKGFVPNV | 326 | KGFVPNVVTYTTLLSGLGKAGRLERALELFEE | |
| | 2 | VTYTTLLSGLGKAGRLERALELFEEMKEKGFVPNV | | MKEKGFVPNVVTYTTLLSGLGKAGRLERALEL | |
| | 3 | VTYTTLLSGLGKAGRLERALELFEEMKEKGFVPNV | | FEEMKEKGFVPNVVTYTTLLSGLGKAGRLERA | |
| | 4 | VTYTTLLSGLGKAGRLERALELFEEMKEKGFVPNV | | LELFEEMKEKGFVPNVVTYTTLLSGLGKAGRL | |
| | 5 | VTYTTLLSGLGKAGRLERALELFEEMKEKGFVPNV | | ERALELFEEMKEKGFVPNVVTYTTLLSGLGKA | |
| | 6 | VTYTTLLSGLGKAGRLERALELFEEMKEKGFVPNV | | GRLERALELFEEMKEKGFVPNVVTYTTLLSGL | |
| | 7 | VTYTTLLSGLGKAGRLERALELFEEMKEKGFVPNV | | GKAGRLERALELFEEMKEKGFVPNVVTYTTLL | |
| | 8 | VTYTTLLSGLGKAGRLERALELFEEMKEKGFVPNV | | SGLGKAGRLERALELFEEMKEKGFVPNVVTYT | |
| | C terminal side | VTYTTLLSGLGKAG | 312 | TLLSGLGKAG | 347 |
| 31V | N terminal side | MGNS | 309 | MGNSVTYTTLLSGLGKAGRLEKALELFEEMKE | |
| | 1 | VTYTTLLSGLGKAGRLEKALELFEEMKEKGVVPNV | 327 | KGVVPNVVTYTTLLSGLGKAGRLEKALELFEE | |
| | 2 | VTYTTLLSGLGKAGRLEKALELFEEMKEKGVVPNV | | MKEKGVVPNVVTYTTLLSGLGKAGRLEKALEL | |
| | 3 | VTYTTLLSGLGKAGRLEKALELFEEMKEKGVVPNV | | FEEMKEKGVVPNVVTYTTLLSGLGKAGRLEKA | |
| | 4 | VTYTTLLSGLGKAGRLEKALELFEEMKEKGVVPNV | | LELFEEMKEKGVVPNVVTYTTLLSGLGKAGAL | |
| | 5 | VTYTTLLSGLGKAGRLEKALELFEEMKEKGVVPNV | | EKALELFEEMKEKGVVPNVVTYTTLLSGLGKA | |
| | 6 | VTYTTLLSGLGKAGRLEKALELFEEMKEKGVVPNV | | GRLEKALELFEEMKEKGVVPNVVTYTTLLSGL | |
| | 7 | VTYTTLLSGLGKAGRLEKALELFEEMKEKGVVPNV | | GKAGRLEKALELFEEMKEKGVVPNVVTYTTLL | |
| | 8 | VTYTTLLSGLGKAGRLEKALELFEEMKEKGVVPNV | | SGLGKAGRLEKALELFEEMKEKGVVPNVVTYT | |
| | C terminal side | VTYTTLLSGLGKAG | 312 | TLLSGLGKAG | 348 |
| 31L | N terminal side | MGNS | 309 | MGNSVTYTTLLSGLGKAGRLEKALELFEEMKE | |
| | 1 | VTYTTLLSGLGKAGRLEKALELFEEMKEKGLVPNV | 328 | KGLVPNVVTYTTLLSGLGKAGRLEKALELFEE | |
| | 2 | VTYTTLLSGLGKAGRLEKALELFEEMKEKGLVPNV | | MKEKGLVPNVVTYTTLLSGLGKAGRLEKALEL | |
| | 3 | VTYTTLLSGLGKAGRLEKALELFEEMKEKGLVPNV | | FEEMKEKGLVPNVVTYTTLLSGLGKAGRLEKA | |
| | 4 | VTYTTLLSGLGKAGRLEKALELFEEMKEKGLVPNV | | LELFEEMKEKGLVPNVVTYTTLLSGLGKAGAL | |
| | 5 | VTYTTLLSGLGKAGRLEKALELFEEMKEKGLVPNV | | EKALELFEEMKEKGLVPNVVTYTTLLSGLGKA | |
| | 6 | VTYTTLLSGLGKAGRLEKALELFEEMKEKGLVPNV | | GRLEKALELFEEMKEKGLVPNVVTYTTLLSGL | |
| | 7 | VTYTTLLSGLGKAGRLEKALELFEEMKEKGLVPNV | | GKAGRLEKALELFEEMKEKGLVPNVVTYTTLL | |
| | 8 | VTYTTLLSGLGKAGRLEKALELFEEMKEKGLVPNV | | SGLGKAGRLEKALELFEEMKEKGLVPNVVTYT | |
| | C terminal side | VTYTTLLSGLGKAG | 312 | TLLSGLGKAG | 349 |
| 32H | N terminal side | MGNS | 309 | MGNSVTYTTLLSGLGKAGRLEEALELFEEMKE | |
| | 1 | VTYTTLLSGLGKAGRLEEALELFEEMKEKGFHPNV | 329 | KGFHPNVVTYTTLLSGLGKAGRLEEALELFEE | |
| | 2 | VTYTTLLSGLGKAGRLEEALELFEEMKEKGFHPNV | | MKEKGFHPNVVTYTTLLSGLGKAGRLEEALEL | |
| | 3 | VTYTTLLSGLGKAGRLEEALELFEEMKEKGFHPNV | | FEEMKEKGFHPNVVTYTTLLSGLGKAGRLEEA | |
| | 4 | VTYTTLLSGLGKAGRLEEALELFEEMKEKGFHPNV | | LELFEEMKEKGFHPNVVTYTTLLSGLGKAGAL | |
| | 5 | VTYTTLLSGLGKAGRLEEALELFEEMKEKGFHPNV | | EEALELFEEMKEKGFHPNVVTYTTLLSGLGKA | |
| | 6 | VTYTTLLSGLGKAGRLEEALELFEEMKEKGFHPNV | | GRLEEALELFEEMKEKGFHPNVVTYTTLLSGL | |
| | 7 | VTYTTLLSGLGKAGRLEEALELFEEMKEKGFHPNV | | GKAGRLEEALELFEEMKEKGFHPNVVTYTTLL | |
| | 8 | VTYTTLLSGLGKAGRLEEALELFEEMKEKGFHPNV | | SGLGKAGRLEEALELFEEMKEKGFHPNVVTYT | |
| | C terminal side | VTYTTLLSGLGKAG | 312 | TLLSGLGKAG | 350 |
| 32R | N terminal side | MGNS | 309 | MGNSVTYTTLLSGLGKAGRLEEALELFEEMKE | |
| | 1 | VTYTTLLSGLGKAGRLEEALELFEEMKEKGFRPNV | 330 | KGFRPNVVTYTTLLSGLGKAGRLEEALELFEE | |
| | 2 | VTYTTLLSGLGKAGRLEEALELFEEMKEKGFRPNV | | MKEKGFRPNVVTYTTLLSGLGKAGRLEEALEL | |
| | 3 | VTYTTLLSGLGKAGRLEEALELFEEMKEKGFRPNV | | FEEMKEKGFRPNVVTYTTLLSGLGKAGRLEEA | |
| | 4 | VTYTTLLSGLGKAGRLEEALELFEEMKEKGFRPNV | | LELFEEMKEKGFRPNVVTYTTLLSGLGKAGAL | |
| | 5 | VTYTTLLSGLGKAGRLEEALELFEEMKEKGFRPNV | | EEALELFEEMKEKGFRPNVVTYTTLLSGLGKA | |
| | 6 | VTYTTLLSGLGKAGRLEEALELFEEMKEKGFRPNV | | GRLEEALELFEEMKEKGFRPNVVTYTTLLSGL | |
| | 7 | VTYTTLLSGLGKAGRLEEALELFEEMKEKGFRPNV | | GKAGRLEEALELFEEMKEKGFRPNVVTYTTLL | |
| | 8 | VTYTTLLSGLGKAGRLEEALELFEEMKEKGFRPNV | | SGLGKAGRLEEALELFEEMKEKGFRPNVVTYT | |
| | C terminal side | VTYTTLLSGLGKAG | 312 | TLLSGLGKAG | 351 |
| 9A/10F | N terminal side | MGNS | 309 | MGNSVTYTTLLSAFGKAGRLEEALELFEEMKE | |
| | 1 | VTYTTLLSAFGKAGRLEEALELFEEMKEKGFVPNV | 331 | KGFVPNVVTYTTLLSAFGKAGRLEEALELFEE | |
| | 2 | VTYTTLLSAFGKAGRLEEALELFEEMKEKGFVPNV | | MKEKGFVPNVVTYTTLLSAFGKAGRLEEALEL | |
| | 3 | VTYTTLLSAFGKAGRLEEALELFEEMKEKGFVPNV | | FEEMKEKGFVPNVVTYTTLLSAFGKAGRLEEA | |
| | 4 | VTYTTLLSAFGKAGRLEEALELFEEMKEKGFVPNV | | LELFEEMKEKGFVPNVVTYTTLLSAFGKAGRL | |
| | 5 | VTYTTLLSAFGKAGRLEEALELFEEMKEKGFVPNV | | EEALELFEEMKEKGFVPNVVTYTTLLSAFGKA | |
| | 6 | VTYTTLLSAFGKAGRLEEALELFEEMKEKGFVPNV | | GRLEEALELFEEMKEKGFVPNVVTYTTLLSAF | |
| | 7 | VTYTTLLSAFGKAGRLEEALELFEEMKEKGFVPNV | | GKAGRLEEALELFEEMKEKGFVPNVVTYTTLL | |
| | 8 | VTYTTLLSAFGKAGRLEEALELFEEMKEKGFVPNV | | SAFGKAGRLEEALELFEEMKEKGFVPNVVTYT | |
| | C terminal side | VTYTTLLSAFGKAG | 332 | TLLSAFGKAG | 352 |
| 9A/10W | N terminal side | MGNS | 309 | MGNSVTYTTLLSAWGKAGRLEEALELFEEMKE | |
| | 1 | VTYTTLLSAWGKAGRLEEALELFEEMKEKGFVPNV | 333 | KGFVPNVVTYTTLLSAWGKAGRLEEALELFEE | |
| | 2 | VTYTTLLSAWGKAGRLEEALELFEEMKEKGFVPNV | | MKEKGFVPNVVTYTTLLSAWGKAGRLEEALEL | |
| | 3 | VTYTTLLSAWGKAGRLEEALELFEEMKEKGFVPNV | | FEEMKEKGFVPNVVTYTTLLSAWGKAGRLEEA | |
| | 4 | VTYTTLLSAWGKAGRLEEALELFEEMKEKGFVPNV | | LELFEEMKEKGFVPNVVTYTTLLSAWGKAGRL | |
| | 5 | VTYTTLLSAWGKAGRLEEALELFEEMKEKGFVPNV | | EEALELFEEMKEKGFVPNVVTYTTLLSAWGKA | |
| | 6 | VTYTTLLSAWGKAGRLEEALELFEEMKEKGFVPNV | | GRLEEALELFEEMKEKGFVPNVVTYTTLLSAW | |
| | 7 | VTYTTLLSAWGKAGRLEEALELFEEMKEKGFVPNV | | GKAGRLEEALELFEEMKEKGFVPNVVTYTTLL | |
| | 8 | VTYTTLLSAWGKAGRLEEALELFEEMKEKGFVPNV | | SAWGKAGRLEEALELFEEMKEKGFVPNVVTYT | |
| | C terminal side | VTYTTLLSAWGKAG | 334 | TLLSAWGKAG | 353 |

### Example 5

### Evaluation of Contents of A.A. 9A, A.A. 18K, A.A. 31I, A.A. 32K, and A.A. 9A/10Y Required for DNA-Binding Ability

Contents (ratios) of A.A. 9A, A.A. 18K, A.A. 31I, A.A. 32K, and A.A. 9A/10Y required for imparting a DNA-binding ability were examined. The content (ratio) referred to here is an amount (ratio) of motifs having the aforementioned amino acid sequences in PPR molecule. In this experiment, DNA-binding abilities of modified type rPPRs in which 2 motifs (25% of the whole) or 4 motifs (50% of the whole) of crPPR (7L/31F) on the N-terminus side were motifs having these amino acid sequences were analyzed. The DNA-binding ability was analyzed in the same manner as that used in Example 3.

### RESULTS AND DISCUSSION

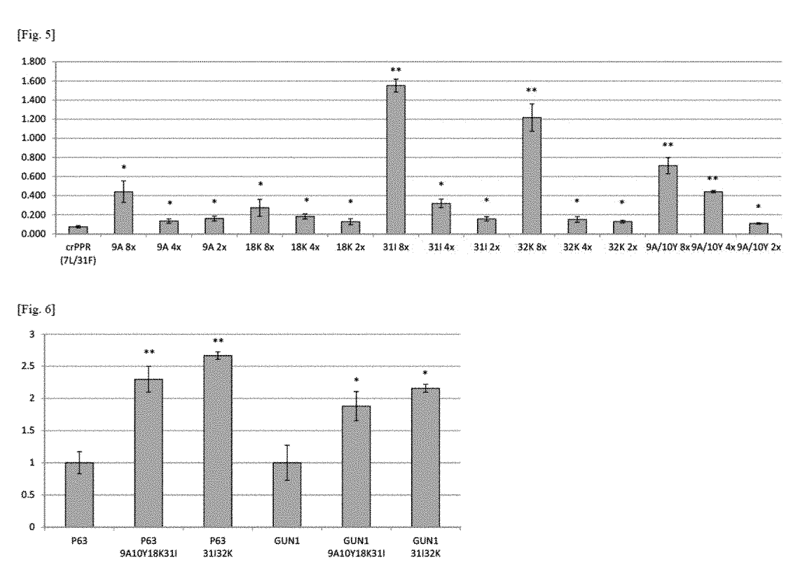

The DNA-binding powers of the modified type rPPRs and crPPR (7L/31F) were compared by t-test at a significance level of 5% (p<0.06). As a result, significant difference was observed for all the modified type rPPRs (FIG. 5). These results revealed that a DNA-binding ability can be imparted with a content of 2 or more (or 25% or more of the whole) of PPR motifs introduced with A.A. 9A, A.A. 18K, A.A. 31I, A.A. 32K, and A.A. 9A/10Y.

The sequences of the modified type rPPR motifs prepared in this example are shown in the following table.

TABLE 7

| | Motif NO. | Sequence | SEQ ID NO.: | Full Length Sequence | SEQ ID NO.: |
|---|---|---|---|---|---|
| 18K 50% | N terminal side | MGNS | 309 | MGNSVTYTTLLSGLGKAGRLEKALELFEEMKE | |
| | 1 | VTYTTLLSGLGKAGRLEKALELFEEMKEKGFVPNV | 318 | KGFVPNVVTYTTLLSGLGKAGRLEKALELFEE | |
| | 2 | VTYTTLLSGLGKAGRLEKALELFEEMKEKGFVPNV | | MKEKGFVPNVVTYTTLLSGLGKAGRLEKALEL | |
| | 3 | VTYTTLLSGLGKAGRLEKALELFEEMKEKGFVPNV | | FEEMKEKGFVPNVVTYTTLLSGLGKAGRLEKA | |
| | 4 | VTYTTLLSGLGKAGRLEKALELFEEMKEKGFVPNV | | LELFEEMKEKGFVPNVVTYTTLLSGLGKAGRL | |
| | 5 | VTYTTLLSGLGKAGRLEEALELFEEMKEKGFVPNV | 311 | EEALELFEEMKEKGFVPNVVTYTTLLSGLGKA | |
| | 6 | VTYTTLLSGLGKAGRLEEALELFEEMKEKGFVPNV | | GRLEEALELFEEMKEKGFVPNVVTYTTLLSGL | |
| | 7 | VTYTTLLSGLGKAGRLEEALELFEEMKEKGFVPNV | | GKAGRLEEALELFEEMKEKGFVPNVVTYTTLL | |
| | 8 | VTYTTLLSGLGKAGRLEEALELFEEMKEKGFVPNV | | SGLGKAGRLEEALELFEEMKEKGFVPNVVTYT | |
| | C terminal side | VTYTTLLSGLGKAG | 312 | TLLSGLGKAG | 354 |
| 18K 25% | N terminal side | MGNS | 309 | MGNSVTYTTLLSGLGKAGRLEKALELFEEMKE | |
| | 1 | VTYTTLLSGLGKAGRLEKALELFEEMKEKGFVPNV | 319 | KGFVPNVVTYTTLLSGLGKAGRLEKALELFEE | |
| | 2 | VTYTTLLSGLGKAGRLEKALELFEEMKEKGFVPNV | | MKEKGFVPNVVTYTTLLSGLGKAGRLEEALEL | |
| | 3 | VTYTTLLSGLGKAGRLEEALELFEEMKEKGFVPNV | 311 | FEEMKEKGFVPNVVTYTTLLSGLGKAGRLEEA | |
| | 4 | VTYTTLLSGLGKAGRLEEALELFEEMKEKGFVPNV | | LELFEEMKEKGFVPNVVTYTTLLSGLGKAGRL | |
| | 5 | VTYTTLLSGLGKAGRLEEALELFEEMKEKGFVPNV | | EEALELFEEMKEKGFVPNVVTYTTLLSGLGKA | |
| | 6 | VTYTTLLSGLGKAGRLEEALELFEEMKEKGFVPNV | | GRLEEALELFEEMKEKGFVPNVVTYTTLLSGL | |
| | 7 | VTYTTLLSGLGKAGRLEEALELFEEMKEKGFVPNV | | GKAGRLEEALELFEEMKEKGFVPNVVTYTTLL | |
| | 8 | VTYTTLLSGLGKAGRLEEALELFEEMKEKGFVPNV | | SGLGKAGRLEEALELFEEMKEKGFVPNVVTYT | |
| | C terminal side | VTYTTLLSGLGKAG | 312 | TLLSGLGKAG | 355 |
| 311 50% | N terminal side | MGNS | 309 | MGNSVTYTTLLSGLGKAGRLEEALELFEEMKE | |
| | 1 | VTYTTLLSGLGKAGRLEEALELFEEMKEKGIVPNV | 321 | KGIVPNVVTYTTLLSGLGKAGRLEEALELFEE | |
| | 2 | VTYTTLLSGLGKAGRLEEALELFEEMKEKGIVPNV | | MKEKGIVPNVVTYTTLLSGLGKAGRLEEALEL | |
| | 3 | VTYTTLLSGLGKAGRLEEALELFEEMKEKGIVPNV | | FEEMKEKGIVPNVVTYTTLLSGLGKAGRLEEA | |
| | 4 | VTYTTLLSGLGKAGRLEEALELFEEMKEKGIVPNV | | LELFEEMKEKGIVPNVVTYTTLLSGLGKAGRL | |
| | 5 | VTYTTLLSGLGKAGRLEEALELFEEMKEKGFVPNV | 311 | EEALELFEEMKEKGFVPNVVTYTTLLSGLGKA | |
| | 6 | VTYTTLLSGLGKAGRLEEALELFEEMKEKGFVPNV | | GRLEEALELFEEMKEKGFVPNVVTYTTLLSGL | |
| | 7 | VTYTTLLSGLGKAGRLEEALELFEEMKEKGFVPNV | | GKAGRLEEALELFEEMKEKGFVPNVVTYTTLL | |
| | 8 | VTYTTLLSGLGKAGRLEEALELFEEMKEKGFVPNV | | SGLGKAGRLEEALELFEEMKEKGFVPNVVTYT | |
| | C terminal side | VTYTTLLSGLGKAG | 312 | TLLSGLGKAG | 356 |
| 311 25% | N terminal side | MGNS | 309 | MGNSVTYTTLLSGLGKAGRLEEALELFEEMKE | |
| | 1 | VTYTTLLSGLGKAGRLEEALELFEEMKEKGIVPNV | 321 | KGIVPNVVTYTTLLSGLGKAGRLEEALELFEE | |
| | 2 | VTYTTLLSGLGKAGRLEEALELFEEMKEKGIVPNV | | MKEKGIVPNVVTYTTLLSGLGKAGRLEEALEL | |
| | 3 | VTYTTLLSGLGKAGRLEEALELFEEMKEKGFVPNV | 311 | FEEMKEKGFVPNVVTYTTLLSGLGKAGRLEEA | |
| | 4 | VTYTTLLSGLGKAGRLEEALELFEEMKEKGFVPNV | | LELFEEMKEKGFVPNVVTYTTLLSGLGKAGRL | |
| | 5 | VTYTTLLSGLGKAGRLEEALELFEEMKEKGFVPNV | | EEALELFEEMKEKGFVPNVVTYTTLLSGLGKA | |
| | 6 | VTYTTLLSGLGKAGRLEEALELFEEMKEKGFVPNV | | GRLEEALELFEEMKEKGFVPNVVTYTTLLSGL | |
| | 7 | VTYTTLLSGLGKAGRLEEALELFEEMKEKGFVPNV | | GKAGRLEEALELFEEMKEKGFVPNVVTYTTLL | |
| | 8 | VTYTTLLSGLGKAGRLEEALELFEEMKEKGFVPNV | | SGLGKAGRLEEALELFEEMKEKGFVPNVVTYT | |
| | C terminal side | VTYTTLLSGLGKAG | 312 | TLLSGLGKAG | 357 |
| 32K 50% | N terminal side | MGNS | 309 | MGNSVTYTTLLSGLGKAGRLEEALELFEEMKE | |
| | 1 | VTYTTLLSGLGKAGRLEEALELFEEMKEKGFKPNV | 322 | KGFKPNVVTYTTLLSGLGKAGRLEEALELFEE | |
| | 2 | VTYTTLLSGLGKAGRLEEALELFEEMKEKGFKPNV | | MKEKGFKPNVVTYTTLLSGLGKAGRLEEALEL | |
| | 3 | VTYTTLLSGLGKAGRLEEALELFEEMKEKGFKPNV | | FEEMKEKGFKPNVVTYTTLLSGLGKAGRLEEA | |
| | 4 | VTYTTLLSGLGKAGRLEEALELFEEMKEKGFKPNV | | LELFEEMKEKGFKPNVVTYTTLLSGLGKAGRL | |
| | 5 | VTYTTLLSGLGKAGRLEEALELFEEMKEKGFVPNV | 311 | EEALELFEEMKEKGFVPNVVTYTTLLSGLGKA | |
| | 6 | VTYTTLLSGLGKAGRLEEALELFEEMKEKGFVPNV | | GRLEEALELFEEMKEKGFVPNVVTYTTLLSGL | |
| | 7 | VTYTTLLSGLGKAGRLEEALELFEEMKEKGFVPNV | | GKAGRLEEALELFEEMKEKGFVPNVVTYTTLL | |
| | 8 | VTYTTLLSGLGKAGRLEEALELFEEMKEKGFVPNV | | SGLGKAGRLEEALELFEEMKEKGFVPNVVTYT | |
| | C terminal side | VTYTTLLSGLGKAG | 312 | TLLSGLGKAG | 358 |
| 32K 25% | N terminal side | MGNS | 309 | MGNSVTYTTLLSGLGKAGRLEEALELFEEMKE | |
| | 1 | VTYTTLLSGLGKAGRLEEALELFEEMKEKGFKPNV | 322 | KGFKPNVVTYTTLLSGLGKAGRLEEALELFEE | |
| | 2 | VTYTTLLSGLGKAGRLEEALELFEEMKEKGFKPNV | | MKEKGFKPNVVTYTTLLSGLGKAGRLEEALEL | |
| | 3 | VTYTTLLSGLGKAGRLEEALELFEEMKEKGFVPNV | 311 | FEEMKEKGFVPNVVTYTTLLSGLGKAGRLEEA | |
| | 4 | VTYTTLLSGLGKAGRLEEALELFEEMKEKGFVPNV | | LELFEEMKEKGFVPNVVTYTTLLSGLGKAGRL | |
| | 5 | VTYTTLLSGLGKAGRLEEALELFEEMKEKGFVPNV | | EEALELFEEMKEKGFVPNVVTYTTLLSGLGKA | |
| | 6 | VTYTTLLSGLGKAGRLEEALELFEEMKEKGFVPNV | | GRLEEALELFEEMKEKGFVPNVVTYTTLLSGL | |
| | 7 | VTYTTLLSGLGKAGRLEEALELFEEMKEKGFVPNV | | GKAGRLEEALELFEEMKEKGFVPNVVTYTTLL | |
| | 8 | VTYTTLLSGLGKAGRLEEALELFEEMKEKGFVPNV | | SGLGKAGRLEEALELFEEMKEKGFVPNVVTYT | |
| | C terminal side | VTYTTLLSGLGKAG | 312 | TLLSGLGKAG | 359 |
| 9A/10Y 50% | N terminal side | MGNS | 309 | MGNSVTYTTLLSAYGKAGRLEEALELFEEMKE | |
| | 1 | VTYTTLLSAYGKAGRLEEALELFEEMKEKGFVPNV | 323 | KGFVPNVVTYTTLLSAYGKAGRLEEALELFEE | |
| | 2 | VTYTTLLSAYGKAGRLEEALELFEEMKEKGFVPNV | | MKEKGFVPNVVTYTTLLSAYGKAGRLEEALEL | |
| | 3 | VTYTTLLSAYGKAGRLEEALELFEEMKEKGFVPNV | | FEEMKEKGFVPNVVTYTTLLSAYGKAGRLEEA | |
| | 4 | VTYTTLLSAIGKAGRLEEALELFEEMKEKGFVPNV | | LELFEEMKEKGFVPNVVTYTTLLSGLGKAGRL | |
| | 5 | VTYTTLLSGLGKAGRLEEALELFEEMKEKGFVPNV | 311 | EEALELFEEMKEKGFVPNVVTYTTLLSGLGKA | |
| | 6 | VTYTTLLSGLGKAGRLEEALELFEEMKEKGFVPNV | | GRLEEALELFEEMKEKGFVPNVVTYTTLLSGL | |
| | 7 | VTYTTLLSGLGKAGRLEEALELFEEMKEKGFVPNV | | GKAGRLEEALELFEEMKEKGFVPNVVTYTTLL | |
| | 8 | VTYTTLLSGLGKAGRLEEALELFEEMKEKGFVPNV | | SGLGKAGRLEEALELFEEMKEKGFVPNVVTYT | |
| | C terminal side | VTYTTLLSGLGKAG | 312 | TLLSGLGKAG | 360 |
| 9A/10Y 25% | N terminal side | MGNS | 309 | MGNSVTYTTLLSAYGKAGRLEEALELFEEMKE | |
| | 1 | VTYTTLLSAYGKAGRLEEALELFEEMKEKGFVPNV | 323 | KGFVPNVVTYTTLLSAYGKAGRLEEALELFEE | |
| | 2 | VTYTTLLSAYGKAGRLEEALELFEEMKEKGFVPNV | | MKEKGFVPNVVTYTTLLSGLGKAGRLEEALEL | |
| | 3 | VTYTTLLSGLGKAGRLEEALELFEEMKEKGFVPNV | 311 | FEEMKEKGFVPNVVTYTTLLSGLGKAGRLEEA | |
| | 4 | VTYTTLLSGLGKAGRLEEALELFEEMKEKGFVPNV | | LELFEEMKEKGFVPNVVTYTTLLSGLGKAGRL | |
| | 5 | VTYTTLLSGLGKAGRLEEALELFEEMKEKGFVPNV | | EEALELFEEMKEKGFVPNVVTYTTLLSGLGKA | |
| | 6 | VTYTTLLSGLGKAGRLEEALELFEEMKEKGFVPNV | | GRLEEALELFEEMKEKGFVPNVVTYTTLLSGL | |
| | 7 | VTYTTLLSGLGKAGRLEEALELFEEMKEKGFVPNV | | GKAGRLEEALELFEEMKEKGFVPNVVTYTTLL | |
| | 8 | VTYTTLLSGLGKAGRLEEALELFEEMKEKGFVPNV | | SGLGKAGRLEEALELFEEMKEKGFVPNVVTYT | |
| | C terminal side | VTYTTLLSGLGKAG | 312 | TLLSGLGKAG | 361 |

### Example 6

### Evaluation of Generality of Amino Acid Sequences Capable of Imparting DNA-Binding Ability

All the above examinations were performed by using crPPR (7L/31F). Therefore, it was examined whether a DNA-binding ability can also be imparted to other PPRs by introducing A.A 9A, A.A. 18K, A.A. 31I, A.A. 32K, and A.A. 9A/10Y. In this experiment, it was examined whether DNA-binding abilities of modified naturally occurring type dPPRs, P63 and GUN1, in which A.A. 9A/10Y/18K/31I, and A.A. 31I/32K were introduced into all the motifs thereof were increased. The DNA-binding ability was analyzed in the same manner as that used in Example 3. In this example, the positions of A.A. 31I and A.A. 32K in a motif were determined on the basis of the next motif. Specifically, the position of A.A. 31I was determined so as to be a position locating upstream from No. 1 amino acid of the next PPR motif by 5 amino acids, and the position of A.A.32K was determined so as to be a position locating upstream from No. 1 amino acid of the next PPR motif by 4 amino acids. In the case of the motif at the C-terminus (no next PPR motif), the amino acids of the 5th and 4th positions from the last amino acid (C-terminus side) among those constituting the motif were determined to be A.A. 31I and A.A. 32K, respectively.

### RESULTS AND DISCUSSION

The DNA-binding powers of modified type and naturally occurring type dPPRs were compared by t-test at a significance level of 5% (p<0.06). As a result, DNA-binding powers of P63 and GUN1 introduced with any of the amino acid sequences were increased (FIG. 6). These results revealed that the impartation of DNA-binding ability by introduction of A.A. 9A, A.A. 18K, A.A. 31I, A.A. 32K, and A.A. 9A/10Y is also effective for PPR proteins other than crPPR (7L/31F).

The sequences of the modified type rPPR motifs prepared by this example are shown in the following tables.

### Table 8-1

[symbol]

### Table 8-2

[symbol]

### REFERENCE CITED IN THE SECTION OF EXAMPLES

Non-patent-document 15: Coquille et al., 2014, An artificial PPR scaffold for programmable RNA recognition http://www.nature.com/ncomms/2014/141217/ncomms6729/abs/ncomms6729.html

### SEQUENCE LISTING FREE TEXT

SEQ ID NO: 1, p63 protein
SEQ ID NO: 2, GUN1 protein
SEQ ID NO: 3, pTac2 protein
SEQ ID NO: 4, DG1 protein
SEQ ID NO: 5, GRP23 protein
SEQ ID NO: 6, FokI nuclease domain
SEQ ID NOS: 7 to 214, dPPRs
SEQ ID NOS: 215 to 283, known rPPRs
SEQ ID NO: 284, crPPR
SEQ ID NO: 285, modified type crPPR-1
SEQ ID NO: 286, modified type crPPR-2
SEQ ID NO: 287, modified type crPPR-3
SEQ ID NO: 288, modified type crPPR-4
SEQ ID NO: 289, modified type crPPR-5
SEQ ID NO: 290, modified type crPPR-6
SEQ ID NOS: 291 to 308, At1g10910, At1g26460, At3g15590, At3g59040, At5g10690, At5g24830, At5g67570, At3g42630, At5g42310, At1g12700, At1g30610, At2g35130, At2g41720, At3g18110, At3g53170, At4g21170, At5g48730, At5g50280
SEQ ID NO: 309, crPPR N terminal side
SEQ ID NO: 310, crPPR C terminal side
SEQ ID NOS: 311 to 334, modified type rPPR motifs or C terminal sides
SEQ ID NOS: 335 to 361, modified-type rPPR proteins (full length)
SEQ ID NOS: 362 to 423, N/C terminal sides, or motifs of original/modified type of p63 or GUN1
SEQ ID NOS: 424 to 427, modified-type p63 or GUN1 proteins (full length)

1-14. (canceled)

15. A method for designing a protein that binds to a DNA base or DNA having a specific base sequence, which comprises making the protein contain one or more PPR motifs having a structure of the following formula 1:
[Chemical Formula 2]
(Helix A)-X-(Helix B)-L (Formula 1)
(wherein, in the formula 1:
Helix A is a part that can form an α-helix structure;
X does not exist, or is a part consisting of 1 to 9 amino acids;
Helix B is a part that can form an α-helix structure; and
L is a part consisting of 2 to 7 amino acids),
wherein,
under the following definitions:
the first amino acid of Helix A is referred to as No. 1 amino acid (No. 1 A.A.), the fourth amino acid as No. 4 amino acid (No. 4 A.A.), and
when a next PPR motif (Mₙ₊₁) contiguously exists on the C-terminus side of the PPR motif (Mₙ) (when there is no amino acid insertion between the PPR motifs), the −2nd amino acid counted from the end (C-terminus side) of the amino acids constituting the PPR motif (Mₙ);
when a non-PPR motif consisting of 1 to 20 amino acids exists between the PPR motif (Mₙ) and the next PPR motif (Mₙ₊₁) on the C-terminus side, the amino acid locating upstream of the first amino acid of the next PPR motif (Mₙ₊₁) by 2 positions, i.e., the −2nd amino acid; or
when any next PPR motif (Mₙ₊₁) does not exist on the C-terminus side of the PPR motif (Mₙ), or 21 or more amino acids constituting a non-PPR motif exist between the PPR motif (Mₙ) and the next PPR motif (Mₙ₊₁) on the C-terminus side, the 2nd amino acid counted from the end (C-terminus side) of the amino acids constituting the PPR motif (Mₙ)
is referred to as No. “ii” (−2) amino acid (No. “ii” (−2) A.A.),
one PPR motif (Mₙ) contained in the protein is a PPR motif having a specific combination of amino acids corresponding to a target DNA base or target DNA base sequence as the three amino acids of No. 1 A.A., No. 4 A.A., and No. “ii” (−2) A.A, and satisfies at least one selected from the group consisting of the following conditions (b) to (h):
(b) No. 9 A.A. of the PPR motif (Mₙ) is alanine (A);
(c) No. 10 A.A. of the PPR motif (Mₙ) is tyrosine (Y), phenylalanine (F), or tryptophan (W);
(d) No. 18 A.A. of the PPR motif (Mₙ) is lysine (K), arginine (R), or histidine (H);
(e) No. 20 A.A. of the PPR motif (Mₙ) is glutamic acid (E), or aspartic acid (D);
(f) No. 29 A.A. of the PPR motif (Mₙ) is glutamic acid (E), or aspartic acid (D);
(g) No. 31 A.A. of the PPR motif (Mₙ) is isoleucine (I), leucine (L), or valine (V); and
(h) No. 32 A.A. of the PPR motif (Mₙ) is lysine (K), arginine (R), or histidine (H).

16. The method according to claim 15, wherein the combination of the three amino acids of No. 1 A.A., No. 4 A.A., and No. “ii” (−2) A.A. is determined according to any one of the following definitions:
(1-1) when No. 4 A.A. is glycine (G), No. 1 A.A. may be an arbitrary amino acid, and No. “ii” (−2) A.A. is aspartic acid (D), asparagine (N), or serine (S);
(1-2) when No. 4 A.A. is isoleucine (I), each of No. 1 A.A. and No. “ii” (−2) A.A. may be an arbitrary amino acid;
(1-3) when No. 4 A.A. is leucine (L), each of No. 1 A.A. and No. “ii” (−2) A.A. may be an arbitrary amino acid;
(1-4) when No. 4 A.A. is methionine (M), each of No. 1 A.A. and No. “ii” (−2) A.A. may be an arbitrary amino acid;
(1-5) when No. 4 A.A. is asparagine (N), each of No. 1 A.A. and No. “ii” (−2) A.A. may be an arbitrary amino acid;
(1-6) when No. 4 A.A. is proline (P), each of No. 1 A.A. and No. “ii” (−2) A.A. may be an arbitrary amino acid;
(1-7) when No. 4 A.A. is serine (S), each of No. 1 A.A. and No. “ii” (−2) A.A. may be an arbitrary amino acid;
(1-8) when No. 4 A.A. is threonine (T), each of No. 1 A.A. and No. “ii” (−2) A.A. may be an arbitrary amino acid; and
(1-9) when No. 4 A.A. is valine (V), each of No. 1 A.A. and No. “ii” (−2) A.A. may be an arbitrary amino acid.

17. The method according to claim 15, wherein the combination of the three amino acids of No. 1 A.A., No. 4 A.A., and No. “ii” (−2) A.A. is determined according to any one of the following definitions:
(2-1) when the three amino acids, No. 1 A.A., No. 4 A.A., and No. “ii” (−2) A.A. are an arbitrary amino acid, glycine, and aspartic acid, respectively, the PPR motif selectively binds to G;
(2-2) when the three amino acids, No. 1 A.A., No. 4 A.A., and No. “ii” (−2) A.A., are glutamic acid, glycine, and aspartic acid, respectively, the PPR motif selectively binds to G;
(2-3) when the three amino acids, No. 1 A.A., No. 4 A.A., and No. “ii” (−2) A.A., are an arbitrary amino acid, glycine, and asparagine, respectively, the PPR motif selectively binds to A;
(2-4) when the three amino acids, No. 1 A.A., No. 4 A.A., and No. “ii” (−2) A.A., are glutamic acid, glycine, and asparagine, respectively, the PPR motif selectively binds to A;
(2-5) when the three amino acids, No. 1 A.A., No. 4 A.A., and No. “ii” (−2) A.A., are an arbitrary amino acid, glycine, and serine, respectively, the PPR motif selectively binds to A, and next binds to C;
(2-6) when the three amino acids, No. 1 A.A., No. 4 A.A., and No. “ii” (−2) A.A., are an arbitrary amino acid, isoleucine, and an arbitrary amino acid, respectively, the PPR motif selectively binds to T and C;
(2-7) when the three amino acids, No. 1 A.A., No. 4 A.A., and No. “ii” (−2) A.A., are an arbitrary amino acid, isoleucine, and asparagine, respectively, the PPR motif selectively binds to T, and next binds to C;
(2-8) when the three amino acids, No. 1 A.A., No. 4 A.A., and No. “ii” (−2) A.A., are an arbitrary amino acid, leucine, and an arbitrary amino acid, respectively, the PPR motif selectively binds to T and C;
(2-9) when the three amino acids, No. 1 A.A., No. 4 A.A., and No. “ii” (−2) A.A., are an arbitrary amino acid, leucine, and aspartic acid, respectively, the PPR motif selectively binds to C;
(2-10) when the three amino acids, No. 1 A.A., No. 4 A.A., and No. “ii” (−2) A.A., are an arbitrary amino acid, leucine, and lysine, respectively, the PPR motif selectively binds to T;
(2-11) when the three amino acids, No. 1 A.A., No. 4 A.A., and No. “ii” (−2) A.A., are an arbitrary amino acid, methionine, and an arbitrary amino acid, respectively, the PPR motif selectively binds to T;
(2-12) when the three amino acids, No. 1 A.A., No. 4 A.A., and No. “ii” (−2) A.A., are an arbitrary amino acid, methionine, and aspartic acid, respectively, the PPR motif selectively binds to T;
(2-13) when the three amino acids, No. 1 A.A., No. 4 A.A., and No. “ii” (−2) A.A., are isoleucine, methionine, and aspartic acid, respectively, the PPR motif selectively binds to T, and next binds to C;
(2-14) when the three amino acids, No. 1 A.A., No. 4 A.A., and No. “ii” (−2) A.A., are an arbitrary amino acid, asparagine, and an arbitrary amino acid, respectively, the PPR motif selectively binds to C and T;
(2-15) when the three amino acids, No. 1 A.A., No. 4 A.A., and No. “ii” (−2) A.A., are an arbitrary amino acid, asparagine, and aspartic acid, respectively, the PPR motif selectively binds to T;
(2-16) when the three amino acids, No. 1 A.A., No. 4 A.A., and No. “ii” (−2) A.A., are phenylalanine, asparagine, and aspartic acid, respectively, the PPR motif selectively binds to T;
(2-17) when the three amino acids, No. 1 A.A., No. 4 A.A., and No. “ii” (−2) A.A., are glycine, asparagine, and aspartic acid, respectively, the PPR motif selectively binds to T;
(2-18) when the three amino acids, No. 1 A.A., No. 4 A.A., and No. “ii” (−2) A.A., are isoleucine, asparagine, and aspartic acid, respectively, the PPR motif selectively binds to T;
(2-19) when the three amino acids, No. 1 A.A., No. 4 A.A., and No. “ii” (−2) A.A., are threonine, asparagine, and aspartic acid, respectively, the PPR motif selectively binds to T;
(2-20) when the three amino acids, No. 1 A.A., No. 4 A.A., and No. “ii” (−2) A.A. are valine, asparagine, and aspartic acid, respectively, the PPR motif selectively binds to T, and next binds to C;
(2-21) when the three amino acids, No. 1 A.A., No. 4 A.A., and No. “ii” (−2) A.A. are tyrosine, asparagine, and aspartic acid, respectively, the PPR motif selectively binds to T, and next binds to C;
(2-22) when the three amino acids, No. 1 A.A., No. 4 A.A., and No. “ii” (−2) A.A., are an arbitrary amino acid, asparagine, and asparagine, respectively, the PPR motif selectively binds to C;
(2-23) when the three amino acids, No. 1 A.A., No. 4 A.A., and No. “ii” (−2) A.A., are isoleucine, asparagine, and asparagine, respectively, the PPR motif selectively binds to C;
(2-24) when the three amino acids, No. 1 A.A., No. 4 A.A., and No. “ii” (−2) A.A., are serine, asparagine, and asparagine, respectively, the PPR motif selectively binds to C;
(2-25) when the three amino acids, No. 1 A.A., No. 4 A.A., and No. “ii” (−2) A.A., are valine, asparagine, and asparagine, respectively, the PPR motif selectively binds to C;
(2-26) when the three amino acids, No. 1 A.A., No. 4 A.A., and No. “ii” (−2) A.A., are an arbitrary amino acid, asparagine, and serine, respectively, the PPR motif selectively binds to C;
(2-27) when the three amino acids, No. 1 A.A., No. 4 A.A., and No. “ii” (−2) A.A., are valine, asparagine, and serine, respectively, the PPR motif selectively binds to C;
(2-28) when the three amino acids, No. 1 A.A., No. 4 A.A., and No. “ii” (−2) A.A., are an arbitrary amino acid, asparagine, and threonine, respectively, the PPR motif selectively binds to C;
(2-29) when the three amino acids, No. 1 A.A., No. 4 A.A., and No. “ii” (−2) A.A., are valine, asparagine, and threonine, respectively, the PPR motif selectively binds to C;
(2-30) when the three amino acids, No. 1 A.A., No. 4 A.A., and No. “ii” (−2) A.A., are an arbitrary amino acid, asparagine, and tryptophan, respectively, the PPR motif selectively binds to C, and next binds to T;
(2-31) when the three amino acids, No. 1 A.A., No. 4 A.A., and No. “ii” (−2) A.A., are isoleucine, asparagine, and tryptophan, respectively, the PPR motif selectively binds to T, and next binds to C;
(2-32) when the three amino acids, No. 1 A.A., No. 4 A.A., and No. “ii” (−2) A.A., are an arbitrary amino acid, proline, and an arbitrary amino acid, respectively, the PPR motif selectively binds to T;
(2-33) when the three amino acids, No. 1 A.A., No. 4 A.A., and No. “ii” (−2) A.A., are an arbitrary amino acid, proline, and aspartic acid, respectively, the PPR motif selectively binds to T;
(2-34) when the three amino acids, No. 1 A.A., No. 4 A.A., and No. “ii” (−2) A.A., are phenylalanine, proline, and aspartic acid, respectively, the PPR motif selectively binds to T;
(2-35) when the three amino acids, No. 1 A.A., No. 4 A.A., and No. “ii” (−2) A.A., are tyrosine, proline, and aspartic acid, respectively, the PPR motif selectively binds to T;
(2-36) when the three amino acids, No. 1 A.A., No. 4 A.A., and No. “ii” (−2) A.A., are an arbitrary amino acid, serine, and an arbitrary amino acid, respectively, the PPR motif selectively binds to A and G;
(2-37) when the three amino acids, No. 1 A.A., No. 4 A.A., and No. “ii” (−2) A.A., are an arbitrary amino acid, serine, and asparagine, respectively, the PPR motif selectively binds to A;
(2-38) when the three amino acids, No. 1 A.A., No. 4 A.A., and No. “ii” (−2) A.A., are phenylalanine, serine, and asparagine, respectively, the PPR motif selectively binds to A;
(2-39) when the three amino acids, No. 1 A.A., No. 4 A.A., and No. “ii” (−2) A.A., are valine, serine, and asparagine, respectively, the PPR motif selectively binds to A;
(2-40) when the three amino acids, No. 1 A.A., No. 4 A.A., and No. “ii” (−2) A.A., are an arbitrary amino acid, threonine, and an arbitrary amino acid, respectively, the PPR motif selectively binds to A and G;
(2-41) when the three amino acids, No. 1 A.A., No. 4 A.A., and No. “ii” (−2) A.A., are an arbitrary amino acid, threonine, and aspartic acid, respectively, the PPR motif selectively binds to G;
(2-42) when the three amino acids, No. 1 A.A., No. 4 A.A., and No. “ii” (−2) A.A., are valine, threonine, and aspartic acid, respectively, the PPR motif selectively binds to G;
(2-43) when the three amino acids, No. 1 A.A., No. 4 A.A., and No. “ii” (−2) A.A., are an arbitrary amino acid, threonine, and asparagine, respectively, the PPR motif selectively binds to A;
(2-44) when the three amino acids, No. 1 A.A., No. 4 A.A., and No. “ii” (−2) A.A., are phenylalanine, threonine, and asparagine, respectively, the PPR motif selectively binds to A;
(2-45) when the three amino acids, No. 1 A.A., No. 4 A.A., and No. “ii” (−2) A.A., are isoleucine, threonine, and asparagine, respectively, the PPR motif selectively binds to A;
(2-46) when the three amino acids, No. 1 A.A., No. 4 A.A., and No. “ii” (−2) A.A., are valine, threonine, and asparagine, respectively, the PPR motif selectively binds to A;
(2-47) when the three amino acids, No. 1 A.A., No. 4 A.A., and No. “ii” (−2) A.A., are an arbitrary amino acid, valine, and an arbitrary amino acid, respectively, the PPR motif binds with A, C, and T, but does not bind to G;
(2-48) when the three amino acids, No. 1 A.A., No. 4 A.A., and No. “ii” (−2) A.A., are isoleucine, valine, and aspartic acid, respectively, the PPR motif selectively binds to C, and next binds to A;
(2-49) when the three amino acids, No. 1 A.A., No. 4 A.A., and No. “ii” (−2) A.A., are an arbitrary amino acid, valine, and glycine, respectively, the PPR motif selectively binds to C; and
(2-50) when the three amino acids, No. 1 A.A., No. 4 A.A., and No. “ii” (−2) A.A., are an arbitrary amino acid, valine, and threonine, respectively, the PPR motif selectively binds to T.

18. The method according to claim 15, wherein at least one selected from the group consisting of the combination of (b) and (c), the combination of (d) and (e), (g), and (h) is satisfied.

19. The method according to claim 18, wherein the combination of (b) and (c) is satisfied, and at least one selected from the group consisting of the combination of (d) and (e), (g), and (h) is satisfied.

20. The method according to claim 19, wherein the combination of (b) and (c), the combination of (d) and (e), and (g) are satisfied.

21. The method according to claim 15, wherein the protein contains a plurality of PPR motifs, and has a DNA-binding PPR motif content of 13% or higher.

22. A method for producing a protein, which comprises designing a protein by the method according to claim 15, and producing the designed protein.

23. (canceled)

24. A method for editing a genome, which comprises
designing a protein by the method according to claim 15, binding a region consisting of the designed protein and a functional region to produce a complex, and using the produced complex provided that implementation in a human individual is excluded.

25. (canceled)
